(19) United States
(12) Patent Application Publication
(10) Pub. No.: US 2018/0214006 A1
(43) Pub. Date: Aug. 2, 2018

(54) IMAGE PROCESSING APPARATUS AND IMAGE PROCESSING METHOD

(71) Applicant: OLYMPUS CORPORATION, Tokyo (JP)

(72) Inventors: Syunya AKIMOTO, Kawasaki-shi (JP); Seiichi ITO, Tokyo (JP); Junichi ONISHI, Tokyo (JP)

(73) Assignee: OLYMPUS CORPORATION, Tokyo (JP)

(21) Appl. No.: 15/938,461

(22) Filed: Mar. 28, 2018

Related U.S. Application Data
(63) Continuation of application No. PCT/JP2016/078396, filed on Sep. 27, 2016.

(30) Foreign Application Priority Data
Sep. 28, 2015 (JP) 2015-190133

(51) Int. Cl. A61B 1/00; A61B 1/04; A61B 1/06; A61B 1/07; A61B 1/307; A61B 5/06

(52) U.S. Cl. CPC A61B 1/00009; A61B 1/04; A61B 1/00022; A61B 1/00117; A61B 1/00126; A61B 2090/367; A61B 1/07; A61B 1/307; A61B 5/062; A61B 1/00045; A61B 1/0669

(57) ABSTRACT

An image processing apparatus includes: a three-dimensional model structuring section configured to generate, when an image pickup signal related to a region in a subject is inputted from an image pickup apparatus configured to pick up an image of an inside of the subject, three-dimensional data representing a shape of the region based on the image pickup signal; and an image generation section configured to perform, on the three-dimensional data generated by the three-dimensional model structuring section, processing of allowing visual recognition of a boundary region between a structured region that is a region, an image of which is picked up by the image pickup apparatus, and an unstructured region that is a region, an image of which is yet to be picked up by the image pickup apparatus, and generate a three-dimensional image.

### CROSS REFERENCE TO RELATED APPLICATION

This application is a continuation application of PCT/JP2016/078396 filed on Sep. 27, 2016 and claims benefit of Japanese Application No. 2015-190133 filed in Japan on Sep. 28, 2015, the entire contents of which are incorporated herein by this reference.

### BACKGROUND OF INVENTION

### 1. Field of the Invention

The present invention relates to an image processing apparatus and an image processing method that observe a subject by using an endoscope.

### 2. Description of the Related Art

Recently, an endoscope system using an endoscope has been widely used in medical and industrial fields. For example, in the medical field, an endoscope needs to be inserted into an organ having a complicated luminal shape in a subject to observe or examine an inside of the organ in detail in some cases.

For example, a conventional example of Japanese Patent No. 5354494 proposes an endoscope system that generates and displays a luminal shape of the organ from an endoscope image picked up by an endoscope to present a region observed by the endoscope.

### SUMMARY OF THE INVENTION

An image processing apparatus according to an aspect of the present invention includes: a three-dimensional model structuring section configured to generate, when an image pickup signal related to a region in a subject is inputted from an image pickup apparatus configured to pick up an image of an inside of the subject, three-dimensional data representing a shape of the region based on the image pickup signal; and an image generation section configured to perform, on the three-dimensional data generated by the three-dimensional model structuring section, processing of allowing visual recognition of a boundary region between a structured region that is a region, an image of which is picked up by the image pickup apparatus, and an unstructured region that is a region, an image of which is yet to be picked up by the image pickup apparatus, and generate a three-dimensional image.

An image processing method according to an aspect of the present invention includes: generating, by a three-dimensional model structuring section, when an image pickup signal related to a region in a subject is inputted from an image pickup apparatus configured to pick up an image of an inside of the subject, three-dimensional data representing a shape of the region based on the image pickup signal; and performing, by an image generation section, on the three-dimensional data generated by the three-dimensional model structuring section, processing of allowing visual recognition of a boundary region between a structured region that is a region, an image of which is picked up by the image pickup apparatus, and an unstructured region that is a region, an image of which is yet to be picked up by the image pickup apparatus, and generating a three-dimensional image.

### BRIEF DESCRIPTION OF THE DRAWINGS

FIG. 1 is a diagram illustrating the entire configuration of an endoscope system according to a first embodiment of the present invention;

FIG. 2 is a diagram illustrating the configuration of an image processing apparatus in the first embodiment;

FIG. 3A is an explanatory diagram illustrating renal pelvis and calyx in a state in which an insertion section of an endoscope is inserted;

FIG. 3B is a diagram illustrating an exemplary situation in which a 3D model image displayed on a monitor in accordance with change of an observation region along with an insertion operation of the endoscope is updated;

FIG. 3C is a diagram illustrating an exemplary situation in which the 3D model image displayed on the monitor in accordance with change of the observation region along with the insertion operation of the endoscope is updated;

FIG. 3D is a diagram illustrating an exemplary situation in which the 3D model image displayed on the monitor in accordance with change of the observation region along with the insertion operation of the endoscope is updated;

FIG. 4 is a diagram illustrating a relation between a front surface corresponding to the order of apexes of a triangle as a polygon used to structure a 3D model image, and a normal vector;

FIG. 5 is a flowchart illustrating processing of an image processing method according to the first embodiment;

FIG. 6 is a flowchart illustrating contents of processing according to the first embodiment;

FIG. 7 is an explanatory diagram illustrating a situation in which polygons are set on a 3D-shaped surface;

FIG. 8 is a flowchart illustrating detail of processing of setting the normal vector in FIG. 6 and determining an inner surface and an outer surface;

FIG. 9 is a diagram illustrating a polygon list produced when polygons are set as illustrated in FIG. 7;

FIG. 10 is a diagram illustrating a polygon list generated by setting a normal vector for the polygon list illustrated in FIG. 9;

FIG. 11 is a diagram illustrating a situation in which normal vectors are set to respective adjacent polygons set to draw an observed inner surface;

FIG. 12 is an explanatory diagram of an operation of determining the direction of a normal vector by using position information of a position sensor when the position sensor is provided at a distal end portion;

FIG. 13 is a diagram illustrating a 3D model image displayed on the monitor when enhanced display is not selected;

FIG. 14 is a diagram schematically illustrating the periphery of a boundary in a 3D model image;

FIG. 15 is a diagram illustrating a polygon list corresponding to the case illustrated in FIG. 14;

FIG. 16 is a diagram illustrating a boundary list produced by extraction of boundary sides;

FIG. 17 is a diagram illustrating a 3D model image displayed on the monitor when enhanced display is selected;

FIG. 18 is a flowchart illustrating contents of processing in a first modification of the endoscope system according to the first embodiment;

FIG. 19 is an explanatory diagram for description of the operation illustrated in FIG. 18;

FIG. 20 is a diagram illustrating a 3D model image displayed on the monitor when enhanced display is selected in the first modification;

FIG. 21 is a flowchart illustrating contents of processing in a second modification of the endoscope system according to the first embodiment;

FIG. 22 is an explanatory diagram of processing in the second modification;

FIG. 23 is a diagram illustrating a 3D model image generated by the second modification and displayed on the monitor;

FIG. 24 is a flowchart illustrating contents of processing in a third modification of the endoscope system according to the first embodiment;

FIG. 25 is an explanatory diagram of processing in the third modification;

FIG. 26 is a diagram illustrating a 3D model image generated by the third modification and displayed on the monitor;

FIG. 27 is a flowchart illustrating contents of processing in a fourth modification of the endoscope system according to the first embodiment;

FIG. 28 is an explanatory diagram of processing in the fourth modification;

FIG. 29 is a diagram illustrating a 3D model image generated by the fourth modification and displayed on the monitor;

FIG. 30A is a diagram illustrating the configuration of an image processing apparatus in a fifth modification of the first embodiment;

FIG. 30B is a flowchart illustrating contents of processing in the fifth modification of the endoscope system according to the first embodiment;

FIG. 31 is a diagram illustrating a 3D model image generated by the fifth modification and displayed on the monitor;

FIG. 32 is a flowchart illustrating contents of processing in a sixth modification of the endoscope system according to the first embodiment;

FIG. 33 is a diagram illustrating a 3D model image generated by the sixth modification and displayed on the monitor;

FIG. 34 is a diagram illustrating the configuration of an image processing apparatus in a seventh modification of the first embodiment;

FIG. 35 is a flowchart illustrating contents of processing in the seventh modification;

FIG. 36 is a diagram illustrating a 3D model image generated by the seventh modification and displayed on the monitor when enhanced display and index display are selected;

FIG. 37 is a diagram illustrating a 3D model image generated by the seventh modification and displayed on the monitor when enhanced display is not selected but index display is selected;

FIG. 38 is a flowchart illustrating contents of processing of generating an index in an eighth modification of the first embodiment;

FIG. 39 is an explanatory diagram of FIG. 38;

FIG. 40 is an explanatory diagram of a modification of FIG. 38;

FIG. 41 is a diagram illustrating a 3D model image generated by the eighth modification and displayed on the monitor;

FIG. 42 is a diagram illustrating the configuration of an image processing apparatus in a ninth modification of the first embodiment;

FIG. 43A is a diagram illustrating a 3D model image generated by the ninth modification and displayed on the monitor;

FIG. 43B is a diagram illustrating a 3D model image before being rotated;

FIG. 43C is a diagram illustrating a 3D model image before being rotated;

FIG. 43D is an explanatory diagram when an unstructured region is displayed in an enlarged manner;

FIG. 44 is a diagram illustrating the configuration of an image processing apparatus in a tenth modification of the first embodiment;

FIG. 45 is a diagram illustrating 3D shape data including a boundary across which a threshold is exceeded or not;

FIG. 46 is a diagram illustrating 3D shape data of a target of determination by a determination section and the direction of an axis of a primary component of the 3D shape data;

FIG. 47 is a diagram obtained by projecting the coordinates of a boundary illustrated in FIG. 46 onto a plane orthogonal to an axis of a first primary component;

FIG. 48 is a diagram illustrating the configuration of an image processing apparatus in an eleventh modification of the first embodiment;

FIG. 49 is a flowchart illustrating contents of processing in the eleventh modification;

FIG. 50 is an explanatory diagram of processing in the eleventh modification;

FIG. 51 is a diagram illustrating a core line image generated by the eleventh modification; and

FIG. 52 is a diagram illustrating the configuration of an image processing apparatus in a twelfth modification of the first embodiment.

### DETAILED DESCRIPTION OF THE PREFERRED EMBODIMENTS

Embodiments of the present invention will be described below with reference to the accompanying drawings.

### First Embodiment

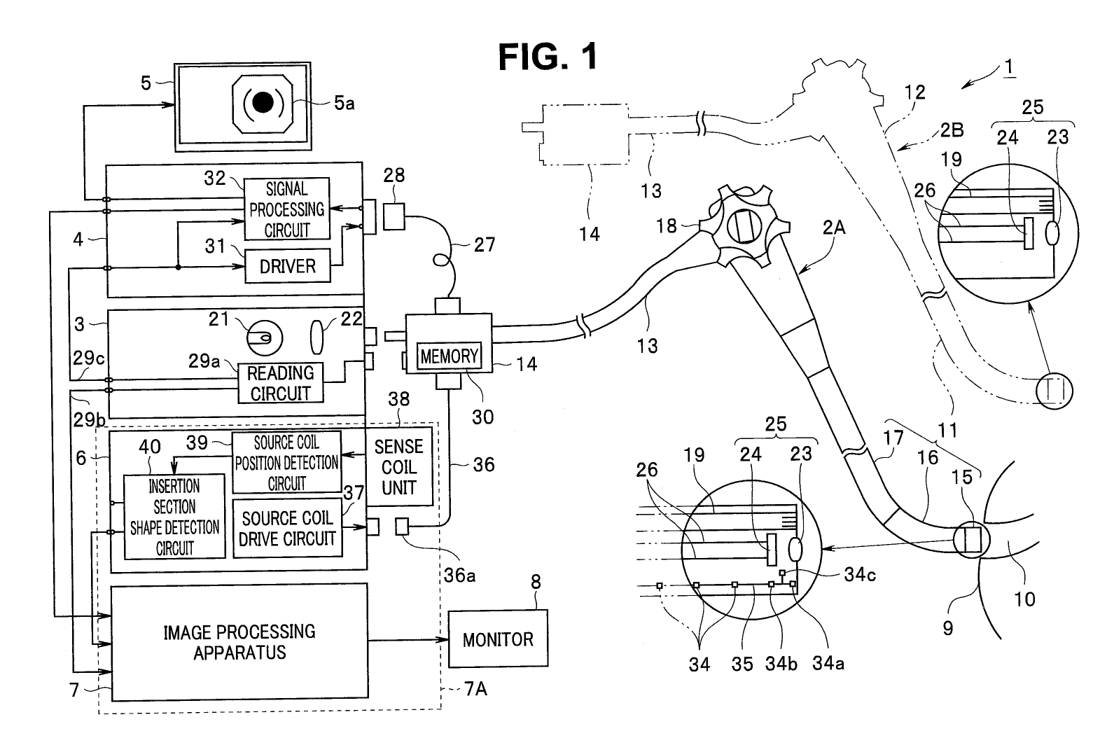

An endoscope system 1 illustrated in FIG. 1 includes an endoscope 2A that is inserted into a subject, a light source apparatus 3 configured to supply illumination light to the endoscope 2A, a video processor 4 as a signal processing apparatus configured to perform signal processing for an image pickup section provided to the endoscope 2A, a monitor 5 as an endoscope image display apparatus configured to display an endoscope image generated by the video processor 4, a UPD apparatus 6 as an insertion section shape detection apparatus configured to detect an insertion section shape of the endoscope 2A through a sensor provided in the endoscope 2A, an image processing apparatus 7 configured to perform image processing of generating a three-dimensional (also abbreviated as 3D) model image from a two-dimensional image, and a monitor 8 as a display apparatus configured to display the 3D model image generated by the image processing apparatus 7. Note that an image processing apparatus 7A including the UPD apparatus 6 as illustrated with a dotted line may be used in place of the image processing apparatus 7 separately provided from the UPD apparatus 6 illustrated with a solid line in FIG. 1. The UPD apparatus 6 may be omitted when position information is estimated from an image in the processing of generating a three-dimensional model image.

The endoscope 2A includes an insertion section 11 that is inserted into, for example, a ureter 10 as part of a predetermined luminal organ (also simply referred to as a luminal organ) that is a subject to be observed in a patient 9, an operation section 12 provided at a rear end (base end) of the insertion section 11, and an universal cable 13 extending from the operation section 12, and a light guide connector 14 provided at an end part of the universal cable 13 is detachably connected with a light guide connector reception of the light source apparatus 3.

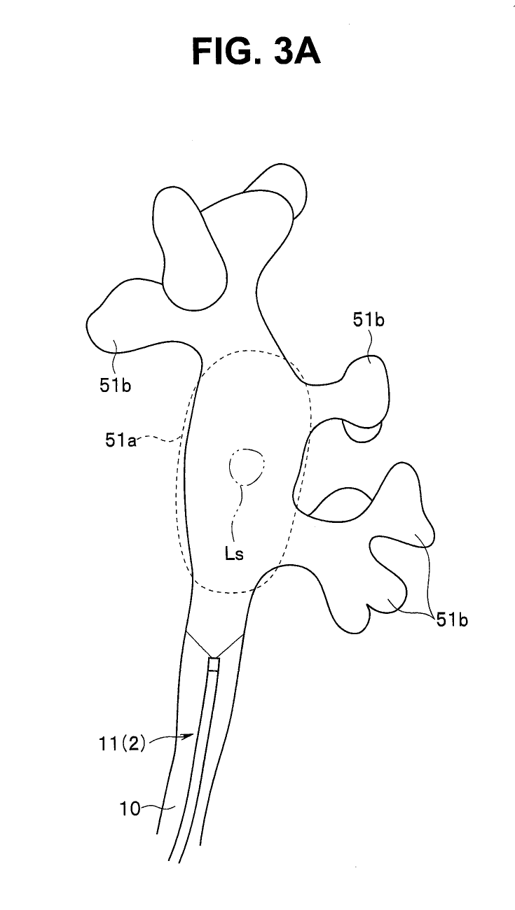

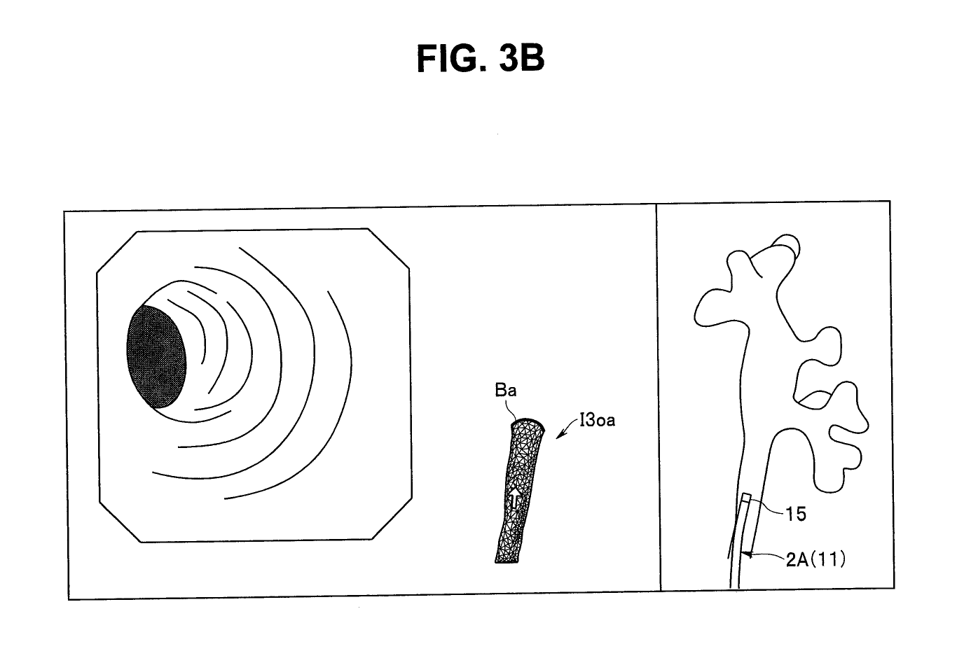

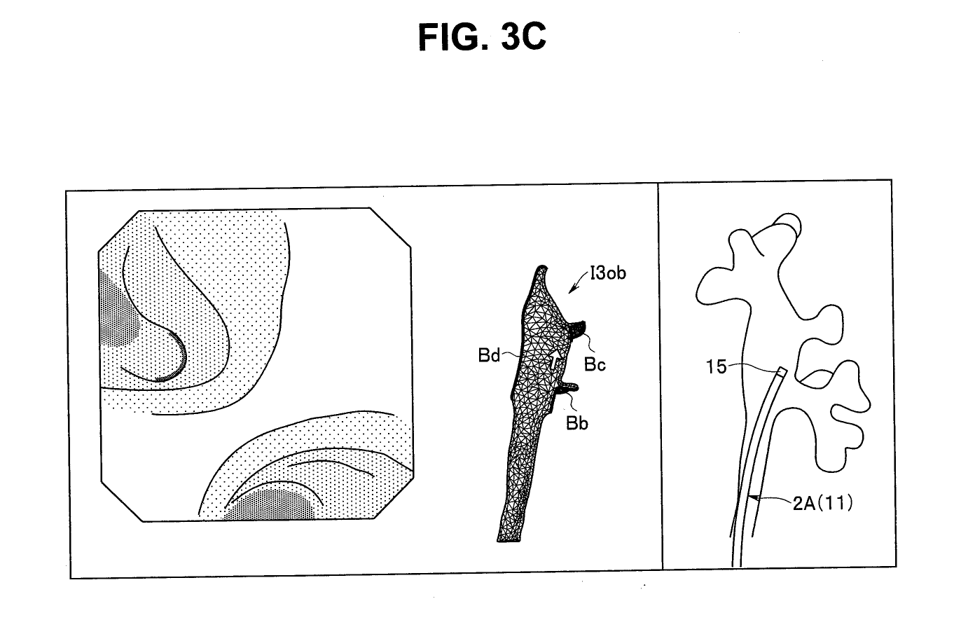

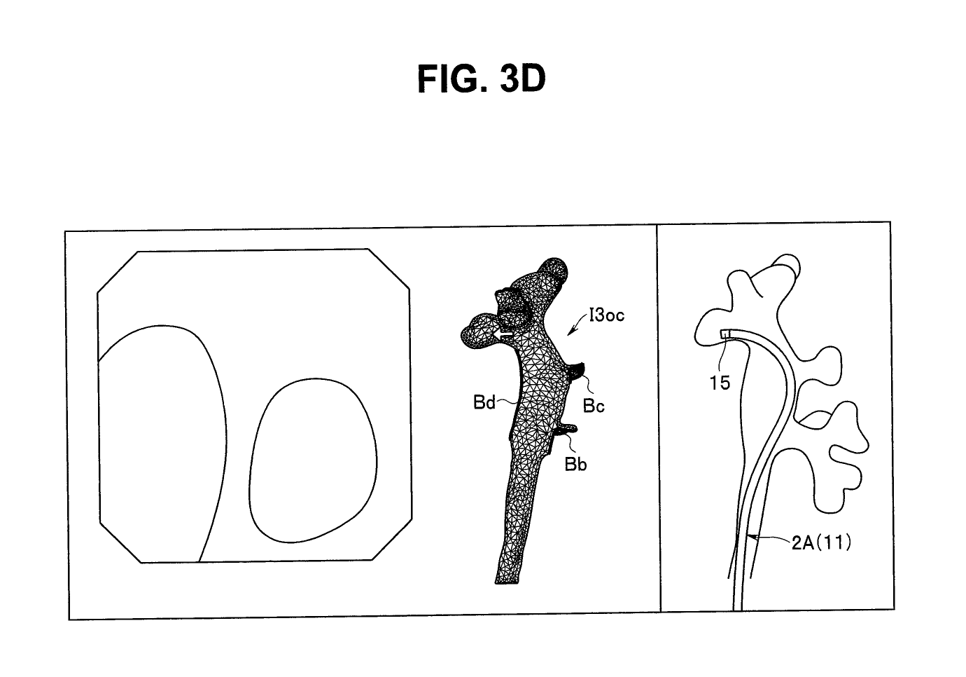

Note that the ureter 10 communicates with a renal pelvis 51a and a renal calyx 51b on a deep part side (refer to FIG. 3A).

The insertion section 11 includes a distal end portion 15 provided at a leading end, a bendable bending portion 16 provided at a rear end of the distal end portion 15, and a flexible pipe section 17 extending from a rear end of the bending portion 16 to a front end of the operation section 12.

The operation section 12 is provided with a bending operation knob 18 for a bending operation of the bending portion 16.

As illustrated in a partially enlarged view in FIG. 1, a light guide 19 that transmits illumination light is inserted in the insertion section 11, and a leading end of the light guide 19 is attached to an illumination window of the distal end portion 15, whereas a rear end of the light guide 19 reaches the light guide connector 14.

Illumination light generated at a light source lamp 21 of the light source apparatus 3 is condensed through a light condensing lens 22 and incident on the light guide connector 14, and the light guide 19 emits transmitted illumination light from a leading surface attached to the illumination window.

An optical image of an observation target site (also referred to as an object) in the luminal organ illuminated with an illumination light is formed at an imaging position of an objective optical system 23 through the objective optical system 23 attached to an observation window (image pickup window) provided adjacent to the illumination window of the distal end portion 15. The image pickup plane of, for example, a charge-coupled device (abbreviated as CCD) 24 as an image pickup device is disposed at the imaging position of the objective optical system 23. The CCD 24 has a predetermined view angle.

The objective optical system 23 and the CCD 24 serve as an image pickup section (or image pickup apparatus) 25 configured to pick up an image of the inside of the luminal organ. Note that the view angle of the CCD 24 also depends on an optical property (for example, the focal length) of the objective optical system 23, and thus may be referred to as the view angle of the image pickup section 25 with taken into consideration the optical property of the objective optical system 23 or the view angle of observation using the objective optical system.

The CCD 24 is connected with one end of a signal line 26 inserted in, for example, the insertion section 11, and the other end of the signal line 26 extends to a signal connector 28 at an end part of the connection cable 27 through a connection cable 27 (or a signal line inside the connection cable 27) connected with the light guide connector 14. The signal connector 28 is detachably connected with a signal connector reception of the video processor 4.

The video processor 4 includes a driver 31 configured to generate a CCD drive signal, and a signal processing circuit 32 configured to perform signal processing on an output signal from the CCD 24 to generate an image signal (video signal) to be displayed as an endoscope image on the monitor 5. The driver 31 applies the CCD drive signal to the CCD 24 through, for example, the signal line 26, and upon the application of the CCD drive signal, the CCD 24 outputs, as an output signal, an image pickup signal obtained through optical-electrical conversion of an optical image formed on the image pickup plane.

Namely, the image pickup section 25 includes the objective optical system 23 and the CCD 24 and is configured sequentially generate a two-dimensional image pickup signal by receiving return light from a region in a subject irradiated with illumination light from the insertion section 11 and output the generated two-dimensional image pickup signal.

The image pickup signal outputted from the CCD 24 is converted into an image signal by the signal processing circuit 32 and the signal processing circuit 32 outputs the image signal to the monitor 5 from an output end of the signal processing circuit 32. The monitor 5 displays an image corresponding to an optical image formed on the image pickup plane of the CCD 24 and picked up at a predetermined view angle (in a range of view angle), as an endoscope image in an endoscope image display area (simply abbreviated as an image display area) 5a. FIG. 1 illustrates a situation in which, when the image pickup plane of the CCD 24 is, for example, a square, an endoscope image substantially shaped in an octagon obtained by truncating the four corners of the square is displayed.

The endoscope 2A includes, for example, in the light guide connector 14, a memory 30 storing information unique to the endoscope 2A, and the memory 30 stores view angle data (or view angle information) as information indicating the view angle of the CCD 24 mounted on the endoscope 2A. When the light guide connector 14 is connected with the light source apparatus 3, a reading circuit 29a provided inside the light source apparatus 3 reads view angle data through an electrical contact connected with the memory 30.

The reading circuit 29a outputs the read view angle data to the image processing apparatus 7 through a communication line 29b. The reading circuit 29a also outputs read data on the number of pixels of the CCD 24 to the driver 31 and the signal processing circuit 32 of the video processor 4 through a communication line 29c. The driver 31 generates a CCD drive signal in accordance with the inputted data on the number of pixels, and the signal processing circuit 32 performs signal processing corresponding to the data on the number of pixels.

Note that the exemplary configuration in FIG. 1 illustrates the case in which the reading circuit 29a configured to read unique information in the memory 30 is provided to the light source apparatus 3, but the reading circuit 29a may be provided to the video processor 4.

The signal processing circuit 32 serves as an input section configured to input generated two-dimensional endoscope image data (also referred to as image data) as, for example, a digital image signal to the image processing apparatus 7.

In the insertion section 11, a plurality of source coils 34 functioning as a sensor configured to detect the insertion shape of the insertion section 11 being inserted into a subject are disposed at an appropriate interval in a longitudinal direction of the insertion section 11. In the distal end portion 15, two source coils 34a and 34b are disposed in the longitudinal direction of the insertion section 11, and a source coil 34c is disposed in, for example, a direction orthogonal to a line segment connecting the two source coils 34a and 34b. The direction of the line segment connecting the source coils 34a and 34b is substantially aligned with an optical axis direction (or sight line direction) of the objective optical system 23 included in the image pickup section 25, and a plane including the three source coils 34a, 34b, and 34c is substantially aligned with an up-down direction of on the image pickup plane of the CCD 24.

Thus, a source coil position detection circuit 39 to be described later inside the UPD apparatus 6 can detect the three-dimensional position of the distal end portion 15 and a longitudinal direction of the distal end portion 15 by detecting the three-dimensional positions of the three source coils 34a, 34b, and 34c, and in other words, the three-dimensional position of the objective optical system 23 included in the image pickup section 25 and disposed at a known distance from each of the three source coils 34a, 34b, and 34c and the sight line direction (optical axis direction) of the objective optical system 23 can be detected by detecting the three-dimensional positions of the three source coils 34a, 34b, and 34c at the distal end portion 15.

The source coil position detection circuit 39 serves as an information acquisition section configured to acquire information on the three-dimensional position and the sight line direction of the objective optical system 23.

Note that the image pickup section 25 in the endoscope 2A illustrated in FIG. 1 has a configuration in which the image pickup plane of the CCD 24 is disposed at the imaging position of the objective optical system 23, but the present invention is applicable to an endoscope including an image pickup section having a configuration in which an image guide that transmits an optical image by the objective optical system 23 is provided between the objective optical system 23 and the CCD.

The plurality of source coils 34 including the three source coils 34a, 34b, and 34c are each connected with one end of the corresponding one of a plurality of signal lines 35, and the other ends of the plurality of signal lines 35 are each connected with a cable 36 extending from the light guide connector 14, and a signal connector 36a at an end part of the cable 36 is detachably connected with a signal connector reception of the UPD apparatus 6.

The UPD apparatus 6 includes a source coil drive circuit 37 configured to drive the plurality of source coils 34 to generate an alternating-current magnetic field around each source coil 34, a sense coil unit 38 including a plurality of sense coils and configured to detect the three-dimensional position of each source coil by detecting a magnetic field generated by the respective source coils, the source coil position detection circuit 39 configured to detect the three-dimensional positions of the respective source coils based on detection signals by the plurality of sense coils, and an insertion section shape detection circuit 40 configured to detect the insertion shape of the insertion section 11 based on the three-dimensional positions of the respective source coils detected by the source coil position detection circuit 39 and generate an image in the insertion shape.

The three-dimensional position of each source coil is detected and managed in a coordinate system of the UPD apparatus 6.

As described above, the source coil position detection circuit 39 serves as an information acquisition section configured to acquire information on the observation position (three-dimensional position) and the sight line direction of the objective optical system 23. In a more limited sense, the source coil position detection circuit 39 and the three source coils 34a, 34b, and 34c serve as an information acquisition section configured to acquire information on the observation position and the sight line direction of the objective optical system 23.

The endoscope system 1 (and the image processing apparatus 7) according to the present embodiment may employ an endoscope 2B illustrated with a double-dotted and dashed line in FIG. 1 (in place of the endoscope 2A).

The endoscope 2B is provided with the insertion section 11 including no source coils 34 in the endoscope 2A. In this endoscope, no source coils 34a, 34b, and 34c are disposed in the distal end portion 15 as illustrated in an enlarged view. When the endoscope 2B is connected with the light source apparatus 3 and the video processor 4, the reading circuit 29a reads unique information in the memory 30 in the light guide connector 14 and outputs the unique information to the image processing apparatus 7. The image processing apparatus 7 recognizes that the endoscope 2B is an endoscope including no source coils.

The image processing apparatus 7 estimates the observation position and the sight line direction of the objective optical system 23 by image processing without using the UPD apparatus 6.

In the endoscope system 1 according to the present embodiment, although not illustrated, the inside of renal pelvis and calyx may be examined by using an endoscope (denoted by 2C) in which the source coils 34a, 34b, and 34c that allow detection of the observation position and the sight line direction of the objective optical system 23 provided to the distal end portion 15 are provided in the distal end portion 15.

In this manner, in the present embodiment, identification information provided to the endoscope 2I (I=A, B, or C) is used to examine the inside of renal pelvis and calyx with any of the endoscope 2A (or 2C) including a position sensor and the endoscope 2B including no position sensor and structure a 3D model image from two-dimensional image data acquired through the examination as described later.

When the endoscope 2A is used, the insertion section shape detection circuit 40 includes a first output end from which an image signal of the insertion shape of the endoscope 2A is outputted, and a second output end from which data (also referred to as position and direction data) on the observation position and the sight line direction of the objective optical system 23 detected by the source coil position detection circuit 39 is outputted. Then, the data on the observation position and the sight line direction is outputted from the second output end to the image processing apparatus 7. Note that the data on the observation position and the sight line direction outputted from the second output end may be outputted from the source coil position detection circuit 39 serving as an information acquisition section.

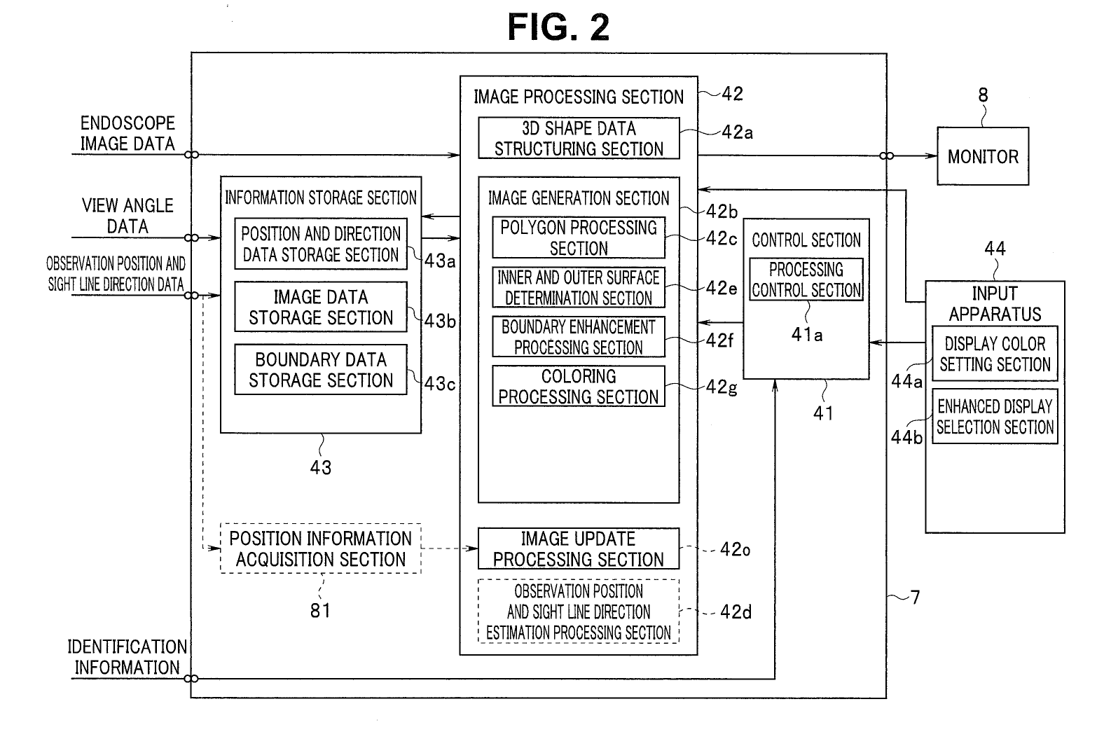

FIG. 2 illustrates the configuration of the image processing apparatus 7. The image processing apparatus 7 includes a control section 41 configured to perform operation control of the image processing apparatus 7, an image processing section 42 configured to generate (or structure) 3D shape data (or 3D model data) and a 3D model image, and an information storage section 43 configured to store information such as image data.

An image signal of the 3D model image generated by the image processing section 42 is outputted to the monitor 8, and the monitor 8 displays the 3D model image generated by the image processing section 42.

The control section 41 and the image processing section 42 are connected with an input apparatus 44 including, for example, a keyboard and a mouse to allow a user such as an operator to perform, through a display color setting section 44a of the input apparatus 44, selection (or setting) of a display color in which a 3D model image is displayed, and to perform, through an enhanced display selection section 44b, selection of enhanced display of a boundary between a structured region and an unstructured region in the 3D model image to facilitate visual recognition. Note that, for example, any parameter for image processing can be inputted to the image processing section 42 through the input apparatus 44.

The control section 41 is configured by, for example, a central processing unit (CPU) and functions as a processing control section 41a configured to control an image processing operation of the image processing section 42 in accordance with setting or selection from the input apparatus 44.

Identification information unique to the endoscope 2I is inputted from the memory 30 to the control section 41, and the control section 41 performs identification of the endoscope 2B including no position sensor or the endoscope 2A or 2C including a position sensor based on type information of the endoscope 2I in the identification information.

Then, when the endoscope 2B including no position sensor is used, the image processing section 42 is controlled to estimate the observation position and the sight line direction of the image pickup section 25 or the objective optical system 23 acquired by the UPD apparatus 6 when the endoscope 2A or 2C including a position sensor is used.

In such a case, the image processing section 42 functions as an observation position and sight line direction estimation processing section 42d configured to perform processing of estimating the observation position and the sight line direction (of the image pickup section 25 or the objective optical system 23) of the endoscope 2B by using, for example, a luminance value of two-dimensional endoscope image data as illustrated with a dotted line in FIG. 2. Data on the observation position and the sight line direction estimated by the observation position and sight line direction estimation processing section 42d is stored in a position and direction data storage section 43a provided in a storage region of the information storage section 43. Note that the position of the distal end portion 15 may be estimated in place of the observation position of the image pickup section 25 or the objective optical system 23.

The image processing section 42 includes a 3D shape data structuring section 42a including a CPU, a digital signal processor (DSP), and the like and configured to generate (or structure) 3D shape data (or 3D model data) from two-dimensional endoscope image data inputted from the video processor 4, and an image generation section 42b configured to generate, for the 3D shape data generated (or structured) by the 3D shape data structuring section 42a, a structured region of a 3D model image structured for a two-dimensional image region that is observed (or an image of which is picked up) by the image pickup section 25 of the endoscope and generate a 3D model image that allows (facilitates) visual recognition of an unstructured region of the 3D model image corresponding to a two-dimensional image region unobserved by the image pickup section 25 of the endoscope. In other words, the image generation section 42b generates (or structures) a 3D model image for displaying an unstructured region of the 3D model image in such a manner that allows visual check. The 3D model image generated by the image generation section 42b is outputted to the monitor 8 as a display apparatus and displayed on the monitor 8. The image generation section 42b functions as an output section configured to output a 3D model image (or image of 3D model data) to the display apparatus.

The image processing section 42 includes an image update processing section 42o configured to perform processing of updating, for example, 3D shape data based on change of a region (two-dimensional region corresponding to a three-dimensional region) included in two-dimensional data along with an insertion operation. Note that FIG. 2 illustrates an example in which the image update processing section 42o is provided outside of the image generation section 42b, but the image update processing section 42o may be provided in the image generation section 42b. In other words, the image generation section 42b may include the image update processing section 42o. The image update processing section 42o may be provided to an image processing apparatus in each modification to be described later (not illustrated).

Note that the image processing section 42 and, for example, the 3D shape data structuring section 42a and the image generation section 42b inside the image processing section 42 may be each configured by, in place of a CPU and a DSP, a LSI (large-scale integration) FPGA (field programmable gate array) as hardware configured by a computer program or may be configured by any other dedicated electronic circuit.

The image generation section 42b includes a polygon processing section 42c configured to set, for 3D shape data generated (or structured) by the 3D shape data structuring section 42a, two-dimensional polygons (approximately) expressing each three-dimensional local region in the 3D shape data and perform image processing on the set polygons. Note that FIG. 2 illustrates an exemplary configuration in which the image generation section 42b includes the polygon processing section 42c inside, but it can be effectively regarded that the polygon processing section 42c forms the image generation section 42b.

As described above, when the endoscope 2B including no position sensor is used, the image processing section 42 includes the observation position and sight line direction estimation processing section 42d configured to estimate the observation position and the sight line direction (of the image pickup section 25 or the objective optical system 23) of the endoscope 2B.

The information storage section 43 is configured by, for example, a flash memory, a RAM, a USB memory, or a hard disk apparatus, and includes a position and direction data storage section 43a configured to store view angle data acquired from the memory 30 of the endoscope and store observation position and sight line direction data estimated by the observation position and sight line direction estimation processing section 42d or acquired from the UPD apparatus 6, an image data storage section 43b configured to store, for example, 3D model image data of the image processing section 42, and a boundary data storage section 43c configured to store a structured region of a structured 3D model image and boundary data as a boundary of the structured region.

As illustrated in FIG. 3A, the insertion section 11 of the endoscope 2I is inserted into the ureter 10 having a three-dimensional luminal shape to examine renal pelvis and calyx 51 farther on the deep part side. In this case, the image pickup section 25 disposed at the distal end portion 15 of the insertion section 11 picks up an image of a region in the view angle of the image pickup section 25, and the signal processing circuit 32 generates a two-dimensional image by performing signal processing on image pickup signals sequentially inputted from the image pickup section 25.

Note that the renal pelvis 51a is indicated as a region illustrated with a dotted line in FIG. 3A in the renal pelvis and calyx 51 on the deep part side of the ureter 10, and the renal calyx 51b is located on the deep part side of the renal pelvis 51a.

The 3D shape data structuring section 42a to which two-dimensional image data is inputted generates 3D shape data corresponding to two-dimensional image data picked up (observed) by the image pickup section 25 of the endoscope 2I, by using observation position and sight line direction data acquired by the UPD apparatus 6 or observation position and sight line direction data estimated by the observation position and sight line direction estimation processing section 42d.

In this case, the 3D shape data structuring section 42a may estimate a 3D shape from a corresponding single two-dimensional image by a method disclosed in, for example, the publication of Japanese Patent No. 5354494 or a publicly known shape-from-shading method other than this publication. In addition, a stereo method, a three-dimensional shape estimation method by single-lens moving image pickup, a SLAM method, and a method of estimating a 3D shape in cooperation with a position sensor, which use two images or more are applicable. When a 3D shape is estimated, 3D shape data may be structured with reference to 3D image data acquired from a cross-sectional image acquisition apparatus such as a CT apparatus externally provided.

The following describes a specific method when the image processing section 42 generates 3D model data in accordance with change of (two-dimensional data of) an observation region along with an insertion operation of the endoscope 2I.

The 3D shape data structuring section 42a generates 3D shape data from any region included in a two-dimensional image pickup signal of a subject outputted from the image pickup section 25.

The image update processing section 42o performs processing of updating a 3D model image generated by the 3D shape data structuring section 42a, based on change of two-dimensional data along with the insertion operation of the endoscope 2I.

More specifically, for example, when a first two-dimensional image pickup signal generated at the image pickup section 25 upon reception of return light from a first region in the subject is inputted, the 3D shape data structuring section 42a generates first 3D shape data corresponding to the first region included in the first two-dimensional image pickup signal. The image update processing section 42o stores the first 3D shape data generated by the 3D shape data structuring section 42a in the image data storage section 43b.

When a second two-dimensional image pickup signal generated at the image pickup section 25 upon reception of return light from a second region different from the first region is inputted after the first 3D shape data is stored in the image data storage section, the 3D shape data structuring section 42a generates second 3D shape data corresponding to the second region included in the second two-dimensional image pickup signal. The image update processing section 42o stores, in addition to the first 3D shape data, the second 3D shape data generated by the 3D shape data structuring section 42a in the image data storage section 43b.

Then, the image update processing section 42o generates a current 3D model image by synthesizing the first 3D shape data and the second 3D shape data stored in the image data storage section 43b, and outputs the generated 3D model image to the monitor 8.

Thus, when the distal end portion 15 of the endoscope 2I is moved by the insertion operation, a 3D model image corresponding to any region included in an endoscope image observed in the past from start of the 3D model image generation to the current observation state of the distal end portion 15 is displayed on the monitor 8. The display region of the 3D model image displayed on the monitor 8 increases with time elapse.

Note that, when a 3D model image is displayed on the monitor 8 by using the image update processing section 42o, a (second) 3D model image corresponding only to a structured region that is already observed can be displayed, but convenience can be improved for the user by displaying instead a (first) 3D model image that allows visual recognition of a region yet to be structured. Thus, the following description will be mainly made on an example in which the (first) 3D model image that allows visual recognition of an unstructured region is displayed.

The image update processing section 42o updates the (first) 3D model image based on change of a region included in endoscope image data as inputted two-dimensional data. The image update processing section 42o compares inputted current endoscope image data with endoscope image data used to generate the (first) 3D model image right before the current endoscope image data.

Then, when a detected change amount is equal to or larger than a threshold set as a comparison result in advance, the image update processing section 42o updates the past (first) 3D model image with the (first) 3D model image based on the current endoscope image data.

Note that, when updating the (first) 3D model image, the image update processing section 42o may use, for example, information on a leading end position of the endoscope 2I, which changes along with the insertion operation of the endoscope 2I. To achieve such processing, for example, the image processing apparatus 7 may be provided with a position information acquisition section 81 as illustrated with a dotted line in FIG. 2.

The position information acquisition section 81 acquires leading end position information as information indicating the leading end position of the distal end portion 15 of the insertion section 11 of the endoscope 2I, and outputs the acquired leading end position information to the image update processing section 42o.

The image update processing section 42o determines whether the leading end position in accordance with the leading end position information inputted from the position information acquisition section 81 has changed from a past position. Then, when having acquired a determination result that the leading end position in accordance with the leading end position information inputted from the position information acquisition section 81 has changed from the past position, the image update processing section 42o generates the current (first) 3D model image including a (first) 3D model image part based on two-dimensional data inputted at a timing at which the determination result is acquired. Namely, the image update processing section 42o updates the (first) 3D model image before the change with a (new first) 3D model image (after the change).

The respective barycenters of the (first) 3D model image and the past (first) 3D model image may be calculated, and the update may be performed when a detected change amount is equal to or larger than a threshold set as a comparison result in advance.

Alternatively, information used by the image update processing section 42o when updating the (first) 3D model image may be selected from among two-dimensional data, a leading end position, and a barycenter in accordance with, for example, an operation of the input apparatus 44 by the user, or all of the two-dimensional data, the leading end position, and the barycenter may be selected. That is, the input apparatus 44 functions as a selection section configured to allow selection of at least one of two pieces (or two kinds) of information used by the image update processing section 42o when updating the (first) 3D model image.

The present endoscope system includes the endoscope 2I configured to observe inside of a subject having a three-dimensional shape, the signal processing circuit 32 of the video processor 4 serving as an input section configured to input two-dimensional data of (the inside of) the subject observed by the endoscope 2I, the 3D shape data structuring section 42a or the image generation section 42b serving as a three-dimensional model image generation section configured to generate a three-dimensional model image that represents the shape of the subject and is to be outputted to the monitor 8 as a display section based on a region included in the two-dimensional data of the subject inputted by the input section, and the image update processing section 42o configured to update the three-dimensional model image to be outputted to the display section based on change of the region included in the two-dimensional data along with an insertion operation of the endoscope 2I and output the updated three-dimensional model image to the display section.

Besides processing of storing the first 3D shape data and the second 3D shape data in the image data storage section 43b, generating a 3D model image, and outputting the generated 3D model image to the monitor 8, the image update processing section 42o may also be configured to output a 3D model image generated by performing any processing other than the processing to the monitor 8.

More specifically, the image update processing section 42o may perform, for example, processing of storing only the first 3D shape data in the image data storage section 43b, generating a 3D model image by synthesizing the first 3D shape data read from the image data storage section 43b and the second 3D shape data inputted after the first 3D shape data is stored in the image data storage section 43b, and outputting the generated 3D model image to the monitor 8. Alternatively, the image update processing section 42o may perform, for example, processing of generating a 3D model image by synthesizing the first 3D shape data and the second 3D shape data without storing the first 3D shape data and the second 3D shape data in the image data storage section 43b, storing the 3D model image in the image data storage section 43b, and outputting the 3D model image read from the image data storage section 43b to the monitor 8.

Alternatively, the image update processing section 42o is not limited to storage of 3D shape data generated by the 3D shape data structuring section 42a in the image data storage section 43b, but may store, in the image data storage section 43b, a two-dimensional image pickup signal generated at the image pickup section 25 when return light from the inside of the subject is received.

More specifically, for example, when the first two-dimensional image pickup signal generated at the image pickup section 25 upon reception of return light from the first region in the subject is inputted, the image update processing section 42o stores the first two-dimensional image pickup signal in the image data storage section 43b.

When the second two-dimensional image pickup signal generated at the image pickup section 25 upon reception of return light from the second region different from the first region is inputted after the first two-dimensional image pickup signal is stored in the image data storage section 43b, the image update processing section 42o stores, in addition to the first two-dimensional image pickup signal, the second two-dimensional image pickup signal in the image data storage section 43b.

Then, the image update processing section 42o generates a three-dimensional model image corresponding to the first region and the second region based on the first image pickup signal and the second image pickup signal stored in the image data storage section 43b, and outputs the three-dimensional model image to the monitor 8.

The following describes a display timing that is a timing at which the image update processing section 42o outputs the three-dimensional model image corresponding to the first region and the second region to the monitor 8.

For example, at each predetermined duration (for example, every second), the image update processing section 42o updates 3D shape data stored in the image data storage section 43b and outputs the updated 3D shape data to the monitor 8. Then, according to such processing by the image update processing section 42o, a three-dimensional model image corresponding to a two-dimensional image pickup signal of the inside of an object sequentially inputted to the image processing apparatus 7 can be displayed on the monitor 8 while being updated.

Note that, for example, when a trigger signal as a trigger for updating an image is inputted in response to an operation of the input apparatus 44 by the user, the image update processing section 42o may update 3D shape data stored in the image data storage section 43b at each predetermined duration (for example, every second), generate a three-dimensional model image in accordance with the 3D shape data, and output the three-dimensional model image to the monitor 8. According to such processing by the image update processing section 42o, the three-dimensional model image can be displayed on the monitor 8 while being updated at a desired timing, and thus convenience can be improved for the user.

For example, when having sensed that no treatment instrument such as a basket is present in an endoscope image corresponding to a two-dimensional image pickup signal generated by the image pickup section 25 (namely, when having sensed that the endoscope is inserted in a pipe line, not in treatment of a lesion site), the image update processing section 42o may output the three-dimensional model image to the monitor 8 while updating the three-dimensional model image.

According to the processing as described above, for example, a 3D model image displayed (in a display region adjacent to an endoscope image) on the monitor 8 is updated in the following order of I3oa in FIG. 3B, I3ob in FIG. 3C, and I3oc in FIG. 3D in response to change of (two-dimensional data of) the observation region along with an insertion operation of the endoscope 2I inserted into renal pelvis and calyx.

The 3D model image I3oa illustrated in FIG. 3B is an image generated based on an endoscope image observed up to an insertion position illustrated on the right side in FIG. 3B. An upper end part in the 3D model image I3oa is a boundary Ba between a structured region corresponding to an observation region that is observed and an unobserved region, and the boundary Ba portion is displayed in a color different from the color of the structured region.

Note that an arrow in the 3D model image I3oa illustrated in FIG. 3B indicates the position and the direction of the distal end portion 15 of the endoscope 2A (same in FIGS. 3C and 3D). The above-described arrow as an index indicating the position and the direction of the distal end portion 15 of the endoscope 2A may be superimposed in the 3D model image I3oa.

The 3D model image I3ob illustrated in FIG. 3C is a 3D model image updated by adding a structured region to an unstructured region part in the 3D model image I3oa illustrated in FIG. 3B.

In the 3D model image I3ob illustrated in FIG. 3C, boundaries Bb, Bc, and Bd with a plurality of unstructured regions are generated due to bifurcation parts halfway through the insertion. Note that the boundary Bd includes a part not attributable to a bifurcation part.

The 3D model image I3oc illustrated in FIG. 3D is a 3D model image updated by adding a structured region to an unstructured region on an upper part side in the 3D model image I3ob illustrated in FIG. 3C.

In the present embodiment, the insertion section 11 of the endoscope 2I is inserted through the ureter 10 having a luminal shape into the renal pelvis and calyx 51 having a luminal shape on the deep part side of the ureter 10. In this case, the 3D shape data structuring section 42a structures hollow 3D shape data when the inner surface of the organ having a luminal shape is observed.

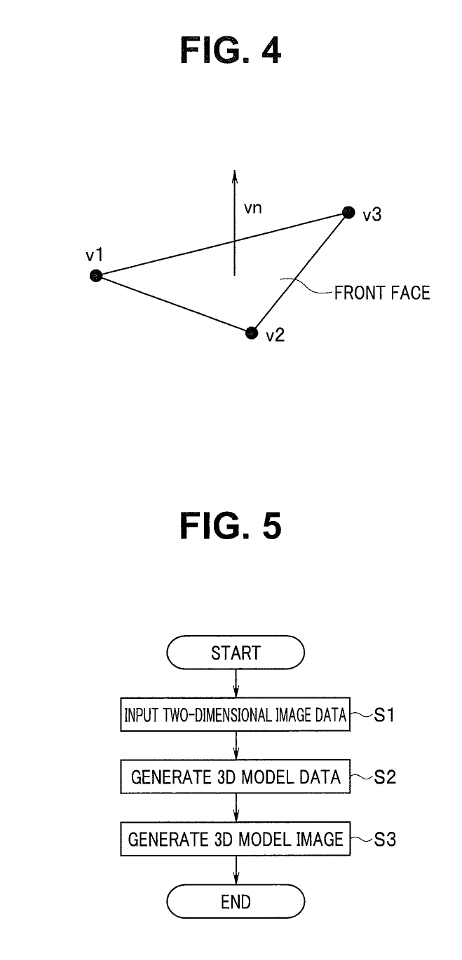

The image generation section 42b (the polygon processing section 42c) sets polygons to the 3D shape data structured by the 3D shape data structuring section 42a and generates a 3D model image using the polygons. In the present embodiment, the 3D model image is generated by performing processing of bonding triangles as polygons onto the surface of the 3D shape data. That is, the 3D model image employs triangular polygons as illustrated in FIG. 4. Typically, triangles or rectangles are often used as polygons, but in the present embodiment, triangular polygons are used. Note that the 3D shape data structuring section 42a may directly generate (or structure) a 3D model image instead of the 3D shape data.

Each polygon can be disassembled into a plane, sides, and apexes, and each apex is described with 3D coordinates. The plane has front and back surfaces, and one perpendicular normal vector is set to the plane.

The front surface of the plane is set by the order of description of the apexes of the polygon. For example, as illustrated in FIG. 4, the front and back surface (of the plane) when described in the following order of three apexes v1, v2, and v3 correspond to the direction of a normal vector vn.

As described later, the setting of a normal vector corresponds to determination of the front and back surfaces of a polygon to which the normal vector is set, in other words, determination of whether each polygon on a 3D model image (indicating an observed region) formed by using the polygons corresponds to the inner surface (or inner wall) or the outer surface (or outer wall) of the luminal organ. In the present embodiment, it is a main objective to observe or examine the inner surface of the luminal organ, and thus the following description will be made on an example in which the inner surface of the luminal organ is associated with the front surface of the plane of each polygon (the outer surface of the luminal organ is associated with the back surface of the plane of the polygon). When the inner and outer surfaces of a luminal structural body in a subject having a more complicated shape and including the luminal structural body inside are examined, the present embodiment is also applicable to the complicated subject to distinguish (determine) the inner and outer surfaces.

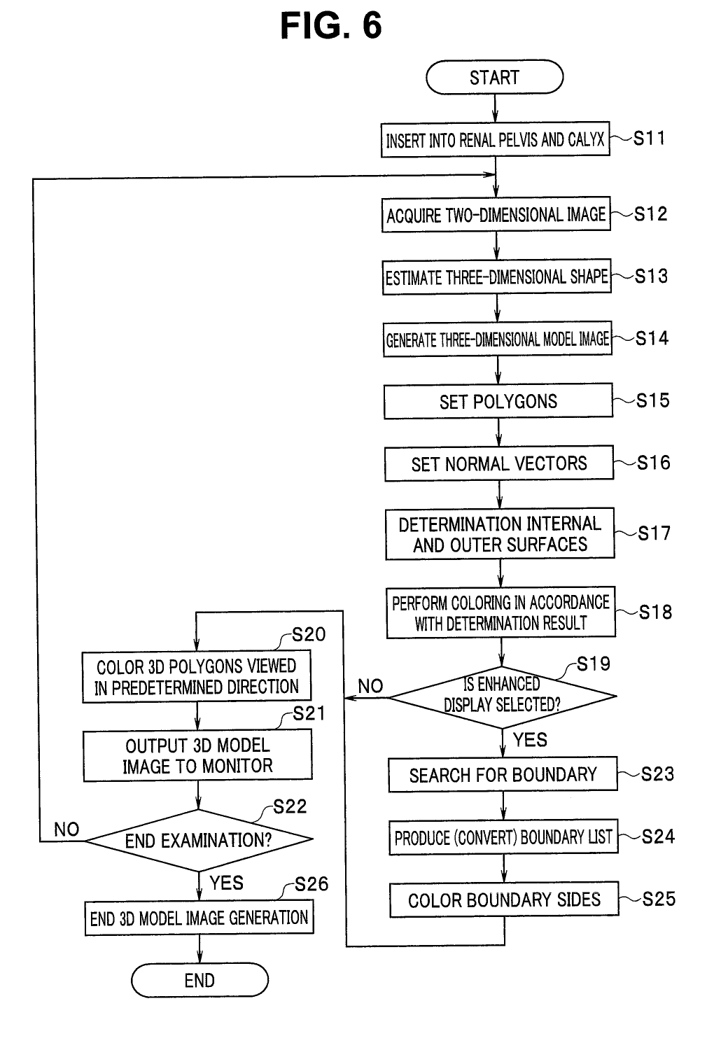

Note that, as described later with reference to FIG. 6, each time when the insertion position of the insertion section 11 moves and a region of a two-dimensional image acquired through observation by the image pickup section 25 changes, the image processing section 42 repeats processing of generating 3D shape data of the changed region, updating 3D shape data before the change with the generated 3D shape data, newly setting a polygon on the updated region appropriately by using the normal vector, and generating a 3D model image through addition (update).

The image generation section 42b functions as an inner and outer surface determination section 42e configured to determine, when adding a polygon, whether an observed local region represented by the plane of the polygon corresponds to the inner surface (inner wall) or the outer surface (outer wall) by using the normal vector of the polygon.

When enhanced display in which a boundary is displayed in an enhanced manner is selected through the enhanced display selection section 44b of the input apparatus 44, the image generation section 42b functions as a boundary enhancement processing section 42f configured to display, in an enhanced manner, a boundary region with a structured region (as an observed and structured region) (the boundary region also serves as a boundary with an unstructured region as a region yet to be observed and structured) in the 3D model image. The boundary enhancement processing section 42f does not perform the processing of enhancing a boundary region (boundary part) when the enhanced display is not selected through the enhanced display selection section 44b by the user.

In this manner, when a 3D model image is displayed on the monitor 8, the user can select the enhanced display of a boundary with an unstructured region to facilitate visual recognition or select display of the 3D model image on the monitor 8 without selecting the enhanced display.

The image generation section 42b includes a (polygon) coloring processing section 42g configured to color, in different colors, the inner and outer surfaces of the plane of a structured (in other words, observed) polygon with which a 3D model image is formed, in accordance with a determination result of inner and outer surfaces. Note that different textures may be attached to a polygon instead of the coloring in different colors. The following description will be made on an example in which the display color setting section 44a is set to color an inner surface (observed) in gray and an outer surface (unobserved) in white. Gray may be set to be close to white. The present embodiment is not limited to the example in which the inner surface is colored in gray and the outer surface is colored in white (the coloring is performed by the coloring processing section 42g corresponding to a color set by the display color setting section 44a).

Note that, in the present embodiment, in a normal observation mode in which the inner surface of the luminal organ is an observation target, an unobserved region is the inner surface of the luminal organ, an image of which is yet to be picked up by the image pickup section 25.

Then, when the unobserved region is displayed on a 3D model image to allow visual recognition by the operator, for example, during observation and examination with the endoscope 2I, any unstructured region existing on the 3D model image and corresponding to the unobserved region can be displayed in an image that allows easy visual recognition in a 3D space by displaying the 3D model image in a shape close to the shape of the renal pelvis and calyx 51 illustrated in FIG. 3A.

Thus, in the present embodiment, the image processing section 42 generates, by using polygons, a 3D model image of the renal pelvis and calyx 51 as a luminal organ illustrated in FIG. 3A when viewed from a viewpoint vertically above the sheet of the FIG. 3A in a predetermined direction.

When the viewpoint is set outside of the luminal organ in this manner, it is difficult to display an actually observed region existing on the inner surface of a lumen in a manner that allows easy visual recognition as an observed structured region on a 3D model image viewed from a viewpoint set on the outer surface of the lumen.

The difficulty can be avoided as described in the following methods (a), (b), and (c). The methods (a) and (b) are applicable to a double (or multiplex) tubal structure, and the method (c) is applicable to a single tubal structure such as a renal pelvis.

(a) When a (drawn) 3D model image is viewed from a viewpoint, a region of the outer surface covering an observed structured region on the 3D model image is colored in a display color (for example, green) different from gray as the color of the inner surface and white as the color of the outer surface. (b) As illustrated with a double-dotted and dashed line in FIG. 3A, for example, an illumination light source Ls is set at a viewpoint at a position vertically above the sheet of FIG. 3A, and an outer surface region covering a structured region on a 3D model image observed with illumination light radially emitted from the light source Ls may be displayed in a display color (for example, green) colored in the color of the illumination light of the illumination light source Ls.

(c) In a limited case in which only the inner surface of the luminal organ is an observation target, the outer surface of the luminal organ is not an observation target, and thus when the outer surface covers the observed inner surface of the luminal organ, the outer surface may be displayed in a display color different from gray as the color of the inner surface. In such a case, white may be set as a display color in which the observed inner surface covered by the outer surface is displayed. In the following, a display color different (or easily distinguishable) at least from gray (as a color in which the inner surface observed and not covered by the outer surface is displayed in a direct manner (in an exposing manner)) is used as a display color in which the outer surface when covering the observed inner surface of the luminal organ is displayed. In the present specification, the outer surface covering the observed inner surface is displayed in this manner in a display color different from color (for example, gray) when the observed inner surface is observed directly in an exposed state.

In the present embodiment, a background part of a 3D model image is set to have a background color (for example, blue) different from a color (gray) in which the observed inner surface is displayed in display of the 3D model image and the display color (for example, green) of the outer surface when the observed inner surface is covered by the outer surface in a double tubal structure, thereby achieving easy visual recognition (display) of a boundary region as a boundary between a structured region and an unstructured region together with an observed structured region. When the enhanced display is selected, the coloring processing section 42g colors the boundary region in a color (for example, red) different from gray, the display color, and the background color for easier visual recognition.

Note that, in FIG. 1, the image processing apparatus 7 is provided separately from the video processor 4 and the light source apparatus 3 included in the endoscope apparatus, but the image processing apparatus 7 may be provided in the same housing of the video processor 4 and the light source apparatus 3.

The endoscope system 1 according to the present embodiment includes the endoscope 2I configured to observe the inside of the ureter 10 or the renal pelvis and calyx 51 as a subject having a three-dimensional shape, the signal processing circuit 32 of the video processor 4 serving as an input section configured to input two-dimensional data of (the inside of) the subject observed by the endoscope 2I, the 3D shape data structuring section 42a serving as a three-dimensional model structuring section configured to generate (or structure) three-dimensional model data or three-dimensional shape data of the subject based on the two-dimensional data of the subject inputted by the input section, and the image generation section 42b configured to generate a three-dimensional model image that allows visual recognition of an unstructured region (in other words, that facilitates visual recognition of the unstructured region or in which the unstructured region can be visually recognized) as an unobserved region in the subject based on the three-dimensional model data of a structured region, which is structured by the three-dimensional model structuring section.

As illustrated in FIG. 5, an image processing method in the present embodiment includes: an input step S1 at which the signal processing circuit 32 of the video processor 4 inputs, to the image processing apparatus 7, two-dimensional image data as two-dimensional data of (the inside of) a subject observed by the endoscope 2I configured to observe the inside of the ureter 10 or the renal pelvis and calyx 51 as a subject having a three-dimensional shape; a three-dimensional model structuring step S2 at which the 3D shape data structuring section 42a generates (or structures) three-dimensional model data (3D shape data) of the subject based on the two-dimensional data (2D data) of the subject inputted at the input step S1; and an image generation step S3 at which the image generation section 42b generates, based on the three-dimensional model data of a structured region structured at the three-dimensional model structuring step S2, a three-dimensional model image that allows visual recognition of an unstructured region (in other words, that facilitates visual recognition of the unstructured region or in which the unstructured region can be visually recognized) as an unobserved region in the subject. Note that contents of processing illustrated in FIG. 5 outline contents of processing illustrated in FIG. 6 to be described below.

The following describes an operation according to the present embodiment with reference to FIG. 6. FIG. 6 illustrates the procedure of main processing by the endoscope system 1 according to the present embodiment. Note that, in the processing illustrated in FIG. 6, different system configurations and image processing methods may be employed between a case in which the enhanced display is not selected and a case in which the enhanced display is selected.

As illustrated in FIG. 1, the operator connects the image processing apparatus 7 to the light source apparatus 3 and the video processor 4 and connects the endoscope 2A, 2B, or 2C to the light source apparatus 3 and the video processor 4 before performing an endoscope examination. In the examination, the insertion section 11 of the endoscope 2I is inserted into the ureter 10 of the patient 9. Then, as described at step S11 in FIG. 6, the insertion section 11 of the endoscope 2I is inserted into the renal pelvis and calyx 51 on the deep part side through the ureter 10 as illustrated in FIG. 3A.

The image pickup section 25 is provided at the distal end portion 15 of the insertion section 11 and inputs an image pickup signal picked up (observed) in the view angle of the image pickup section 25 to the signal processing circuit 32 of the video processor 4.

As described at step S12, the signal processing circuit 32 performs signal processing on the image pickup signal picked up by the image pickup section 25 to generate (acquire) a two-dimensional image observed by the image pickup section 25. The signal processing circuit 32 inputs (two-dimensional image data obtained through A/D conversion of) the generated two-dimensional image to the image processing section 42 of the image processing apparatus 7.

As described at step S13, the 3D shape data structuring section 42a of the image processing section 42 generates 3D shape data from the inputted two-dimensional image data by using information of a position sensor when the endoscope 2A (or 2C) including the position sensor is used or by performing image processing to estimate a 3D shape corresponding to an image region observed (by the image pickup section 25) and estimating 3D shape data as 3D model data when the endoscope 2B including no position sensor is used.

The 3D shape data may be generated from the two-dimensional image data by the method described above.

At the next step S14, the image generation section 42b generates a 3D model image by using polygons. As illustrated in FIG. 6, similar processing is repeated in a loop. Thus, at the second repetition or later, the processing at step S14 continues the processing of generating a 3D model image by using polygons at the last repetition (generating a 3D model image for any new polygon and updating the previous 3D model image).

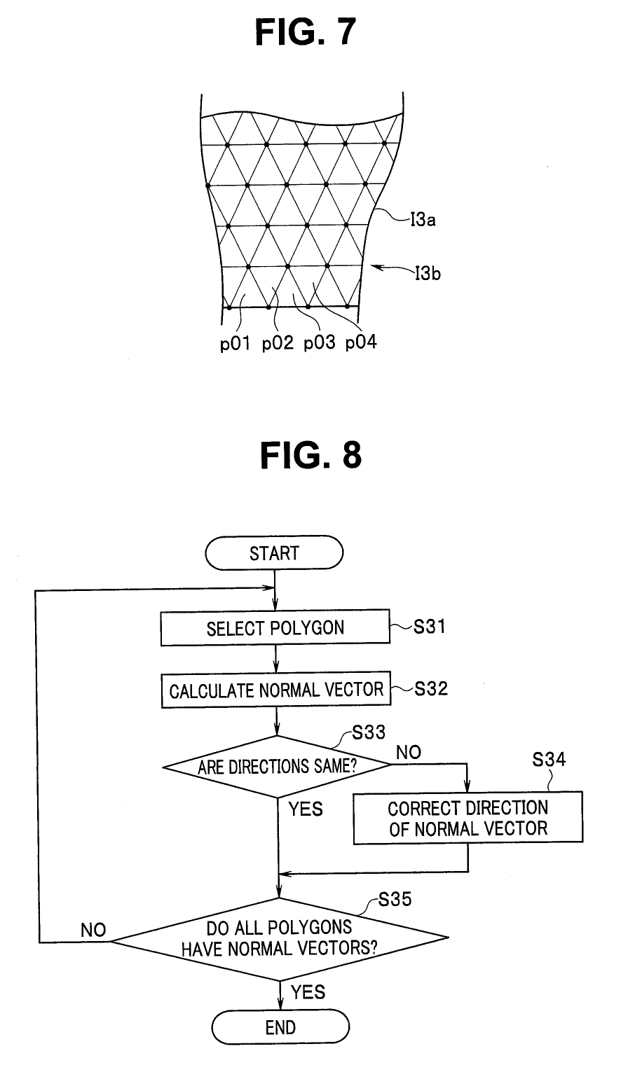

At the next step S15, the polygon processing section 42c generates polygons by a well-known method such as the method of marching cubes based on the 3D shape data generated at step S13. FIG. 7 illustrates a situation in which polygons are generated based on the 3D shape data generated at step S13.

In 3D shape data (an outline shape part in FIG. 7) I3a generated to illustrate a lumen, polygons are set onto the outer surface of the lumen when the lumen is viewed from a side, thereby generating a 3D model image I3b.

Note that a 3D model image I3c is then generated through coloring processing and displayed on the monitor 8. FIG. 7 illustrates polygons pO1, pO2, pO3, pO4, and the like.

At the next step S16, the polygon processing section 42c sets a normal vector to each polygon set at the previous step S15 (to determine whether an observed region is an inner surface).

At the next step S17, the inner and outer surface determination section 42e of the image generation section 42b determines whether the observed region is an inner surface by using the normal vector. Processing at steps S16 and S17 will be described later with reference to FIG. 8.

At the next step S18, the coloring processing section 42g of the image generation section 42b colors the plane of each polygon representing the observed region (in gray for the inner surface or white for the outer surface) in accordance with a determination result at the previous step S17.

At the next step S19, the control section 41 (or the boundary enhancement processing section of the image generation section 42b) determines whether the enhanced display is selected. When the enhanced display is not selected, the process proceeds to processing at the next step S20. The next step S20 is followed by processing at steps S21 and S22.

When the enhanced display is selected, the process performs processing at steps S23, S24, and S25, and then proceeds to the processing at step S20.

At step S20, the coloring processing section 42g of the image generation section 42b colors an observed surface of a polygon in a structured region of the 3D model image when viewed (at a position set outside of or separately from the 3D model image) in a predetermined direction is an inner surface, in a color corresponding to a case in which the plane is hidden behind the outer surface.

Similarly to the double tubal structure described above, when an observed surface of a polygon in a structured region of the 3D model image viewed in a predetermined direction is an inner surface and a 3D model image in which the inner surface is covered by the outer surface is displayed, the outer surface is colored in a display color (for example, green) different from gray as a display color indicating an observed inner surface, white as the color of an observed outer surface, and the background color. Note that, when the 3D model image is displayed, an observed inner surface being exposed remains in gray, which is provided in the coloring processing at step S18.

At step S21 following the processing at step S20, the image processing section 42 or the image generation section 42b outputs an image signal of the 3D model image generated (by the above-described processing) to the monitor 8, and the monitor 8 displays the generated 3D model image.

At the next step S22, the control section 41 determines whether the operator inputs an instruction to end the examination through, for example, the input apparatus 44.

When the instruction to end the examination is not inputted, the process returns to the processing at step S11 or step S12 and repeats the above-described processing. That is, when the insertion section 11 is moved in the renal pelvis and calyx 51, the processing of generating 3D shape data corresponding to a region newly observed by the image pickup section 25 after the movement and generating a 3D model image for the 3D shape data is repeated.

When the instruction to end the examination is inputted, the image processing section 42 ends the processing of generating a 3D model image as described at step S26, which ends the processing illustrated in FIG. 6.

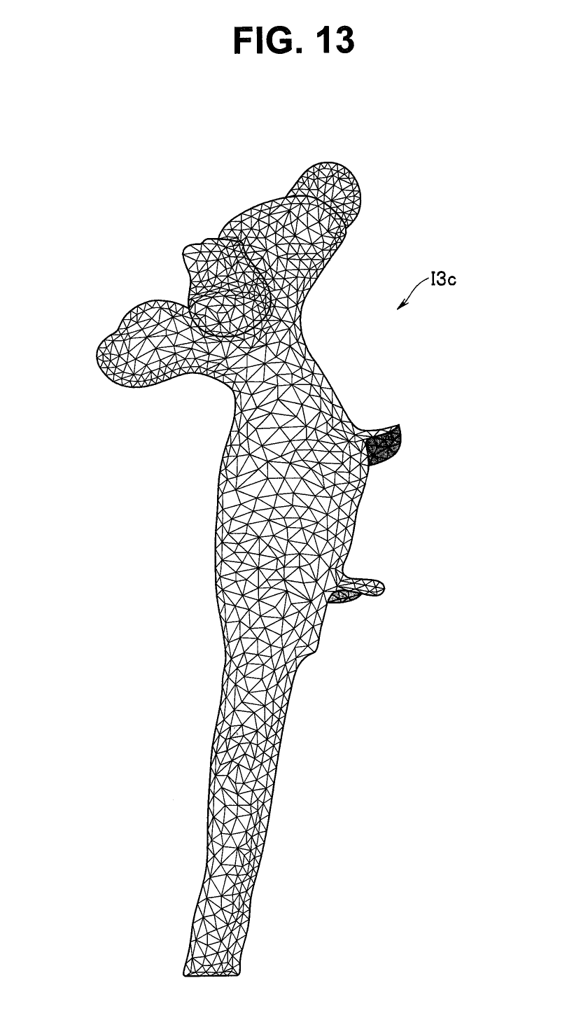

FIG. 13 illustrates the 3D model image I3c displayed on the monitor 8 halfway through the repetition of the above-described processing, for example, after the processing at step S21 when the enhanced display is not selected (when the processing at steps S23, S24, and S25 are not performed).

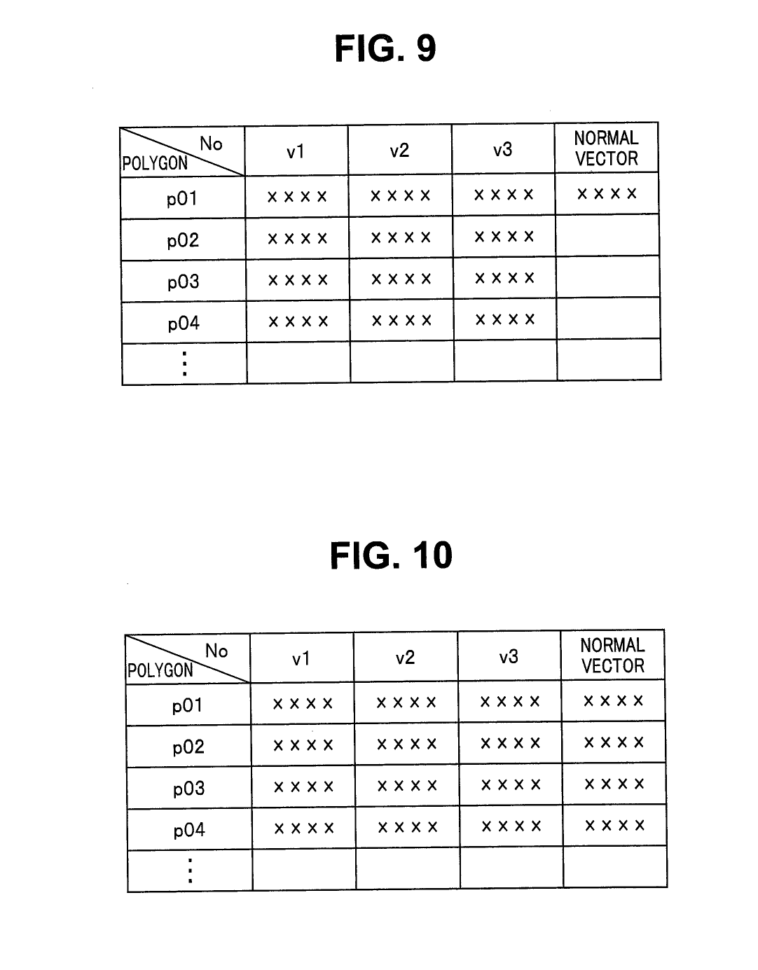

The processing at steps S16 and S17 in FIG. 6 will be described next with reference to FIG. 8. Through the processing at step S15, as illustrated in FIG. 7, a plurality of polygons pO1, pO2, pO3, pO4, and the like are set to the 3D shape data 13a of the observed region. These polygons pj (j=01, 02, 03, . . . ) are stored (held) as a polygon list in a table format illustrated in FIG. 9 in the information storage section 43. The three apexes v1, v2, and v3 of each polygon pj are each determined with a three-dimensional position vector value XXXX. Note that the polygon list indicates the configuration of each polygon.

At the first step S31 in FIG. 8, the polygon processing section 42c selects a polygon. As illustrated in FIG. 9, the polygon pO2 adjacent to the polygon pO1 to which a normal vector indicated with XXXX is set is selected. Note that, a normal vector vn1 of the polygon pO1 is set in the direction of a front surface indicating an observed inner surface as described with reference to FIG. 4.

At the next step S32, the polygon processing section 42c calculates a normal vector vn2 of the polygon pO2 as vn2=(v2−v1)×(v3−v1). Note that, to simplify description, the three-dimensional positions of the apexes v1, v2, and v3 are represented by using v1, v2, and v3, and, for example, v2−v1 represents a vector extending from the three-dimensional position v1 to the three-dimensional position v2.

At the next step S33, the polygon processing section 42c determines whether the direction (or polarity) of the normal vector vn2 of the polygon pO2 is same as the registered direction of the normal vector vn1 of the polygon pO1.

To perform the determination, the polygon processing section 42c calculates the inner product of the normal vector vn1 of the polygon pO1 adjacent to the polygon pO2 at an angle equal to or larger than 90 degrees and the normal vector vn2 of the polygon pO2, and determines that the directions are same when the value of the inner product is equal to or larger than zero, or determines that the directions are inverted with respect to each other when the value is less than zero.

When it is determined that the directions are inverted with respect to each other at step S33, the polygon processing section 42c corrects the direction of the normal vector vn2 at the next step S35. For example, the normal vector vn2 is corrected by multiplication by −1 and registered, and the position vectors v2 and v3 in the polygon list are swapped.

After step S34 or when it is determined that the directions are same at step S33, the polygon processing section 42c determines whether all polygons have normal vectors (normal vectors are set to all polygons) at step S35.

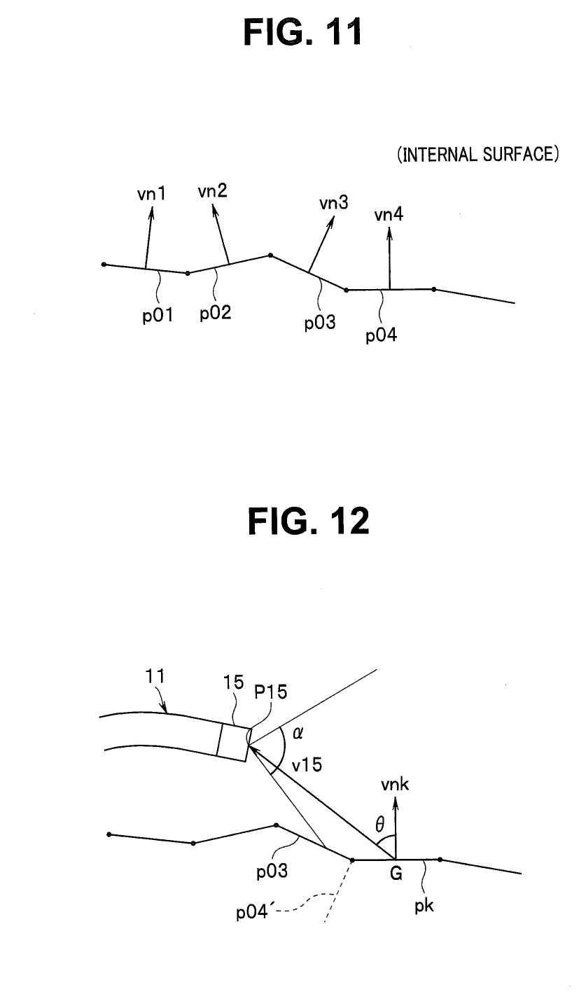

The process returns to the processing at the first step S31 when there is any polygon having no normal vector, or the processing illustrated in FIG. 8 is ended when all polygons have normal vectors. FIG. 10 illustrates a polygon list obtained by setting normal vectors to the polygon list illustrated in FIG. 9. FIG. 11 illustrates a situation in which, for example, the normal vector vn2 is set to the polygon pO2 adjacent to the polygon pO1 through the processing illustrated in FIG. 8. Note that, in FIG. 11, the upper part sides of the polygons 02 to 04 correspond to the inner surface of the luminal organ (and the lower sides correspond to the outer surface).

In the above description, whether the directions of normal vectors are same is determined by using an inner product in the determination processing at step S33 in FIG. 8. This method is also applicable to the endoscope 2B including no position sensor.

However, when the endoscope 2A (or 2C) including a position sensor at the distal end portion 15 is used, information of the position sensor as illustrated in FIG. 12 may be used to determine whether the direction of a normal vector is same as the direction of a registered adjacent normal vector.

The inner product of a vector v15 connecting the barycenter G of a polygon pk as a determination target and the position p15 of the distal end portion 15 when a two-dimensional image used in 3D shape estimation is acquired as illustrated in FIG. 12 and the normal vector vnk of the polygon pk is calculated, and it is determined that the directions are same when the value of the inner product is equal to or larger than zero, or it is determined that the directions are inverted with respect to each other when the value is less than zero. In FIG. 12, an angle θ between both vectors is smaller than 90°, and the inner product is equal to or larger than zero.

Accordingly, in FIG. 12, for example, the inner surface of a polygon pO4′ at an obtuse angle with respect to the inner surface of an adjacent polygon (pO3 in FIG. 12) as illustrated with a dotted line cannot be observed (thus, such a polygon is not generated, and the determination of the direction of a normal vector is not performed).

In this manner, when the enhanced display is not selected, the 3D model image I3b as illustrated in FIG. 13 is displayed on the monitor 8 in a color different from a background color.

Most of a luminal organ extending from the ureter on the lower side to the renal pelvis and calyx on the upper side is drawn with polygons (whereas part of the luminal organ lacks) as illustrated in FIG. 13, and the (outer) plane of a polygon representing the outer surface of the luminal organ is displayed in a whitish color (for example, green). Note that the surround of polygons in the 3D model image I3c is displayed in a background color such as blue.

In FIG. 13, part of the inner surface colored in gray is displayed at a lower renal calyx part, and the inner surface colored in gray is displayed at a middle renal calyx part above the lower renal calyx part. A boundary is exposed in an upper renal calyx in FIG. 13.

The operator can easily visually recognize, from the 3D model image I3c in which the inner surface is displayed in a predetermined color in this manner with a boundary region at the inner surface colored in the predetermined color, that an unstructured region is not structured nor colored because the region is yet to be observed exists.

In this manner, the 3D model image I3c displayed as illustrated in FIG. 13 is a three-dimensional model image displayed in such a manner that the operator can easily visually recognize any unstructured region.

Note that, when the 3D model image I3c as illustrated in FIG. 13 is generated, a partial region of the inner surface which cannot be observed typically from outside of a closed luminal organ is displayed in a color that allows easy visual recognition, and thus it can be visually recognized that a region adjacent to the region is an unstructured region that is not observed.

However, like, for example, the upper renal calyx in FIG. 13, when an observed inner surface is hidden behind an outer surface in front of the inner surface and is not displayed, and has a boundary shape difficult to visually recognize opening of the boundary, it is potentially overlooked that an unstructured region exists in the part. The operator understands the shape of the luminal organ on which observation or examination is performed, and thus the probability of overlooking is low, but it is desired to reduce a load on the operator as much as possible to allow the operator to easily and smoothly perform endoscope examination.

In the present embodiment, the enhanced display can be selected to achieve the reduction, and processing at steps S23, S24, and S25 in FIG. 6 is performed when the enhanced display is selected.

When the enhanced display is selected, the boundary enhancement processing section 42f performs processing of searching for (or extracting) a side of a polygon in a boundary region by using information of a polygon list at step S23.

When the luminal organ as an examination target is the renal pelvis and calyx 51, the renal pelvis 51a bifurcates into a plurality of the renal calyces 51b. In the example illustrated in FIG. 7, three sides of each polygon pi are each shared with an adjacent polygon.

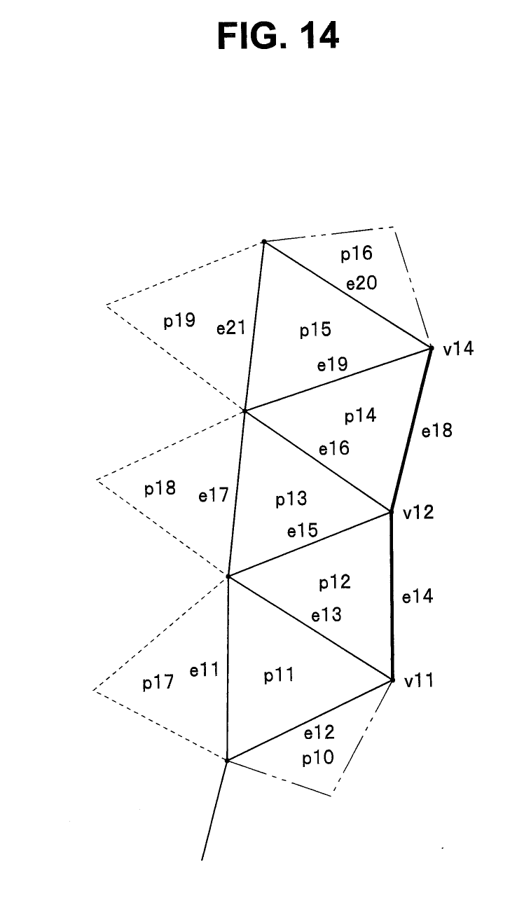

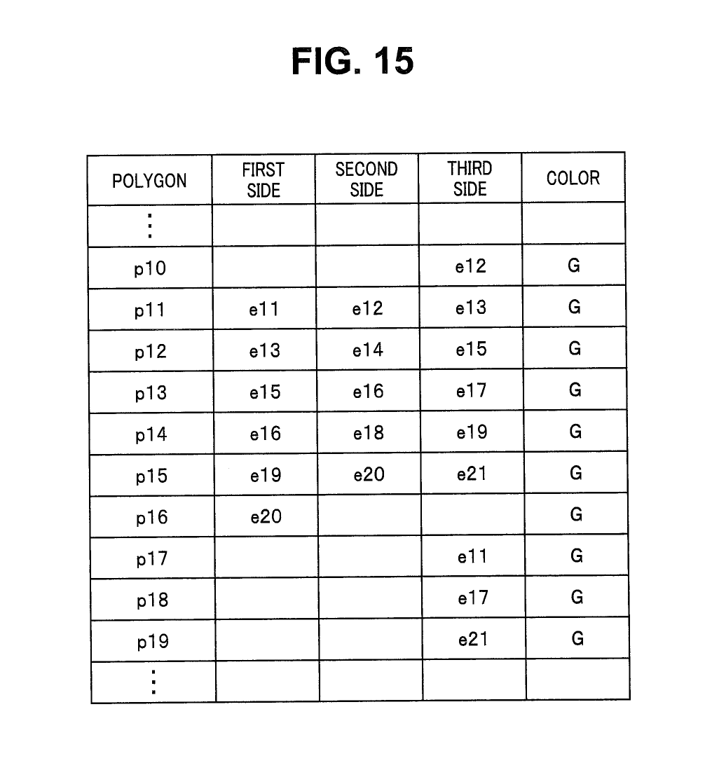

However, a polygon at an edge of a structured region and in a boundary region with an unstructured region has a side not shared with any other polygon. FIG. 14 schematically illustrates polygons near a boundary, and FIG. 15 illustrates a polygon list corresponding to the polygons illustrated in FIG. 14.

In FIG. 14, a side e14 of a polygon p12 and a side e18 of a polygon p14 indicate a boundary side, and the right side of the sides is an unstructured region. In FIG. 14, the boundary side is illustrated with a bold line. In reality, the boundary side typically includes a larger number of sides. Note that, in FIG. 14, sides e11, e17, and e21 are shared between polygons p11, p13, and p15 and polygons p17, p18, and p19 illustrated with dotted lines. Sides e12 and e20 are shared between the polygons p11 and p15 and polygons p10 and p16 illustrated with double-dotted and dashed lines.

In the example illustrated in FIG. 14, the polygon list as illustrated in FIG. 15 is obtained, and in the polygon list, the side e14 of the polygon p12 and the side e18 of the polygon p14 appear once, and the other sides appear twice. Thus, the polygon processing section 42c extracts, as a boundary side, any side appearing once from the polygon list in the processing of searching for (a polygon of) a boundary region. In other words, the polygon processing section 42c extracts, as a boundary side, a side not shared between a plurality of polygons (three-dimensionally adjacent to each other) (that is, a side belonging to only one polygon) in a polygon list as a list of all polygons representing an observed structured region.

Note that a color used in coloring in accordance with a determination result of whether an observed plane of a polygon is an inner surface or an outer surface is set in the rightmost column in the polygon list illustrated in FIG. 15. In FIG. 15, since the inner surface is observed, G representing gray is set.

At the next step S24, the boundary enhancement processing section 42f produces a boundary list from the information extracted at the previous step S23 and notifies the coloring processing section 42g of the production.

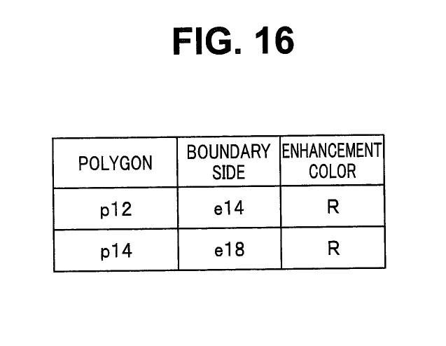

FIG. 16 illustrates the boundary list generated at step S24. The boundary list illustrated in FIG. 16 is a list of a boundary side of any polygon searched for (extracted) up to the processing at step S23 and appearing only once.

At the next step S25, the coloring processing section 42g refers to the boundary list and colors any boundary side in a boundary color (for example, red) that can be easily visually recognized by the user such as an operator. In this case, the thickness of a line drawing a boundary side may be increased (thickened) to allow easier visual recognition of the boundary side in color. In the boundary list illustrated in FIG. 16, the rightmost column indicates an enhancement color (boundary color) in which a boundary side is colored by the coloring processing section 42g. In the specific example illustrated in FIG. 16, R representing red is written as an enhancement color used in coloring. Alternatively, a boundary region within a distance equal to or smaller than a threshold from a boundary side may be colored in a boundary color or an enhancement color such as red.

Note that the processing of coloring a boundary side is not limited to execution at step S25, but may be performed in the processing at step S20 depending on whether the boundary enhancement is selected.

Note that, since the processing illustrated in FIG. 6 repeats similar processing in a loop as described above, when the boundary enhancement is selected and a region, an image of which is picked up by the image pickup section 25 changes with movement of the insertion section 11, a polygon list and a boundary list before the change are updated.

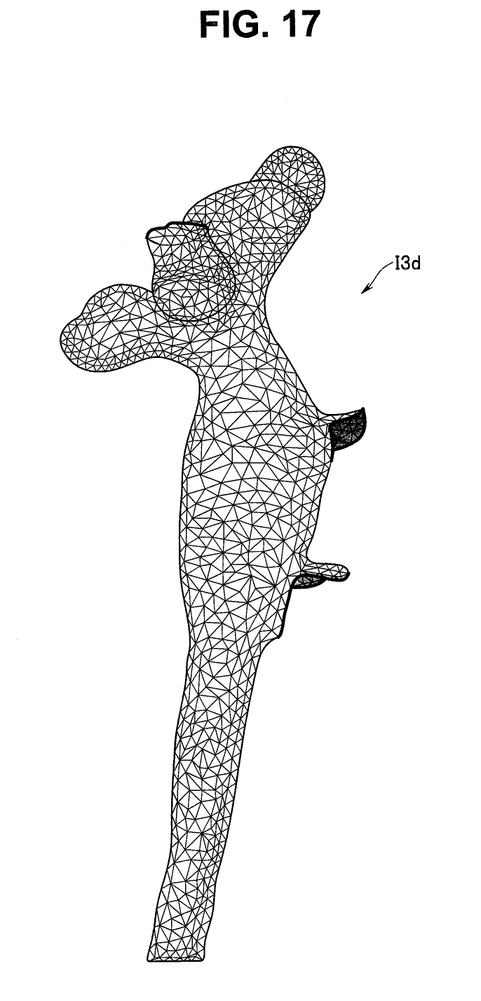

In this manner, when the boundary enhancement is selected, a 3D model image I3d corresponding to FIG. 13 is displayed on the monitor 8 as illustrated in FIG. 17.

In the 3D model image I3d illustrated in FIG. 17, a boundary side of each polygon in a boundary region in the 3D model image I3c illustrated in FIG. 13 is colored in an enhancement color. As illustrated in FIG. 17, a boundary side of each polygon in a structured region, which is positioned at a boundary with an unstructured region is colored in an enhancement color, and thus the user such as an operator can recognize the unstructured region adjacent to the boundary side in an easily visually recognizable state. Note that FIG. 17 is illustrated in monochrome display, and thus a boundary side illustrated with a line having a thickness larger than the thickness of an outline appears not largely different from the outline, but the boundary side is displayed in a distinct enhancement color. Thus, when the 3D model image I3d is displayed on the monitor 8 that performs color display, the boundary side can be visually recognized in a state largely different from the state of the outline. In the monochrome display, to facilitate identification of the boundary side from the outline, the boundary side may be displayed with a line having a thickness larger than the thickness of the outline by a threshold or more or a line having a thickness several times larger than the thickness of a line of the outline.

In this manner, the endoscope system and the image processing method according to the present embodiment can generate a three-dimensional model image in which an unstructured region is displayed in an easily visually recognizable manner.

In the present embodiment, since the 3D model image I3d in which the boundary between a structured region and an unstructured region is displayed in an enhanced manner is generated when the enhanced display is selected, the user such as an operator can recognize the unstructured region in a more easily visually recognizable state.

The following describes a first modification of the first embodiment. The present modification has a configuration substantially same as the configuration of the first embodiment, but in processing when the enhanced display is selected, a plane including a boundary side is enhanced instead of the boundary side as in the first embodiment.

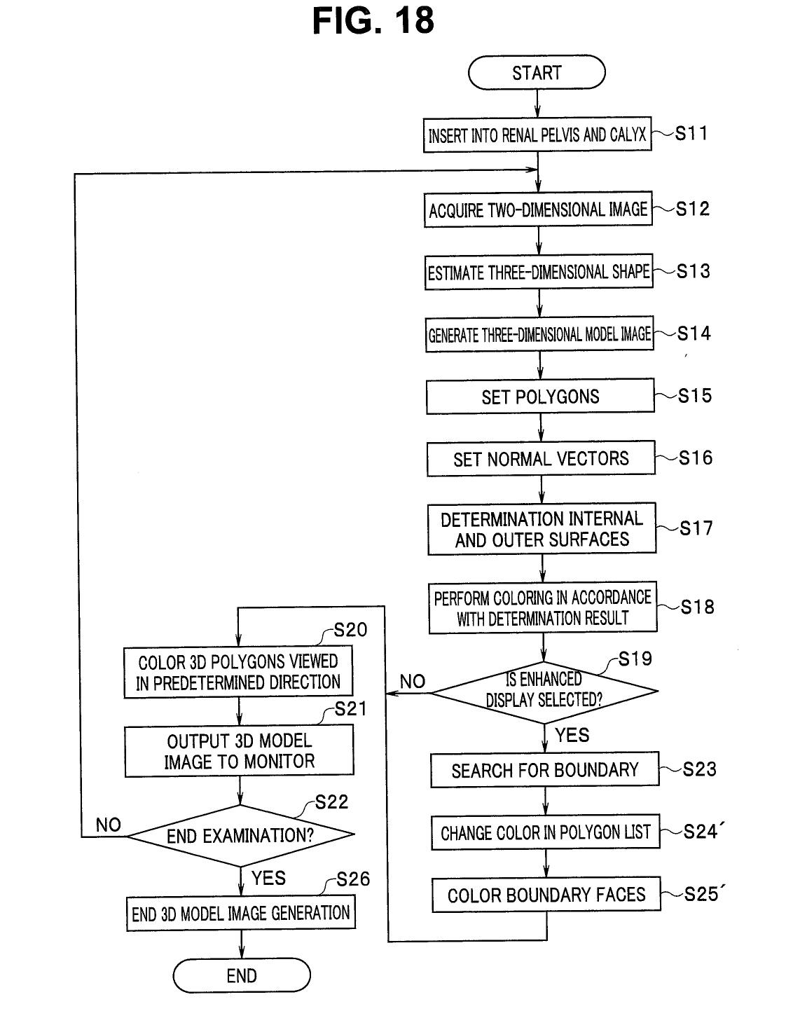

FIG. 18 illustrates the contents of processing in the present modification. In FIG. 18, the processing of producing (changing) a boundary list at step S24 in FIG. 6 is replaced with processing of changing a color of a polygon list, which is described at step S24′, and the processing of coloring a boundary side at step S25 is replaced with processing of coloring a boundary plane at step S25′. Any processing part different from processing in the first embodiment will be described below.

When the enhanced display is selected at step S19, the processing of searching for a boundary is performed at step S23, similarly to the first embodiment. In the processing at step S23, a polygon list as illustrated in FIG. 15 is produced, and a polygon having a boundary side as illustrated in FIG. 16 is extracted.

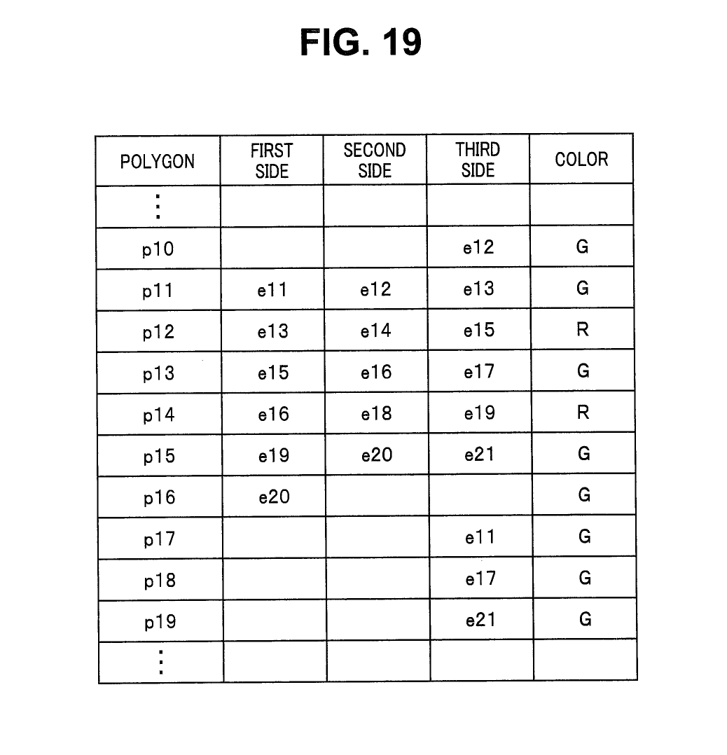

At the next step S24′, the boundary enhancement processing section 42f changes a color in the polygon list including a boundary side to an easily visually recognizable color (enhancement color) as illustrated in, for example, FIG. 19.

In the polygon list illustrated in FIG. 19, the colors of the polygons p12 and p14 including the respective boundary sides e14 and e18 in the polygon list illustrated in FIG. 15 are changed from gray to red.

In simple words, the enhancement color in FIG. 16 is a color for enhancing a boundary side, but in the present modification, the enhancement color is set to a color for enhancing the plane of a polygon including the boundary side. Note that, in this case, the plane may include the boundary side in the enhancement color.

At the next step S25′, the boundary enhancement processing section 42f colors, in the enhancement color, the plane of the polygon changed to the enhancement color, and then the process proceeds to the processing at step S20.

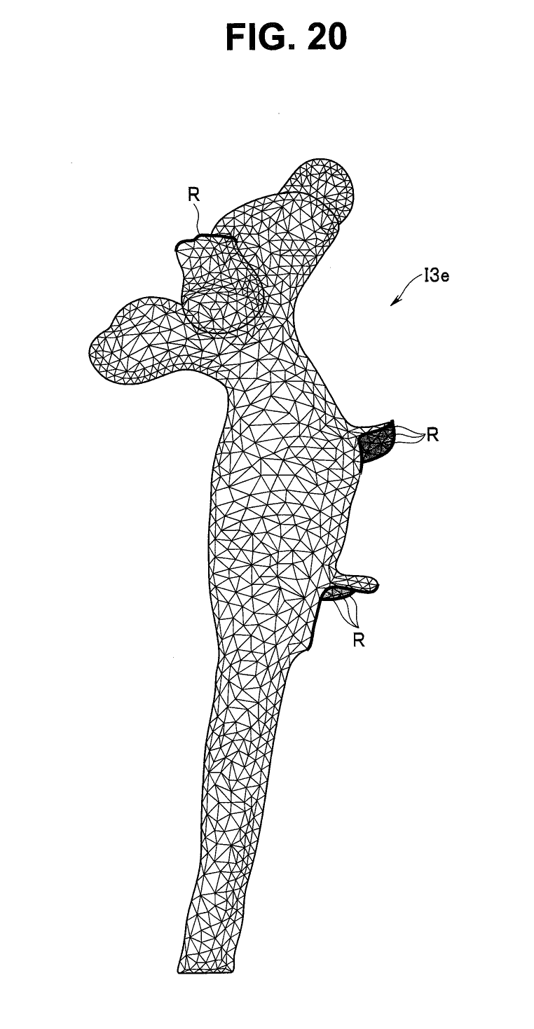

FIG. 20 illustrates a 3D model image I3e generated by the present modification and displayed on the monitor 8. In FIG. 20, the color of a polygon (that is, a boundary polygon) including a side adjacent to a boundary is an enhancement color (specifically, red R in FIG. 20). FIG. 20 illustrates an example in which a boundary side is also displayed in red in an enhanced manner.

The present modification achieves effects substantially same as the effects of the first embodiment. More specifically, when the enhanced display is not selected, effects same as the effects of the first embodiment when the enhanced display is not selected are achieved, and when the enhanced display is selected, a boundary plane including a boundary side of a boundary polygon is displayed in an easily visually recognizable enhancement color, and thus the effect of allowing the operator to easily recognize an unobserved region at a boundary of an observation region is achieved.

The following describes a second modification of the first embodiment. The present modification has a configuration substantially same as the configuration of the first embodiment, but in processing when the enhanced display is selected, processing different from processing in the first embodiment is performed. In the present modification, the boundary enhancement processing section 42f in the image generation section 42b in FIG. 2 is replaced with an enhancement processing section (denoted by 42f) corresponding to selection of the enhanced display (processing result is similar to a result obtained by the boundary enhancement processing section 42f).

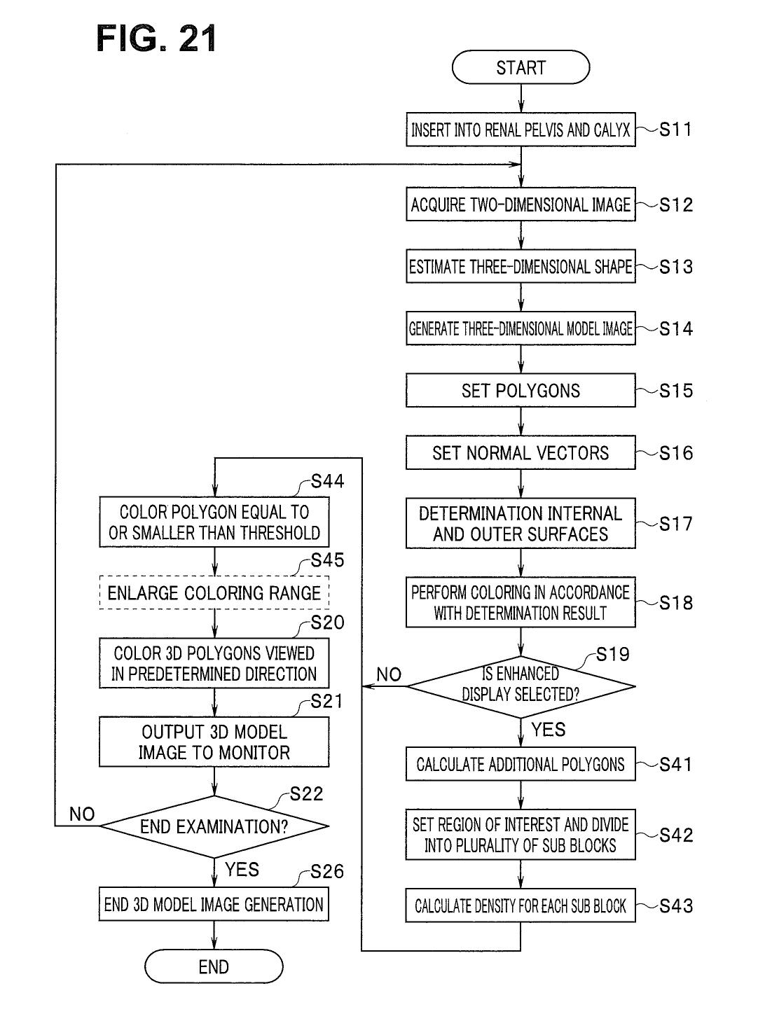

FIG. 21 illustrates processing in the present modification. In FIG. 21, when the enhanced display is not selected, processing same as processing in the first embodiment is performed. When the enhanced display is selected, the enhancement processing section 42f calculates any currently added polygon from a polygon list set after three-dimensional shape estimation in the last repetition as described at step S41.

Note that the addition is made to a polygon list in a blank state in the first processing, and thus the calculation is made on all polygons.

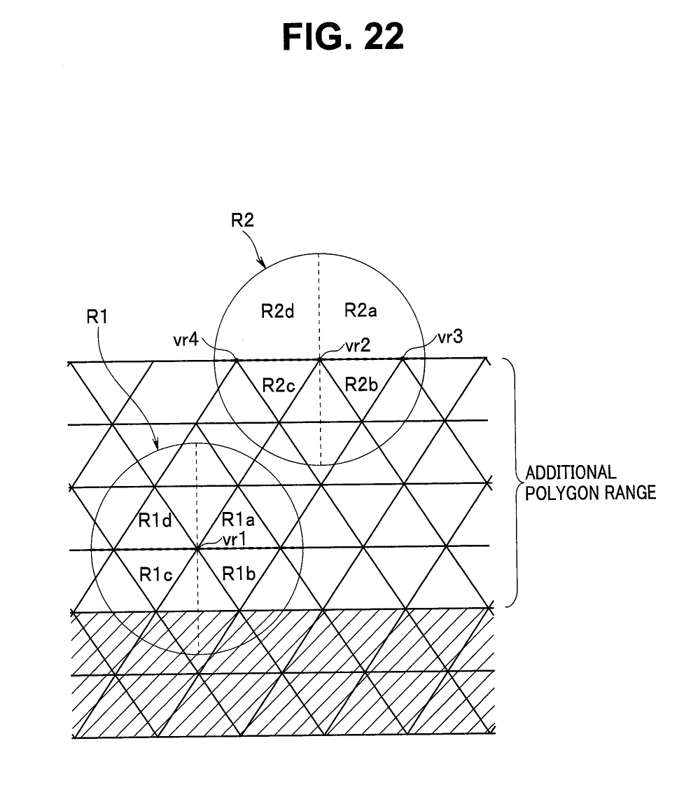

FIG. 22 illustrates the range of additional polygons acquired in the second processing for (the range of) hatched polygons acquired in the first processing. At the next step S42, the enhancement processing section 42f sets an interest region and divides polygons into a plurality of sub blocks.

As illustrated in FIG. 22, the enhancement processing section 42f sets, for example, a circular interest region centered at an apex (or the barycenter) of a polygon in the range of additional polygons and divides the interest region into, for example, four equally divided sub blocks as illustrated with dotted lines. In reality, for example, a spherical interest region is set to a three-dimensional polygon plane, and division into a plurality of sub blocks is performed.

FIG. 22 illustrates a situation in which interest regions R1 and R2 are set to respective apexes vr1 and vr2 of interest, the interest region R1 is divided into four sub blocks R1a, R1b, R1c, and R1d, and the interest region R2 is divided into four sub blocks R2a, R2b, R2c, and R2d.

At the next step S43, the enhancement processing section 42f calculates the density or number of apexes (or the barycenters) of polygons in each sub block. The enhancement processing section 42f also calculates whether the density or number of apexes (or the barycenters) of polygons has imbalance between sub blocks.

In the interest region R1, each sub block includes a plurality of apexes of continuously formed polygons and the like, and the density or number of apexes has small imbalance between sub blocks, whereas in the interest region R2, the density or number of apexes has large imbalance between the sub blocks R2b and R2c and between the sub blocks R2a and R2d. The sub blocks R2b and R2c have values substantially same as the value of the sub block R1a or the like in the interest region R1, but the sub blocks R2a and R2d do not include apexes (or the barycenters) of polygons except at the boundary, and thus have values smaller than the values of the sub blocks R2b and R2c. The number of apexes has large imbalance between the sub blocks R2b and R2c and between the sub blocks R2a and R2d.

At the next step S43, the enhancement processing section 42f performs processing of coloring a polygon satisfying a condition that the density or number of apexes (or the barycenters) of polygons has imbalance (equal to or larger than an imbalance threshold) between sub blocks and the density or number of apexes (or the barycenters) of polygons is equal to or smaller than a threshold, or apexes of the polygon in an easily visually recognizable color (enhancement color such as red). In FIG. 22, for example, the apexes vr2, vr3, and vr4 or polygons sharing the apexes are colored. After the processing at step S44 or after step S45 is performed, the process proceeds to the processing at step S20.

When coloring is performed in this manner, the user can perform, through the enhanced display selection section 44b of the input apparatus 44, selection for increasing a coloring range to obtain visibility for achieving easier visual recognition. When the selection for increasing the coloring range is performed, processing of increasing the coloring range is performed as described below.

For the processing S44 of coloring a polygon satisfying the above-described condition (referred to as a first condition) that imbalance exists in density or the like or any apex of the polygon, the enhancement processing section 42f further enlarges the coloring range at step S45 illustrated with a dotted line in FIG. 21. As described above, the processing at step S45 illustrated with a dotted line is performed when selected.

The enhancement processing section 42f colors (any apex of) a polygon satisfying the first condition as described at step S44, but also colors (any apex of) a polygon positioned within a constant distance from (any apex of) a polygon matching the first condition at step S45 and added simultaneously with (the apex of) the polygon matching the first condition.

In such a case, for example, the first uppermost polygons in the horizontal direction or the first and second uppermost polygons in the horizontal direction in FIG. 22 are colored. The range of polygons to be colored can be increased by increasing the constant distance.

Note that it can be regarded that newly added points (vr2, vr3, and vr4 in FIG. 22) around which a boundary exists satisfy a second condition for coloring in an easily visually recognizable color.

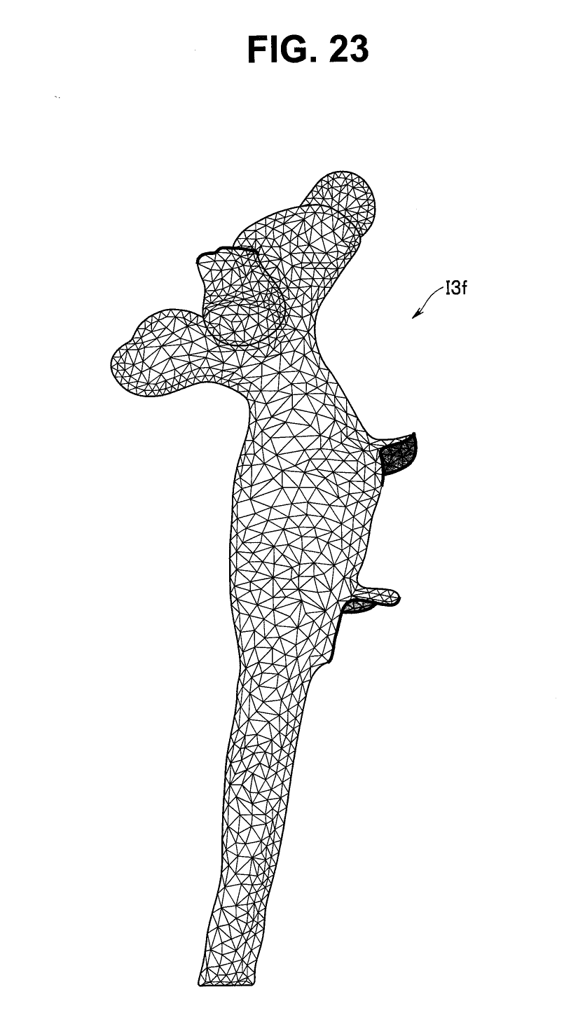

FIG. 23 illustrates exemplary display of a 3D model image I3f according to the present modification. The 3D model image I3f is substantially same as the 3D model image I3e illustrated in FIG. 20. Note that FIG. 23 omits notation of coloring of a polygon or the like adjacent to a boundary in FIG. 20 in R as an enhancement color. The present modification achieves effects substantially same as the effects of the first embodiment. That is, when the enhanced display is not selected, effects same as the effects of the first embodiment when the enhanced display is not selected are achieved, and when the enhanced display is selected, a boundary region of any structured polygon can be displayed distinct in an easily visually recognizable color, similarly to the first embodiment when the enhanced display is selected. Thus, any unstructured region positioned adjacent to the boundary region and yet to be observed can be easily recognized.

The following describes a third modification of the first embodiment.

The present modification corresponds to a case in which display similar to display when the enhanced display is selected is performed even when the enhanced display is not selected in the first embodiment.

Accordingly, the present modification corresponds to a configuration in which the input apparatus 44 does not include the enhanced display selection section 44b in the configuration illustrated in FIG. 2, and the boundary enhancement processing section 42f does not need to be provided, but processing similar to processing performed by the boundary enhancement processing section 42f is performed effectively. The other configuration is substantially same as the configuration of the first embodiment.

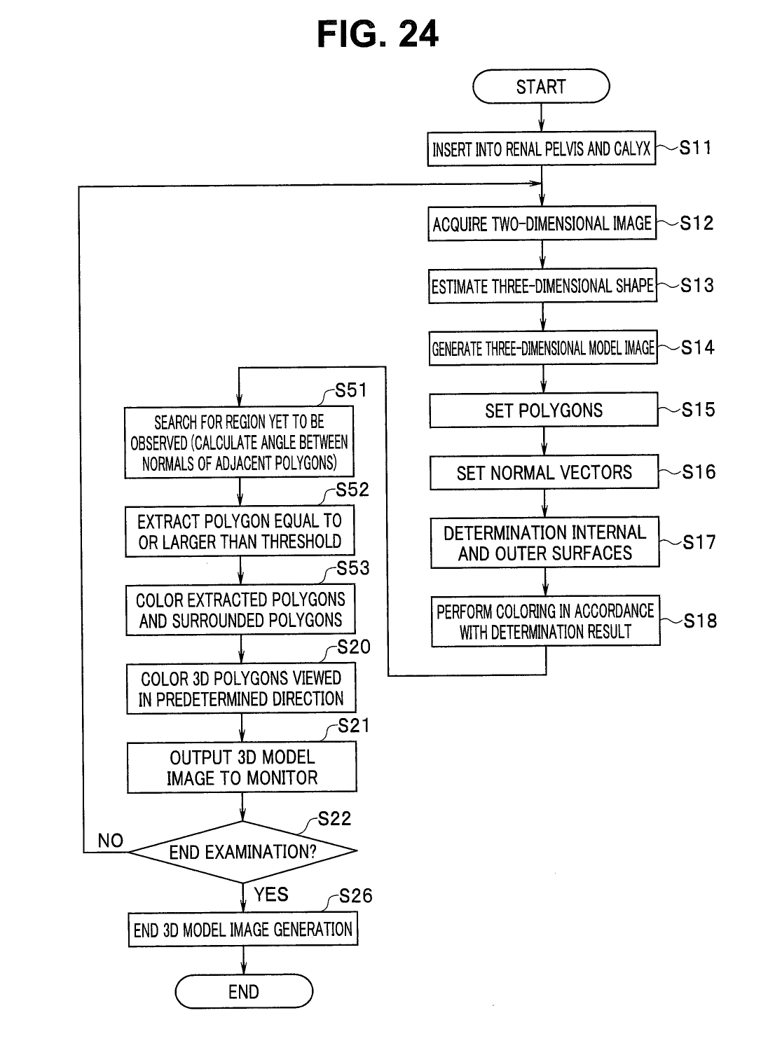

FIG. 24 illustrates the contents of processing in the present modification. The flowchart in FIG. 24 illustrates processing similar to the flowchart illustrated in FIG. 6, and thus the following description will be made only on any different part of the processing.

Steps S1 to S18 are processing same as the corresponding processing in FIG. 6, and after the processing at step S18, the polygon processing section 42c performs the processing of searching for an unobserved region at step S51.

As described above, the three-dimensional shape estimation is performed at step S13 and the processing of generating a 3D model image is performed through processing of bonding polygons to the surface of an observed region, but when an unobserved region exists as an opening portion in, for example, a circle shape (adjacent to the observed region) at a boundary of the observed region, processing performed on a plane in the observed region is potentially performed on the opening portion by bonding polygons to the opening portion.

Thus, in the present modification, in the processing of searching for an unobserved region at step S51, an angle between the normal of a polygon set to a region of interest and the normal of a polygon positioned adjacent to the polygon and set in the observed region is calculated, and whether the angle is equal to or larger than a threshold of approximately 90° is determined.

At the next step S52, the polygon processing section 42c extracts polygons, the angle between the two normals of which is equal to or larger than the threshold.

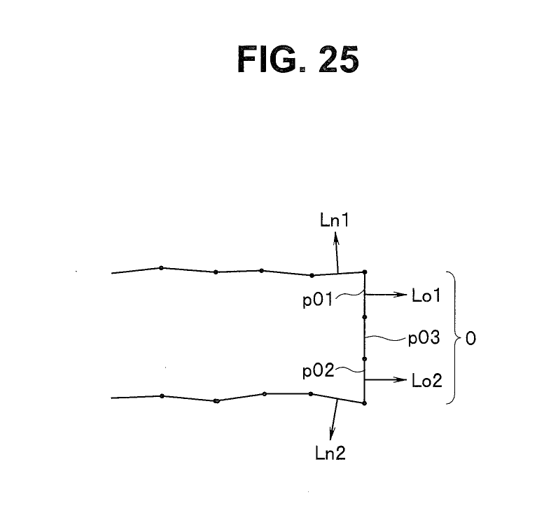

FIG. 25 illustrates an explanatory diagram of an operation in the present modification. FIG. 25 illustrates an exemplary situation in which polygons are set at an observed luminal shape part extending in the horizontal direction, and a substantially circular opening portion O as an unobserved region exists at the right end of the part.

In this case, similarly to a case in which polygons are set in the observed region adjacent to the opening portion O, processing of setting polygons to the opening portion O is potentially performed. In such a case, the angle between a normal Ln1 of a polygon set in the observed region adjacent to a boundary of the opening portion O and a normal Lo1 of a polygon pO1 positioned adjacent to the polygon and set to block the opening portion O is significantly larger than the angle between two normals Lni and Lni+1 set to two polygons adjacent to each other in the observed region, and is equal to or larger than a threshold.

FIG. 25 illustrates, in addition to the normals Ln1 and Lo1, a normal Ln2 and a normal Lo2 of a polygon pO2 set to block the opening portion O.

At the next step S53, the coloring processing section 42g colors, in a color (for example, red) different from a color for the observed region, a plurality of polygons (polygons pO1 and pO2 in FIG. 25), the angle between the two normals of which is equal to or larger than the threshold, and a polygon (polygon pO3 between the polygons pO1 and pO2) surrounded by a plurality of polygons. After the processing at step S53, the process proceeds to the processing at step S20.

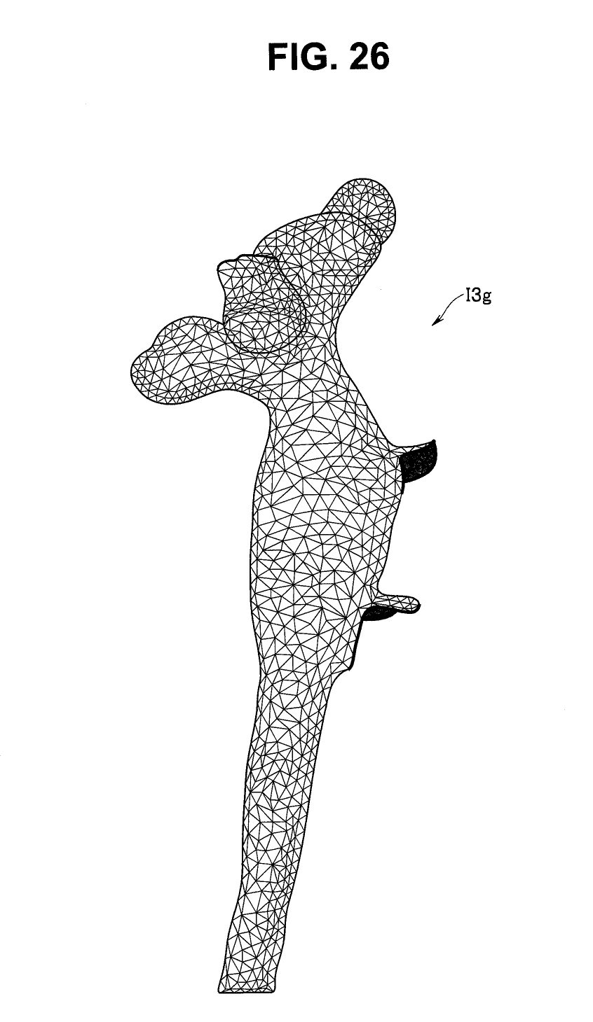

FIG. 26 illustrates a 3D model image I3g according to the present modification. In FIG. 26, an unobserved region is displayed in red.

According to the present modification, when a polygon is set adjacent to a polygon in an observed region and set in an unobserved region, the polygon can be colored to facilitate visual recognition of the unobserved region.

The following describes a fourth modification of the first embodiment.

The present modification allows easy recognition of an unobserved region by simplifying the shape of the boundary between an observed region and the unobserved region (to reduce the risk of false recognition that, for example, a complicated shape is attributable to noise).

In the present modification, in the configuration illustrated in FIG. 2, the input apparatus 44 includes a smoothing selection section (denoted by 44c) for selecting smoothing in place of the enhanced display selection section 44b, and the image generation section 42b includes a smoothing processing section (denoted by 42h) configured to perform smoothing processing in place of the boundary enhancement processing section 42f. The other configuration is substantially same as the configuration of the first embodiment.

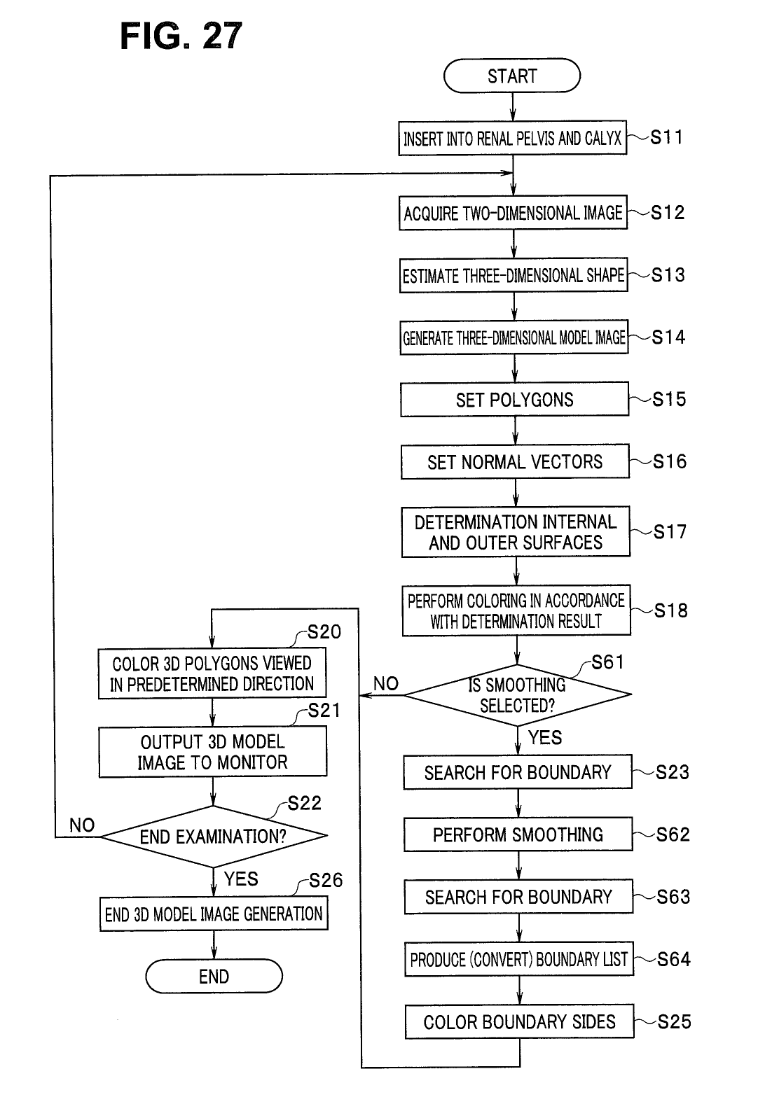

FIG. 27 illustrates the contents of processing in the present modification. The processing illustrated in FIG. 27 is similar to the processing illustrated in FIG. 6, and thus the following description will be made only on any different part of the processing.

In the processing illustrated in FIG. 27, the processing at step S19 in FIG. 6 is replaced with processing of determining whether to select smoothing at step S61.

Smoothing processing at step S62 is performed after the boundary search processing at step S23, and boundary search processing is further performed at step S63 after the smoothing processing, thereby producing (updating) a boundary list.

In the present modification, to display the shape of the boundary between an observed region and an unobserved region in a simplified manner as described above, a polygon list before the smoothing processing at step S62 is performed is held in, for example, the information storage section 43, and a held copy is set to a polygon list and used to generate a 3D model image (the copied polygon list is changed by smoothing, but the polygon list before the change is held in the information storage section 43).

In the processing at step S61 in FIG. 27, when smoothing is not selected, the process proceeds to step S20 where the processing described in the first embodiment is performed.

When smoothing is selected, the polygon processing section 42c performs the processing of searching for a boundary at step S23.

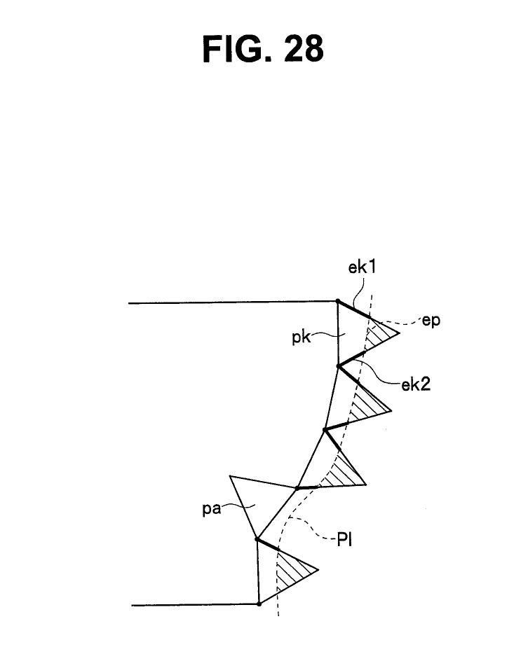

The processing of searching for a boundary at step S23 is described with reference to, for example, FIGS. 14 to 16. Through the processing of searching for a boundary, a polygon boundary is extracted as illustrated in, for example, FIG. 28 in some cases. FIG. 28 schematically illustrates a situation in which a polygon boundary part of the luminal shape illustrated in FIG. 25 has a complicated shape including uneven portions.

At the next step S62, the smoothing processing section 42h performs smoothing processing. The smoothing processing section 42h applies, for example, a least-square method to calculate a curved surface Pl (the amount of change in the curvature of which is restricted in an appropriate range), the distances of which from the barycenters (or apexes) of a plurality of polygons in a boundary region are minimized. When the degree of unevenness between adjacent polygons is large, the present invention is not limited to application of the least-square method to all polygons adjacent to the boundary, but the least-square method may be applied only to some of the polygons.

In addition, the smoothing processing section 42h performs processing of deleting any polygon part outside of the curved surface Pl. In FIG. 28, deleted polygon parts are hatched.

At the next step S63, the smoothing processing section 42h (or the polygon processing section 42c) searches for a polygon forming a boundary region in processing corresponding to the above-described processing (steps S23, S62, and S63). For example, processing of searching for a polygon (for example, a polygon pk denoted by a reference sign) partially deleted by the curved surface Pl, and for a polygon pa, a side of which is adjacent to the boundary as illustrated in FIG. 28 is performed.

Then, at the next step S64, a boundary list in which sides of the polygons extracted through the search processing are set as boundary sides is produced (updated). In this case, an apex is newly added to a polygon partially deleted by the curved surface Pl so that the shape of the polygon becomes a triangle, and then the polygon is divided. Note that boundary sides of the polygon pk in FIG. 28 are sides ek1 and ek2 partially deleted by the curved surface Pl and a side ep as the curved surface Pl. In this case, the side ep as the curved surface Pl is approximated with a straight side connecting both ends in the plane of the polygon pk.

At the next step S25, the coloring processing section 42g performs processing of coloring, in an easily visually recognizable color, the boundary sides of polygons written in the boundary list, and thereafter, the process proceeds to the processing at step S20.

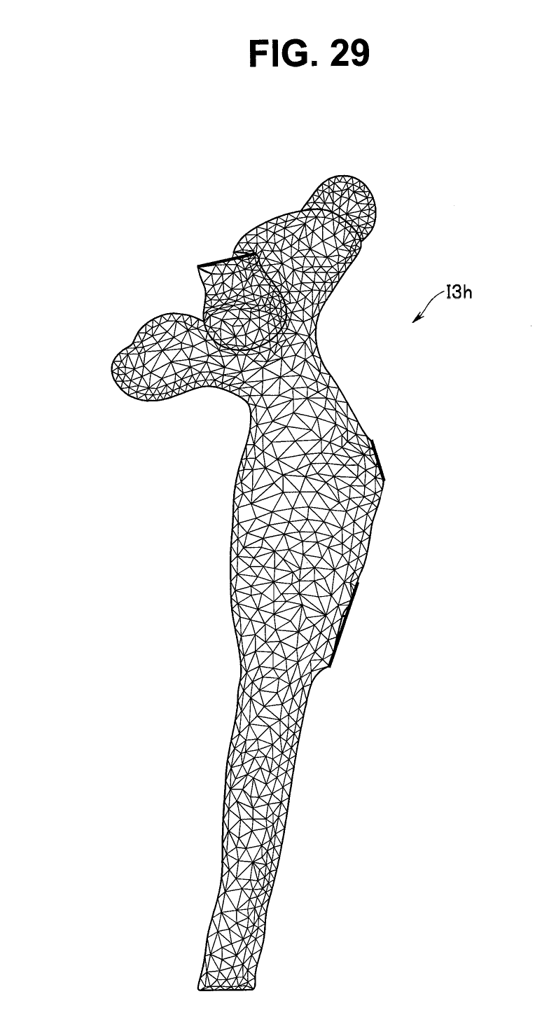

FIG. 29 illustrates a 3D model image I3h generated in this manner and displayed on the monitor 8. According to the present modification, any boundary part having a complicated shape is displayed as a simplified boundary side in an easily visually recognizable color, thereby facilitating recognition of an unobserved region.

Note that processing may be performed by the following method instead of the polygon division by the curved surface Pl.

At step S62, the smoothing processing section 42h searches for an apex outside of the curved surface Pl. At the next step S63, the smoothing processing section 42h (or the polygon processing section 42c) performs processing of deleting a polygon including an apex outside of the curved surface Pl from a copied polygon list. At the next step S63, in processing corresponding to the above-described processing (steps S23, S62, and S63), the smoothing processing section 42h (or the polygon processing section 42c) performs the processing of deleting a polygon including an apex outside of the curved surface Pl from the copied polygon list, and performs the boundary search described in another modification.

The following describes a fifth modification of the first embodiment.

In the first embodiment, when the enhanced display is selected, the processing of extracting a side of a polygon in a boundary region as a boundary side and coloring the boundary side in a visually recognizable manner is performed, but in the present modification, when a three-dimensional shape is expressed with points (corresponding to, for example, points at the barycenters of polygons or apexes of the polygons) instead of the polygons, processing of extracting, as boundary points, points at a boundary in place of boundary sides (of the polygons) is performed, and processing of coloring the boundary points in an easily visually recognizable manner is performed.

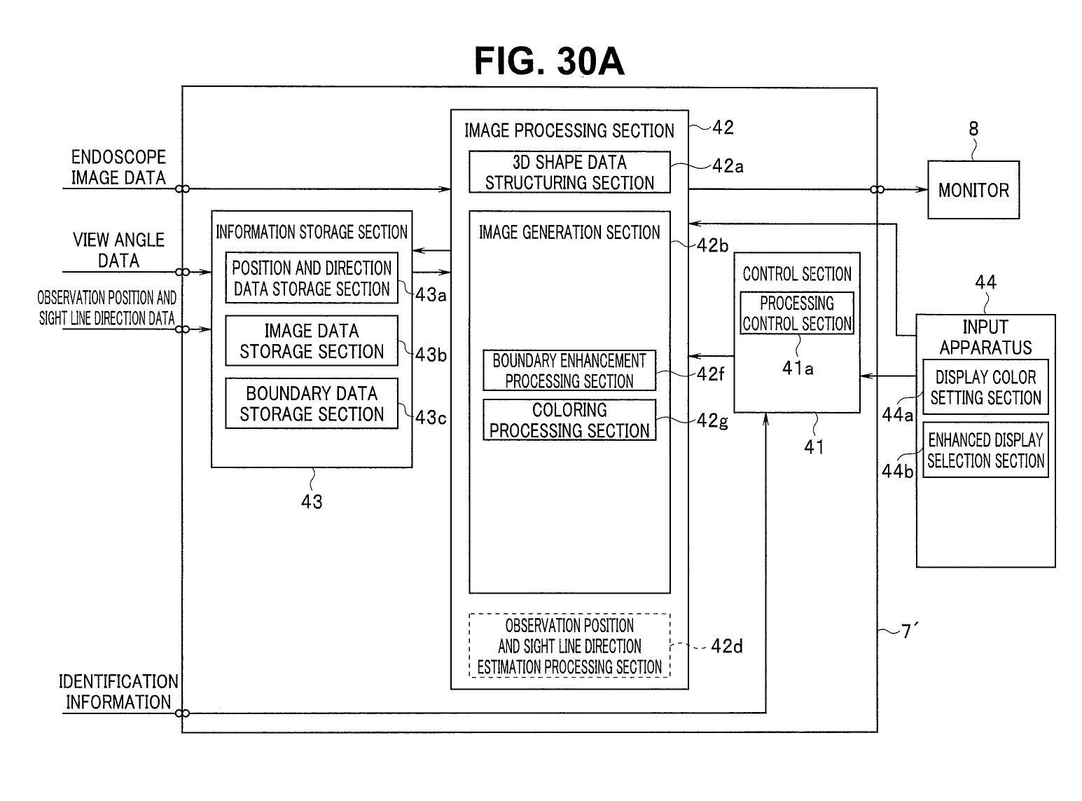

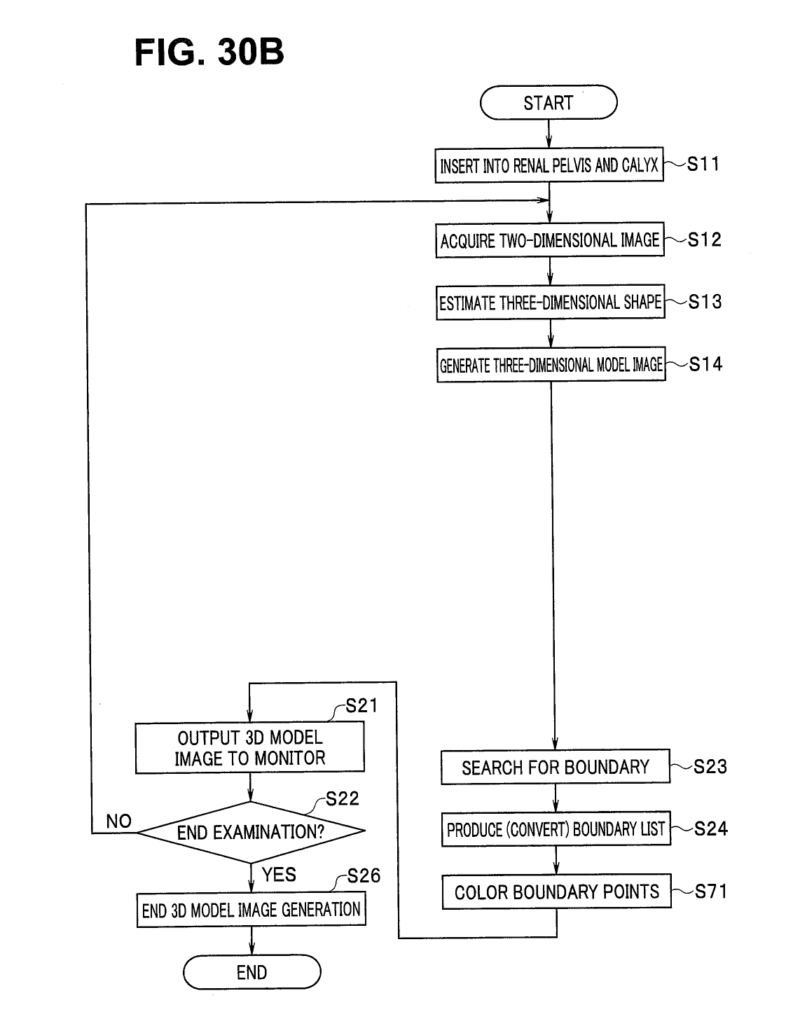

Thus, in the present modification, the boundary enhancement processing section 42f performs processing of enhancing a boundary point in the configuration illustrated in FIG. 2. FIG. 30A illustrates the configuration of an image processing apparatus 7′ in the present modification. The image processing apparatus 7′ in the present modification does not perform, for example, processing of displaying a three-dimensional shape with polygons, and thus does not include the polygon processing section 42c and the inner and outer surface determination section 42e illustrated in FIG. 2. The other configuration is substantially same as the configuration of the first embodiment.

FIG. 30B illustrates the contents of processing in the present modification. The flowchart in FIG. 30B illustrates processing similar to the flowchart illustrated in FIG. 6, and thus the following description will be made only on any different part of the processing. In the flowchart illustrated in FIG. 30B, the processing at steps S15 to S20 in FIG. 6 is not performed. Thus, the process proceeds to the processing at steps S23 and S24 after the processing at step S14, performs processing of coloring a boundary point as described at step S71 in place of the processing of coloring a boundary side at step S25 in FIG. 6, and proceeds to the processing at step S21 after the processing at step S71. However, as described below, for example, the contents of the processing of producing (changing) a boundary list at step S24 same as step S24 in FIG. 6 is slightly different from the contents of processing in the first embodiment.

At step S23, in processing of searching for a boundary and extracting a boundary point, the boundary enhancement processing section 42f may extract a boundary point through the processing (processing of satisfying at least one of the first condition and the second condition) described with reference to FIG. 22 in the second modification.

That is, as for the first condition, a plurality of interest regions are set to a point (barycenter or apex) of interest, the density of points or the like in a sub block of each interest region is calculated, and any point satisfying a condition that the density or the like has imbalance and the density has a value equal to or smaller than a threshold is extracted as a boundary point.

Alternatively, as for the second condition, a newly added point around which a boundary exists is extracted as a boundary point. In the example illustrated in FIG. 22, vr2, vr3, vr4, and the like are extracted as boundary points.

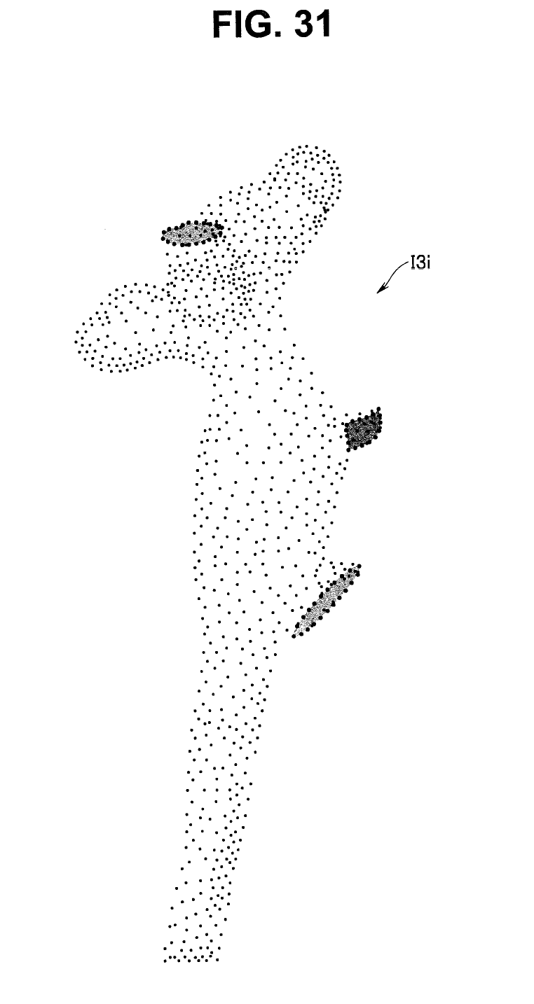

FIG. 31 illustrates a 3D model image I3i generated by the present modification and displayed on the monitor 8. As illustrated in FIG. 31, points in boundary regions are displayed in an easily visually recognizable color. Note that a point in a boundary region may be colored as a bold point (having an increased area) in an easily visually recognizable color (enhancement color). In addition, a middle point between two adjacent points in a boundary region may be displayed in an easily visually recognizable color.

According to the present modification, a point at the boundary between an observed structured region and an unobserved unstructured region is displayed in an easily visually recognizable color, and thus the unstructured region can be easily recognized. Note that a line (referred to as a border line) connecting the above-described adjacent boundary points may be drawn and colored in an easily visually recognizable color by the coloring processing section 42g. In addition, any point included within a distance equal to or smaller than a threshold from a boundary point may be colored as a bold point (having an increased area) in an easily visually recognizable color (enhancement color).

Note that a three-dimensional shape can be displayed with the barycenters of observed polygons in the present modification. In this case, processing of calculating the barycenters of polygons is performed. The processing may be applied to a sixth modification describes below.

In the processing at step S71 in FIG. 30B according to the fifth modification, any surrounding point near a boundary point may be additionally colored in an easily visually recognizable color together with the boundary point (refer to FIG. 33). The sixth modification of the first embodiment in which a processing result substantially same as a processing result in this case will be described next.

In the sixth modification, a boundary point and any surrounding point around the boundary point in the fifth modification are colored and enhanced in an easily visually recognizable color, and a configuration same as the configuration according to the fifth modification is employed.

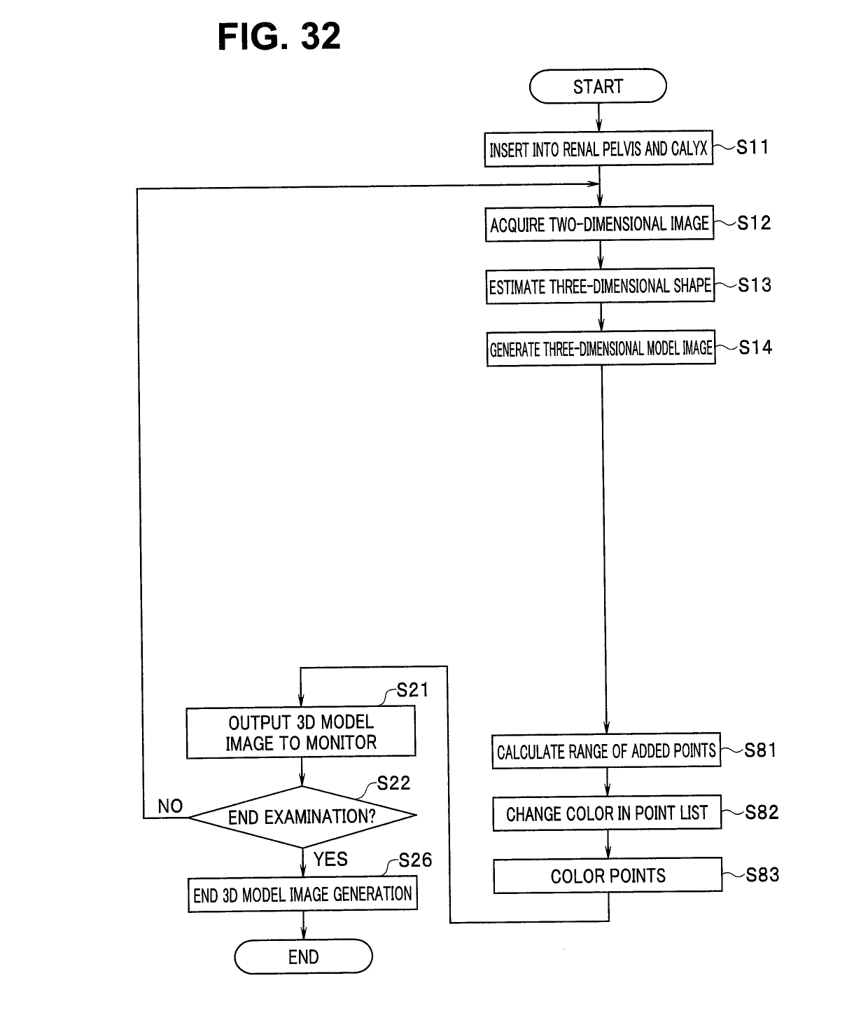

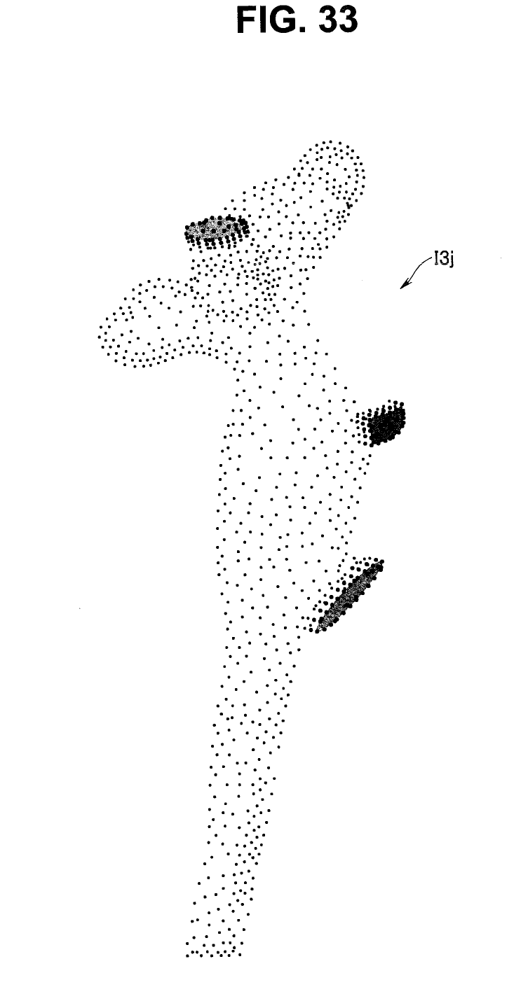

FIG. 32 illustrates the contents of processing in the present modification. The processing illustrated in FIG. 32 is similar to the processing according to the fifth modification of the first embodiment illustrated in FIG. 30B, and processing at steps S81 to S83 is performed after the processing at step S14, and the process proceeds to the processing at step S21 after the processing at step S83. After the processing at step S14, the boundary enhancement processing section 42f performs processing of calculating any added point since the last repetition as described at step S81.

The example of range of added points is same as, for example, the range in the case with polygons described with reference to FIG. 22. At the next step S82, the boundary enhancement processing section 42f changes the color of any newly added point in a point list that is a list of added points to a color (for example, red) different from an observed color. The boundary enhancement processing section 42f also performs processing of setting, back to the observed color, the color of a point in the different color at a distance equal to or larger than a threshold from a newly added point in the point list.

At the next step S83, the coloring processing section 42g performs processing of coloring points of polygons in accordance with colors written in the polygon list up to the previous step S82, and then the process proceeds to the processing at step S21.

FIG. 33 illustrates a 3D model image I3j according to the present modification. In addition to boundary points in the example illustrated in FIG. 31, points around the boundary points are colored and displayed in the same color, which makes it easier for the operator to check an unobserved region.

For example, only an unobserved region may be displayed in accordance with an operation of the input apparatus 44 by the user. When any observed region is not displayed, the operator can easily check an unobserved region behind the observed region. Note that the function of displaying only an unobserved region may be provided to any other embodiment or modification.

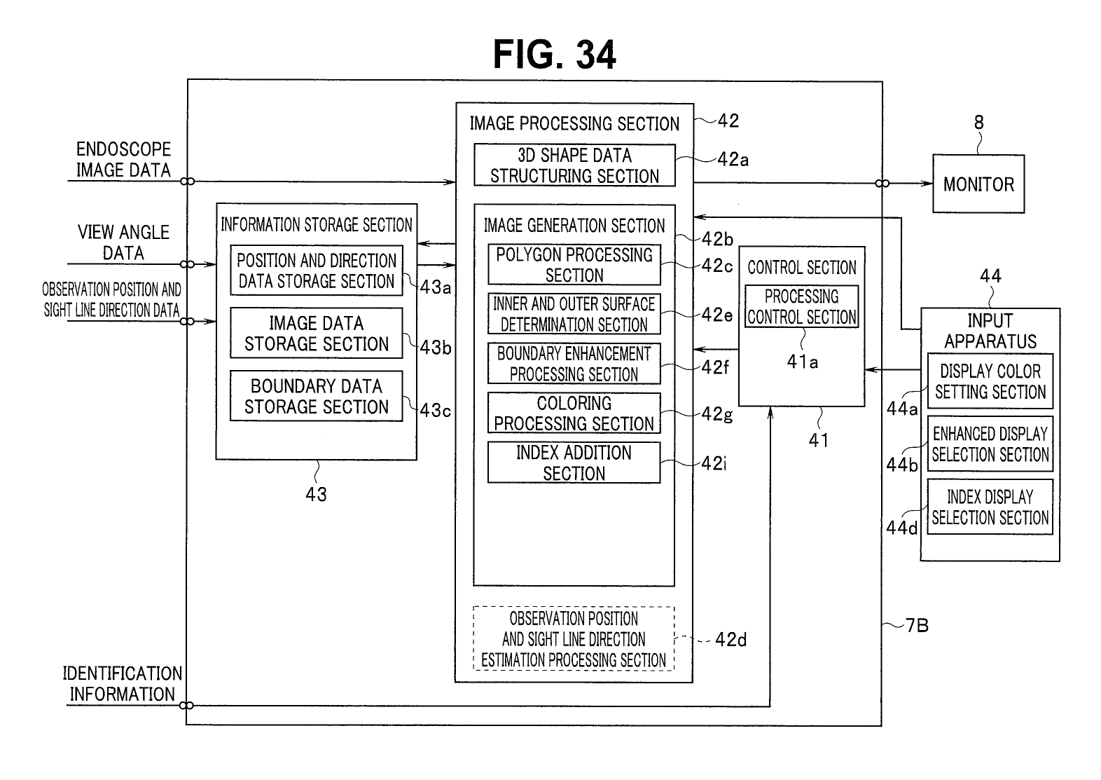

The following describes a seventh modification of the first embodiment. In the present modification, an index indicating an unobserved region is added and displayed, for example, when index addition is selected in the first embodiment. FIG. 34 illustrates the configuration of an image processing apparatus 7B in the present modification.

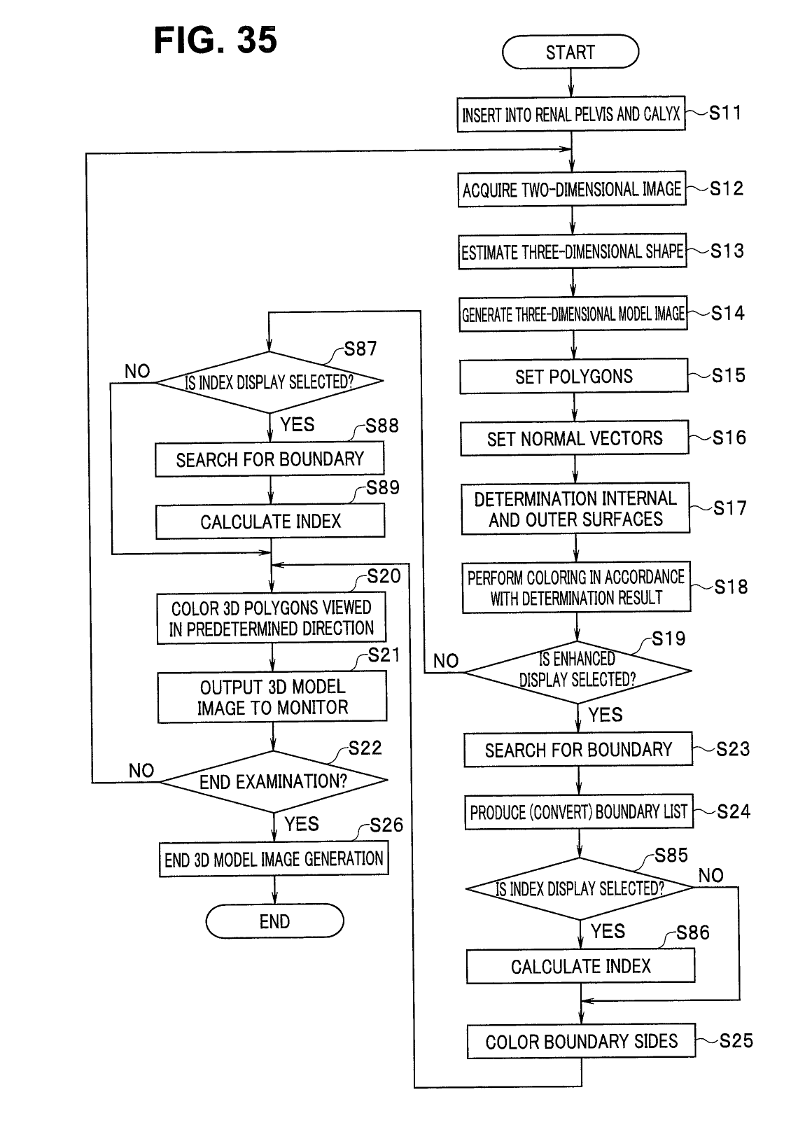

In the image processing apparatus 7B, the input apparatus 44 in the image processing apparatus 7 illustrated in FIG. 2 includes an index display selection section 44d for selecting index display, and the image generation section 42b in the image processing apparatus 7 illustrated in FIG. 2 includes an index addition section 42i configured to add an index to an unobserved region. The other configuration is same as the configuration of the first embodiment. FIG. 35 illustrates the contents of processing in the present modification.

The flowchart illustrated in FIG. 35 has contents of processing in which, in addition to the flowchart illustrated in FIG. 6, processing for displaying an index in accordance with a result of the index display selection is additionally performed.

When the enhanced display is selected at step S19, after the processing at steps S23 and S24 is performed, the control section 41 determines whether index display is selected at step S85. When index display is not selected, the process proceeds the processing at step S25, or when index display is selected, the index addition section 42i performs processing of calculating an index to be added and displayed at step S86, and then, the process proceeds to the processing at step S25.

The index addition section 42i

a. calculates a plane including a side at a boundary,
b. subsequently calculates the barycenter of a point at the boundary, and
c. subsequently calculates a point on a line parallel to the normal of the plane calculated at “a” and at a constant distance from the barycenter of the point at the boundary and adds an index.

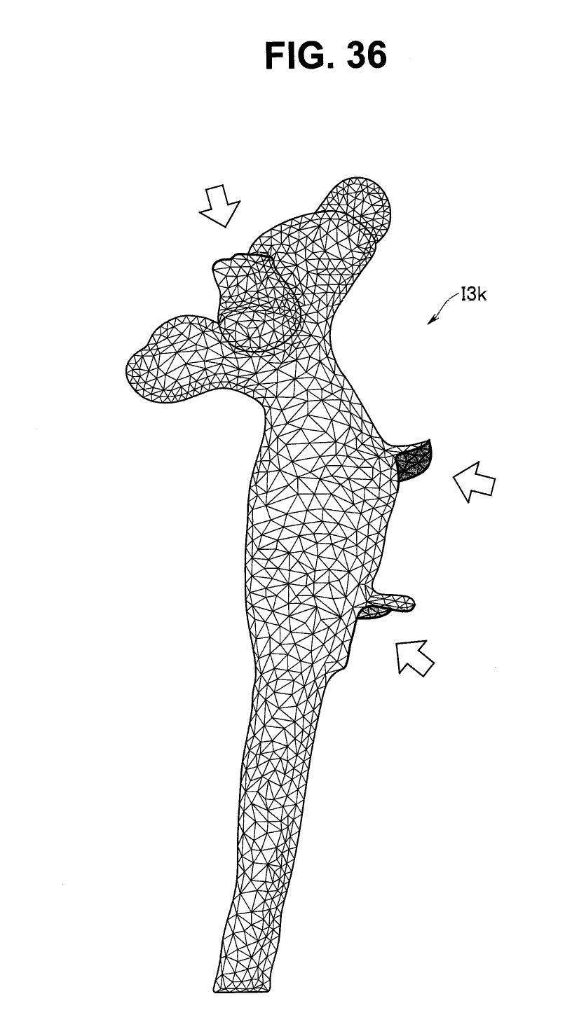

FIG. 36 illustrates a 3D model image I3k in this case. FIG. 36 is a diagram in which indexes are added to the 3D model image I3d illustrated in FIG. 17.

When the enhanced display is not selected at step S19 in FIG. 35, the control section 41 determines whether index display is selected at step S87. When index display is not selected, the process proceeds to the processing at step S20, or when index display is selected, similarly to step S23, the processing of searching for a boundary is performed at step S88, and then, the index addition section 42i performs processing of calculating an index to be added and displayed at step S89, before the process proceeds to the processing at step S20.

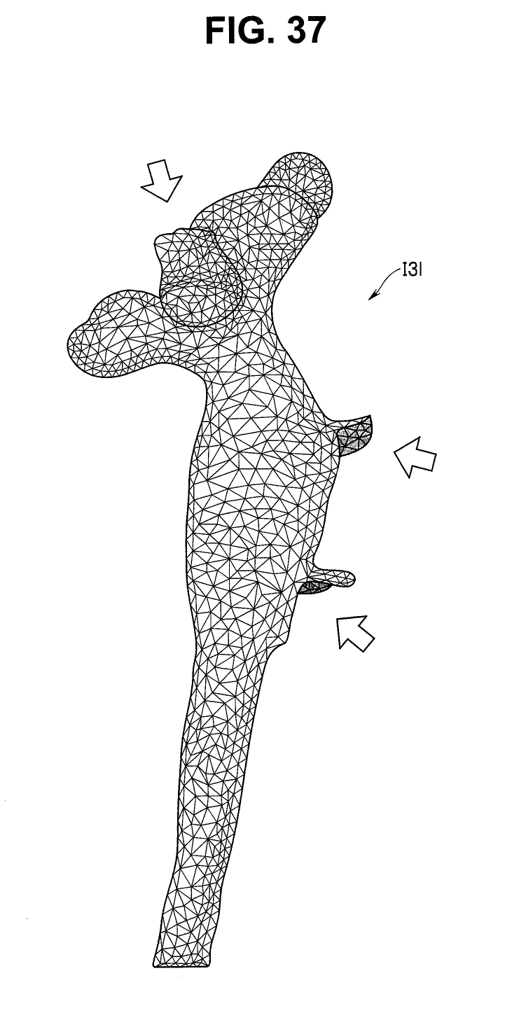

FIG. 37 illustrates a 3D model image I31 in this case. FIG. 37 is a diagram in which indexes are added to the 3D model image I3c illustrated in FIG. 13. Note that the indexes are colored in, for example, yellow.

According to the present modification, selection for displaying the 3D model images I3c and I3d as in the first embodiment can be performed, and also, selection for displaying the 3D model images I31 and I3k to which indexes are added can be performed. Indexes may be displayed on 3D model images I13e, I13f, I13g, I13h, I13i, and I13j by additionally performing the same processing.

The following describes an eighth modification of the first embodiment. The seventh modification describes the example in which an index illustrating a boundary or an unobserved region with an arrow is displayed outside of the 3D model images I3c and I3d. Alternatively, index display in which light from a light source set inside a lumen in a 3D model image leaks out of an opening portion as an unobserved region may be performed as described below.

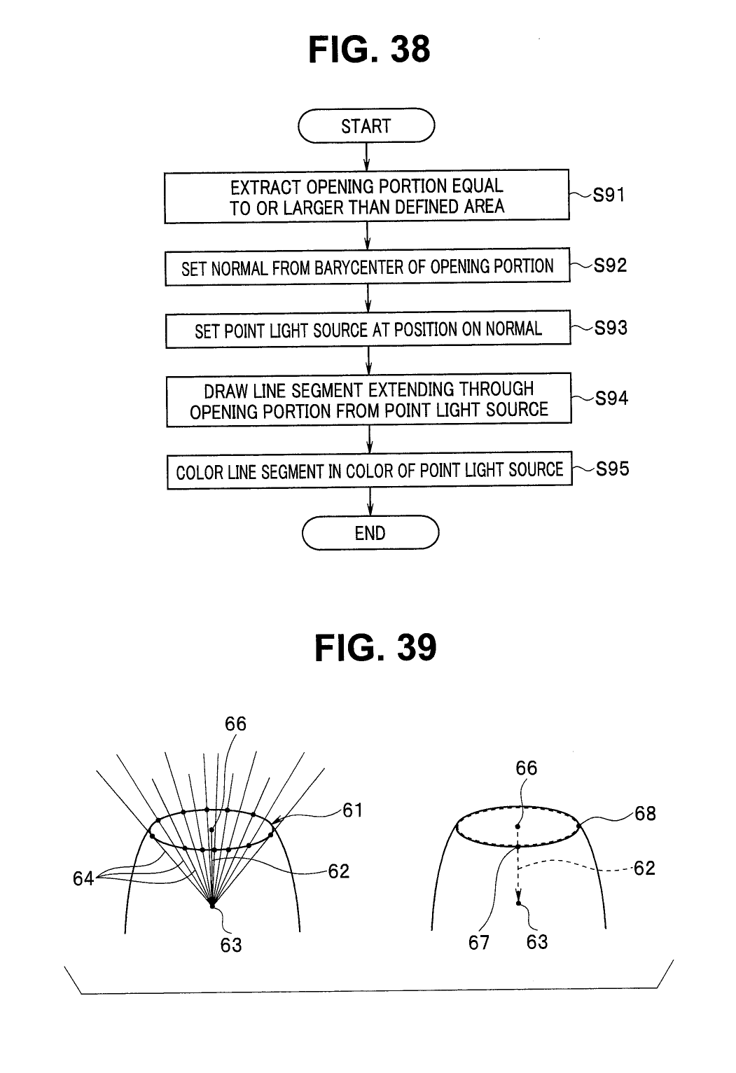

In processing according to the present modification, only the processing of calculating an index at step S86 or S89 in FIG. 36 in the seventh modification is replaced with processing of generating an index illustrated in FIG. 39. Note that the index addition section 42i functions as an opening portion extraction section configured to extract an opening portion as an unstructured region having an area equal to or larger than predetermined area when processing described below with reference to FIG. 38 or the like is performed, and a light source setting section configured to set a point light source at a position on a normal extending on the internal side of a lumen.

FIG. 38 illustrates the contents of processing of generating an index in the present modification.

When the processing of generating an index is started, the index addition section 42i calculates an opening portion as an unobserved region that has an area equal to or larger than a defined area at the first step S91. FIG. 39 illustrates an explanatory diagram for the processing illustrated in FIG. 38, and illustrates an opening portion 61 as an unobserved region that has an area equal to or larger than a defined area (or predetermined area) in a luminal organ.

At the next step S92, the index addition section 42i sets (on the internal side of the lumen) a normal 62 from the barycenter of points included in the opening portion 61. As illustrated in a diagram on the right side in FIG. 39, the normal 62 is a normal to a plane passing through a total of three points of a barycenter 66, a point 67 nearest to the barycenter 66, and a point 68 farthest from the barycenter 66 among the points included in the opening portion 61, and has a unit length from the barycenter 66. The direction of the normal is a direction in which a large number of polygons forming a 3D model exist. Note that three representative points set on the opening portion 61 as appropriate may be employed in place of the above-described three points.

At the next step S93, the index addition section 42i sets a point light source 63 at a defined length (inside the lumen) along the normal 62 from the barycenter 66 of the opening portion 61.

At the next step S94, the index addition section 42i draws line segments 64 extending from the point light source 63 toward the outside of the opening portion 61 through (respective points on) the opening portion 61.

At the next step S95, the index addition section 42i colors the line segments 64 in the color (for example, yellow) of the point light source 63. Display with added indexes may be performed by performing processing as described below in addition to the processing illustrated in FIG. 38. Steps S91 to S93 illustrated in FIG. 38 are same in the processing described below.

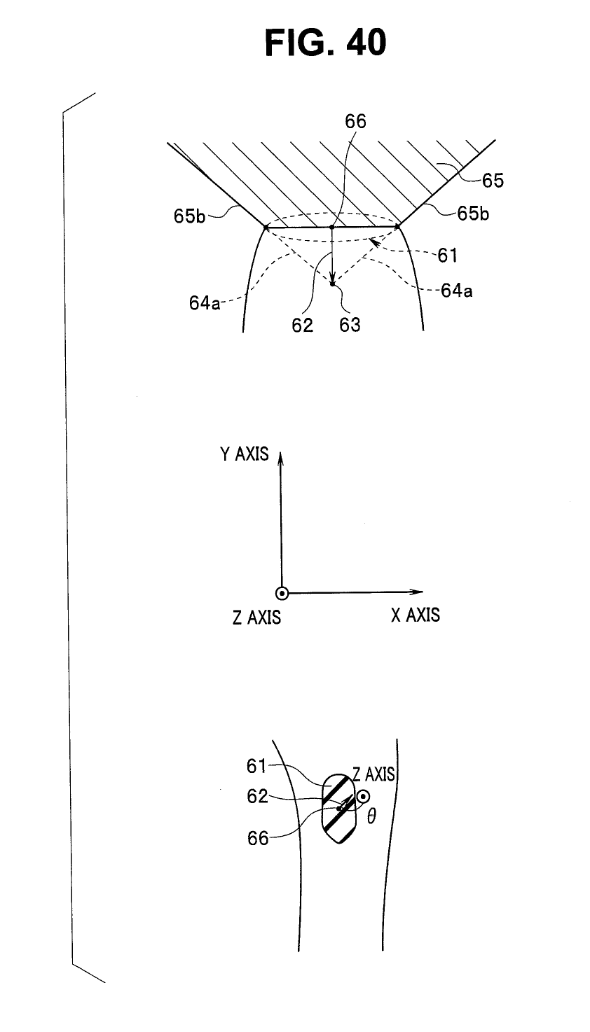

At a step following step S93, as illustrated in an uppermost diagram in FIG. 40, line segments (line segments illustrated with a dotted lines) 64a connecting the point light source 63 and two points facing to each other and sandwiching the barycenter 66 of the opening portion 61 are drawn, and a region (hatched region) of a polygon connecting line segments (line segments illustrated with solid lines) 65b extending from the two points toward the outside of the opening portion 61 and a line segment connecting the two points is colored in the color of the point light source and set as an index 65. In other words, the index 65 is formed by coloring, in the color of the point light source 63, a region outside the opening portion 61 within the angle between two line segments passing through the two points on the opening portion 61 facing to each other and sandwiching the barycenter 66 from the point light source 63.

Note that, when a Z axis is defined to be an axis orthogonal to a display screen and an angle θ between the normal 62 and the Z axis is equal to or smaller than a certain angle (for example, 45 degrees) as illustrated in a lowermost diagram in FIG. 40, the inside of the opening portion 61, which is hatched with bold lines, is colored and displayed.

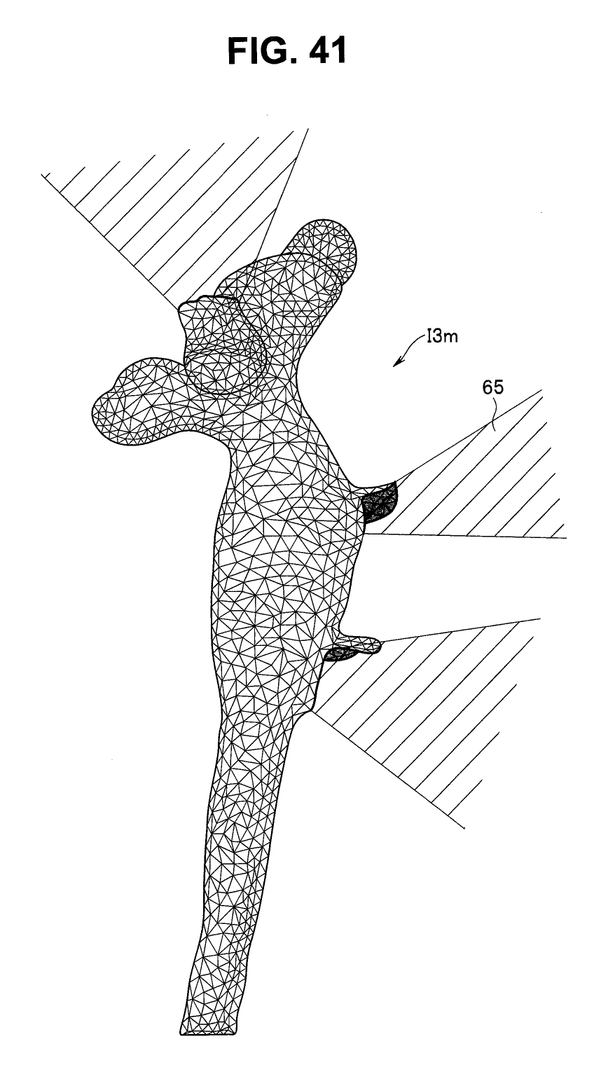

FIG. 41 illustrates a 3D model image I3m when the enhanced display and the index display are selected in the present modification.

As illustrated in FIG. 41, in addition to the enhanced display, the index (part hatched in FIG. 41) 65 as if light leaks from an opening adjacent to an unobserved region is displayed to indicate the unobserved region, and thus a situation in which an unobserved region equal to or larger than the defined area exists can be recognized in an easily visually recognizable state.

The following describes a ninth modification of the first embodiment. In the first embodiment and the modifications described above, a 3D model image viewed in a predetermined direction is generated and displayed as illustrated in, for example, FIGS. 13, 17, 20, and 23.

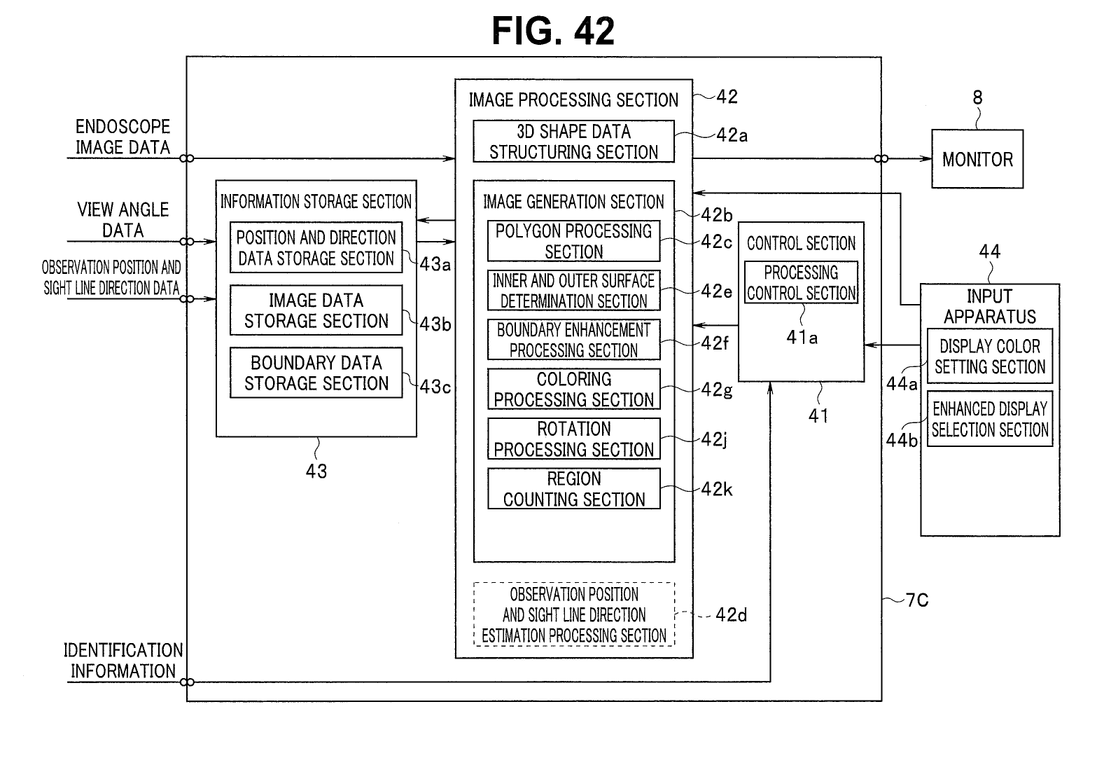

FIG. 42 illustrates the configuration of an image processing apparatus 7C in the present modification.

In the present modification, the image generation section 42b further includes a rotation processing section 42j configured to rotate a 3D model image, and a region counting section 42k configured to count the number of boundaries (regions), unobserved regions, or unstructured regions in addition to the configuration illustrated in FIG. 2 in the first embodiment.

The rotation processing section 42j rotates a 3D model image viewed in a predetermined direction around, for example, a core line so that, when the 3D model image viewed in a predetermined direction is a front image, the front image and a back image viewed from a back surface on a side opposite to the predetermined direction can be displayed side by side, and 3D model images viewed in a plurality of directions selected by the operator can be displayed side by side. In addition, overlooking of a boundary can be prevented.

For example, when the number of unstructured regions counted by the region counting section 42k is zero in a front image viewed in a predetermined direction, a 3D model image may be rotated by the rotation processing section 42j so that the number is equal to or larger than one (except for a case in which unstructured regions exist nowhere). When an unstructured region in three-dimensional model data cannot be visually recognized, the image generation section 42b may provide the three-dimensional model data with rotation processing, generate a three-dimensional model image in which the unstructured region is visually recognizable, and display the three-dimensional model image.

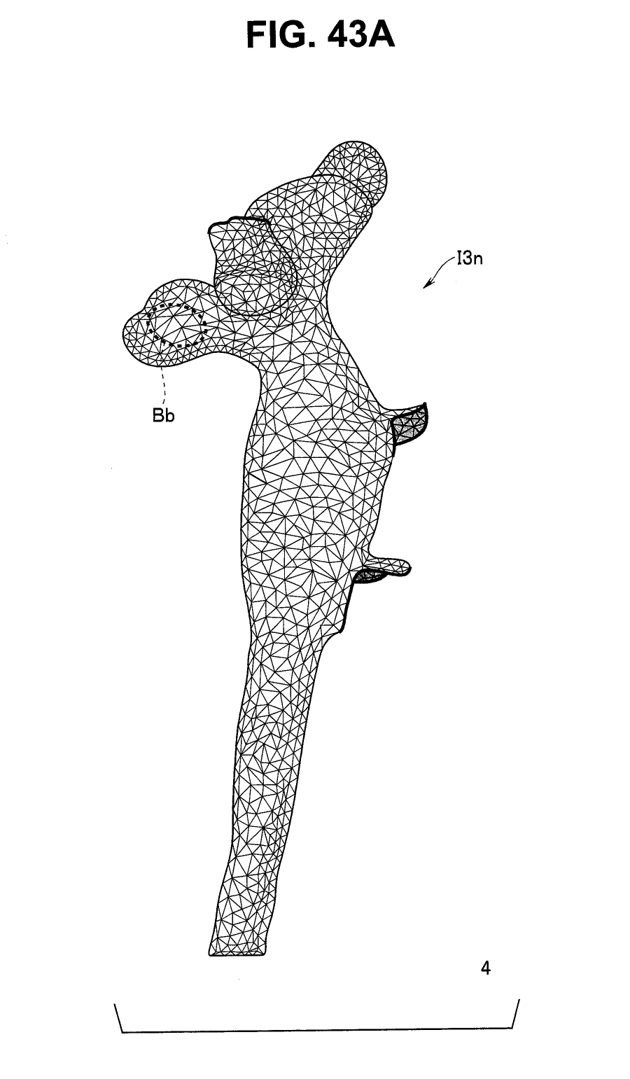

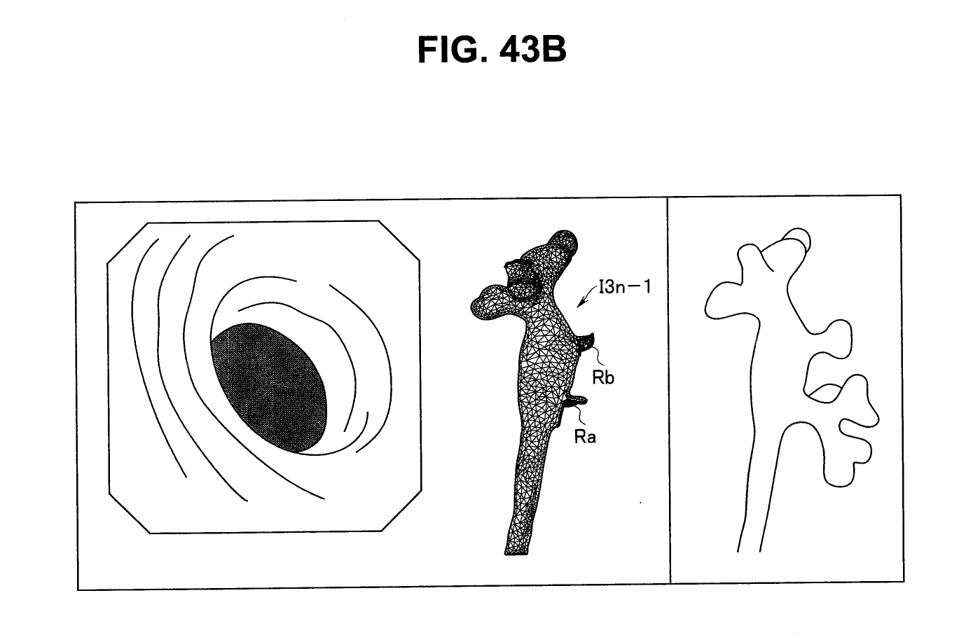

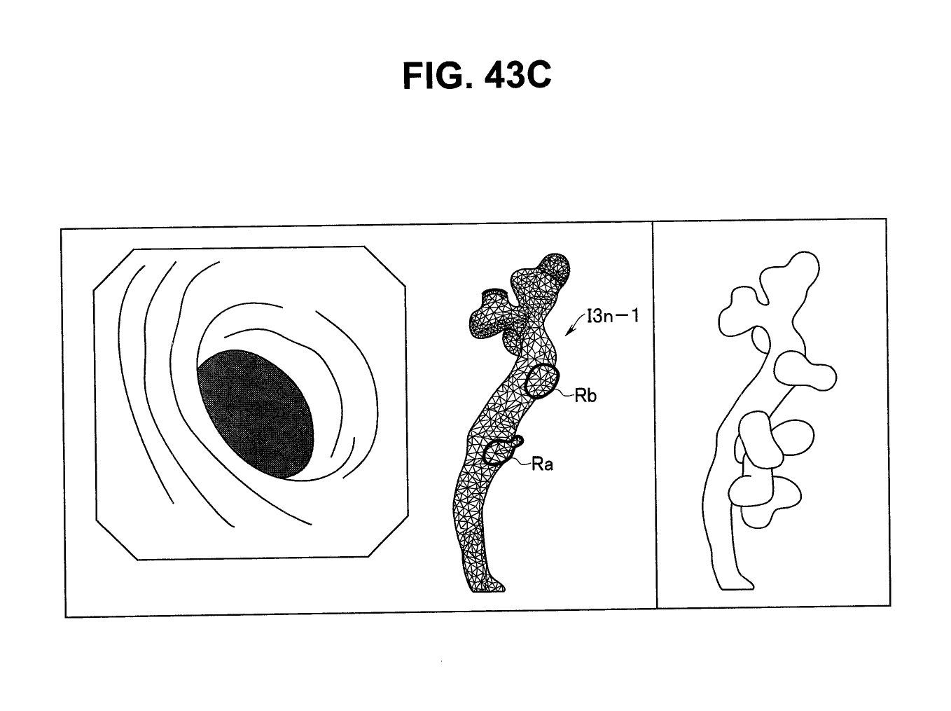

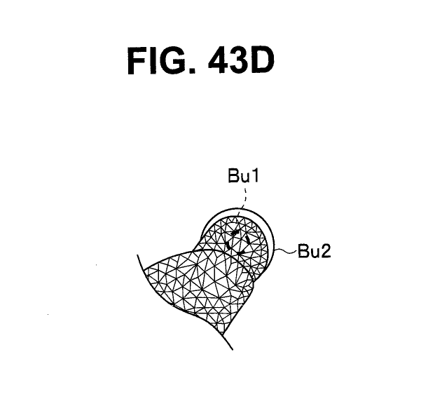

In place of, for example, the 3D model image I3d in which a boundary (or unobserved region) appearing on a front side when viewed in a predetermined direction is displayed in an enhanced manner, a back-side boundary Bb appearing when viewed from a back side may be illustrated with a dotted line in a color (for example, purple; note that a background color is light blue and thus distinguishable from purple) different from a color (for example, red) indicating a boundary appearing on the front side in a 3D model image I3n in the present modification as illustrated in FIG. 43A.

In the 3D model image I3o, a count value of discretely existing boundaries (regions) counted by the region counting section 42k may be displayed in the display screen of the monitor 8 (in FIG. 43A, the count value is four).

In the display illustrated in FIG. 43A, a boundary appearing on the back side, which does not appear when viewed in a predetermined direction (front), is displayed in a color different from a color illustrating a boundary on the front side to prevent overlooking of any boundary on the back side, and also the count value is displayed to effectively prevent overlooking of a boundary. In addition, effects same as the effects of the first embodiment are achieved.

Note that only a boundary or a boundary region may be displayed without displaying an observed 3D model shape. For example, only four boundaries (regions) in FIG. 43A may be displayed. In such a case, the boundaries (regions) are displayed floating in a space in an image. Alternatively, the outline of a 3D model shape may be displayed with, for example, a double-dotted and dashed line, and boundaries (regions) may be displayed on the outline of the 3D model shape, thereby displaying the positions and boundary shapes of the boundaries (regions) in the 3D shape in an easily recognizable manner Such display can effectively prevent boundary overlooking.

A 3D model image may be rotated and displayed as described below.

When it is sensed that an unstructured region is disposed and superimposed behind (on the back surface of) a structured region on the surface of the monitor 8 when viewed by the user and thus cannot be visually recognized by the user, the rotation processing section 42j may automatically rotate the 3D model image so that the unstructured region is disposed on the front side at which the unstructured region is easily visually recognizable.

When a plurality of unstructured regions exist, the rotation processing section 42j may automatically rotate the 3D model image so that an unstructured region having a large area is disposed on the front side.

For example, a 3D model image I3n-1 as a rotation processing target illustrated in FIG. 43B may be rotated and displayed so that an unstructured region having a large area is disposed on the front side as illustrated in FIG. 43C. Note that FIGS. 43B and 43C illustrate a state in which an endoscope image and the 3D model image I3n−1 are disposed on the right and left sides on the display screen of the monitor 8. The 3D shape of a renal pelvis and calyx modeled and displayed in the 3D model image I3n−1 is illustrated on the right side of the display screen.

When a plurality of unstructured regions exist, the rotation processing section 42j may automatically rotate a 3D model image so that an unstructured region nearest to the leading end position of the endoscope 2I is disposed on the front side.

Note that the unstructured region may be displayed in an enlarged manner. An unobserved region may be largely displayed in an enlarged manner to display the unstructured region in an easily visually recognizable manner.

For example, when an unstructured region Bu1 exists behind (on the back side) as illustrated with a dotted line in FIG. 43D, the unstructured region may be (partially) displayed in a visually recognizable manner by displaying an unstructured region Bu2 having an enlarged size larger than the size of a structured region part on the front side covering the unstructured region Bu1.

Note that not only an unstructured region behind (on the back side) but all unstructured regions may be displayed in an enlarged manner to display the unstructured region in a more easily visually recognizable manner.

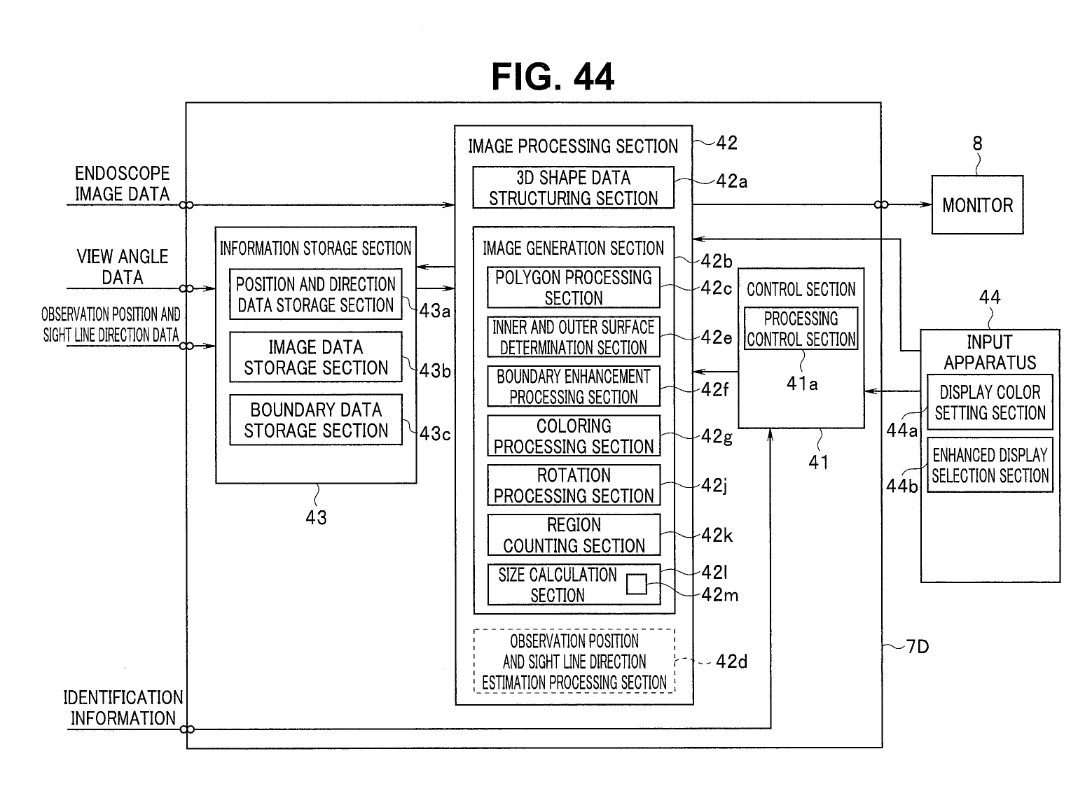

The following describes a tenth modification of the first embodiment. FIG. 44 illustrates an image processing apparatus 7D in the tenth modification. In the present modification, the image generation section 42b in the image processing apparatus 7C in the ninth modification illustrated in FIG. 42 further includes a size calculation section 42l configured to calculate the size of an unstructured region. The size calculation section 42l functions as a determination section 42m configured to determine whether the size of the unstructured region is equal to or smaller than a threshold. Note that the determination section 42m may be provided outside of the size calculation section 42l. The other configuration is same as the configuration in the ninth modification.

The size calculation section 42l in the present modification calculates the size of the area of each unstructured region counted by the region counting section 42k. Then, when the calculated size of the unstructured region is equal to or smaller than the threshold, processing of displaying (a boundary of) the unstructured region in an enhanced manner so that (the boundary of) the unstructured region is easily visually recognizable is not performed, and the unstructured region is not counted in the number of unstructured regions.

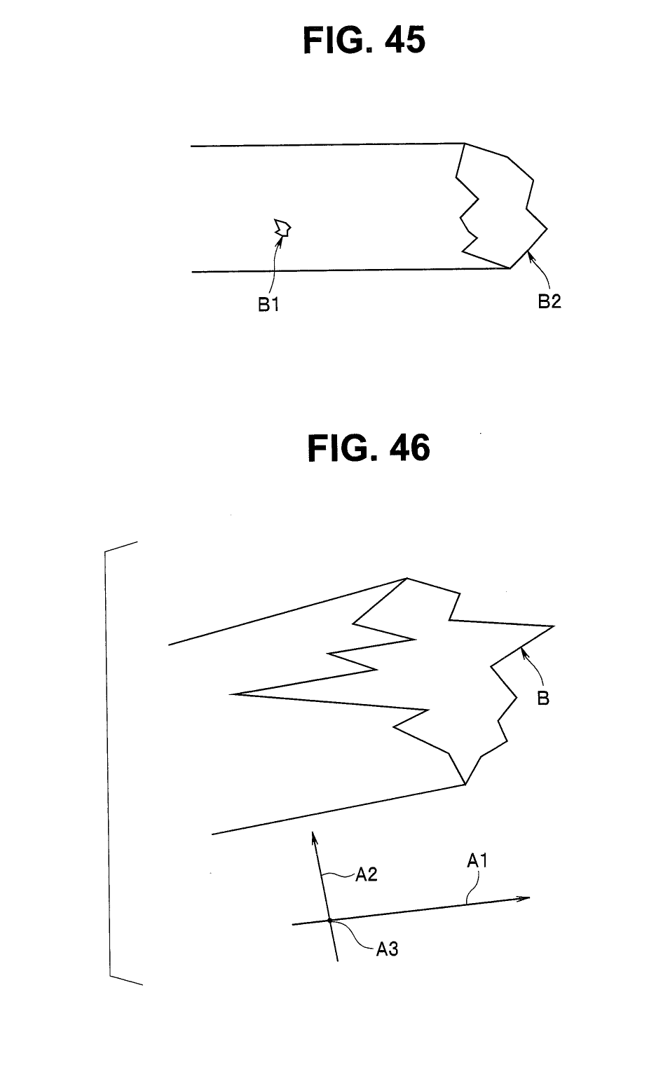

FIG. 45 illustrates 3D shape data including a boundary B1 having a size equal to or smaller than the threshold and a boundary B2 having a size exceeding the threshold. The boundary B2 is displayed in an enhanced manner in an easily visually recognizable color (for example, red) such as red, whereas the boundary B1 is a small area that does not need to be observed and thus is not provided with the enhancement processing or is provided with processing of blocking an opening at the boundary with polygons (or processing of blocking the opening with polygons to produce a pseudo observation region). In other words, an unstructured region including the boundary B1 having a size equal to or smaller than the threshold is not provided with processing of allowing visual recognition nor processing of facilitating visual recognition.

In the present modification, when the determination section 42m determines whether to perform the enhancement processing, the determination is not limited to a condition based on whether the area of an unstructured region or a boundary is equal to or smaller than the threshold as described above, but the determination may be made based on conditions described below.

That is, the determination section 42m does not perform the enhancement processing or generates a pseudo observed region when at least one of conditions A to C below is satisfied:

A. when the length of a boundary is equal to or smaller than a length threshold,
B. when the number of apexes included in the boundary is equal to or smaller than a threshold for the number of apexes, or
C. when in primary component analysis of the coordinates of the boundary, the difference between the maximum and minimum of a second primary component or the difference between the maximum and minimum of a third primary component is equal to or smaller than a component threshold.

FIG. 46 illustrates an explanatory diagram of the condition C. FIG. 46 illustrates 3D shape data of a lumen having a boundary B in a complicated shape at the right end, an axis A1 of a first primary component is aligned with a longitudinal direction of the lumen, an axis A2 of the second primary component is aligned with a direction orthogonal to the axis A1 of the first primary component in the sheet of FIG. 46, and an axis A3 of the third primary component is aligned with a direction orthogonal to the sheet.

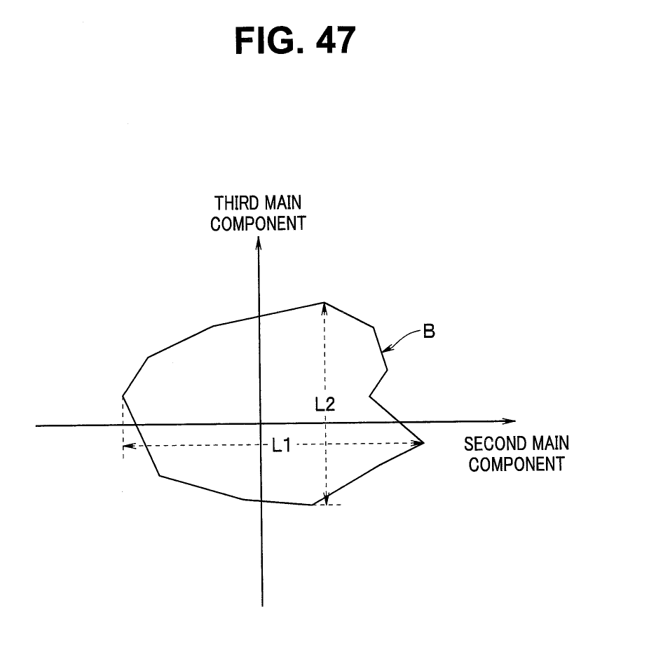

Subsequently, the coordinates of a boundary are projected onto a plane orthogonal to the axis A1 of the first primary component. FIG. 47 illustrates a diagram of the projection. The determination section 42m calculates lengths in directions parallel to respective axes of a plane illustrated in FIG. 47 and determines whether the difference between the maximum and minimum of the second primary component or the difference between the maximum and minimum of the third primary component is equal to or smaller than a component threshold. FIG. 47 illustrates a maximum length L1 of the second primary component and a maximum length L2 of the third primary component.

In the present modification, the effects of the ninth modification are achieved, and in addition, unnecessary display is not performed by not displaying a small boundary that does not need to be observed.

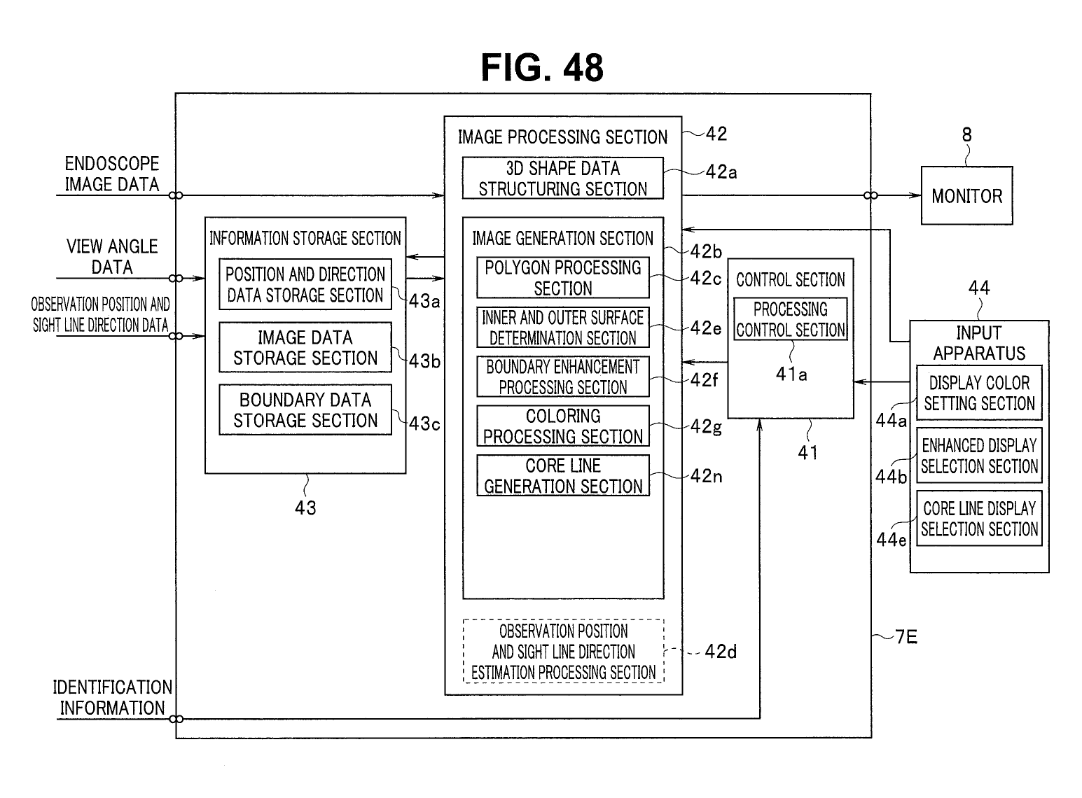

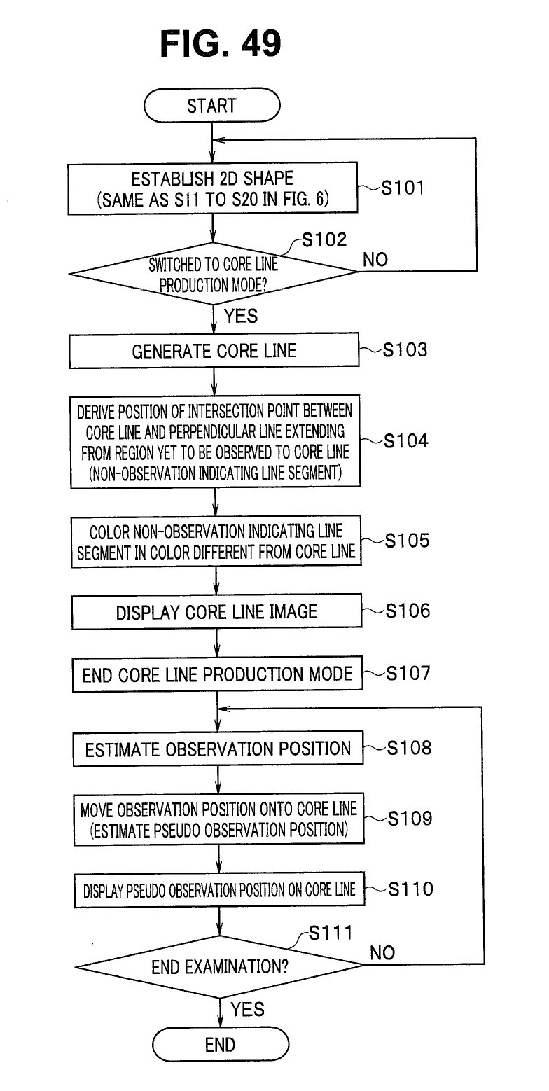

The following describes an eleventh modification of the first embodiment. FIG. 48 illustrates an image processing apparatus 7E in the eleventh modification. In addition to the image processing apparatus 7 illustrated in FIG. 2, the image processing apparatus 7E illustrated in FIG. 48 further includes a core line generation section 42n configured to generate a core line for 3D shape data. The input apparatus 44 includes a core line display selection section 44e configured to display a 3D model image with a core line.

In the present modification, processing same as processing in the first embodiment is performed when the input apparatus 44 does not perform selection for displaying a 3D model image with a core line by the core line display selection section 44e, or processing illustrated in FIG. 49 is performed when selection for display with a core line by the core line display selection section 44e is performed.

The following describes the processing illustrated in FIG. 49. When the processing illustrated in FIG. 49 is started, the image processing section 42 acquires a 2D image from the video processor 4 at step S101, and structures a 3D shape from 2D images inputted in a temporally substantially continuous manner. In a specific method, the 3D shape can be formed from the 2D images through processing same as steps S11 to S20 in FIG. 6 described above (by, for example, the method of marching cubes).

When it is determined that switching to a core line production mode is made at step S102, the 3D shape structuring is ended to transition to the core line production mode. The switching to the core line production mode is determined based on, for example, inputting through operation means by the operator or determination of the degree of progress of the 3D shape structuring by a processing apparatus.

After the switching to the core line production mode, a core line of the shape produced at step S101 is produced at step S103. Note that core line production processing can employ publicly known methods such as methods described in, for example, “Masahiro YASUE, Kensaku MORI, Toyofumi SAITO, et al., Thinning Algorithms for Three-Dimensional Gray Images and Their Application to Medical Images with Comparative Evaluation of Performance, Journal of The Institute of Electronics, Information and Communication Engineers, J79-D-H(10):1664-1674, 1996”, and “Toyofumi SAITO, Satoshi BANJO, Jun-ichiro TORIWAKI, An Improvement of Three Dimensional Thinning Method Using a Skeleton Based on the Euclidean Distance Transformation —A Method to Control Spurious Branches-, Journal of The Institute of Electronics, Information and Communication Engineers, (J84-D2:1628-1635) 2001”.

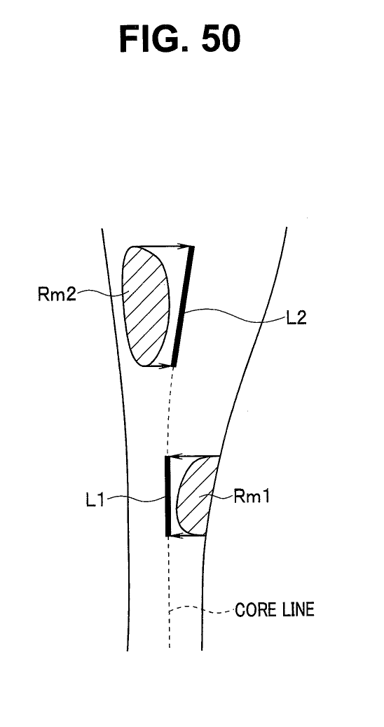

After the core line is produced, the position of an intersection point between the core line and a perpendicular line extending toward the core line from a colored region in a different color illustrating an unobserved region in the 3D shape is derived at step S104. The derivation is simulated in FIG. 50. In FIG. 50, Rm1 and Rm2 (colored regions hatched in FIG. 50) illustrating unobserved regions exist on the 3D shape. Perpendicular lines extend from the unobserved regions Rm1 and Rm2 toward the core line already formed at step S103 and illustrated with a dotted line. Intersection points between the perpendicular lines and the core line are indicated by line segments L1 and L2 on the core line illustrated with solid lines. Then, at step S105, the line segments L1 and L2 are each colored in a color (for example, red) different from the color of the other region of the core line.

Through the processing performed so far, the core line illustrating an observed region and an unobserved region in a pseudo manner is displayed (step S106).

After the formation and the display of the core line, the core line production mode is ended (step S107).

Subsequently, at step S108, the observation position and sight line direction estimation processing section estimates an observation position and a sight line direction of the endoscope based on acquired observation position and sight line direction data.

In addition, calculation on movement of the observation position onto the core line is performed at step S109 to illustrate the observation position estimated at step S108 on the core line in a pseudo manner. At step S109, the estimated observation position is moved to a point on the core line at which the distance between the estimated observation position and the core line is minimized.

At step S110, the pseudo observation position estimated at step S109 is displayed together with the core line. Accordingly, the operator can determine whether an unobserved region is approached.

The display is repeated from step S108 until determination to end an examination is made (step S111).

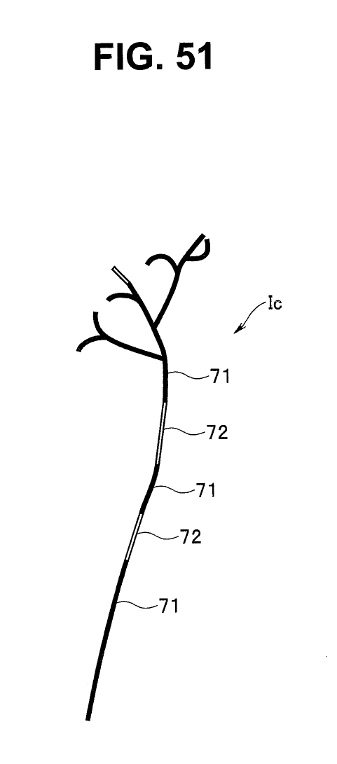

FIG. 51 illustrates an exemplary state when step S106 is ended, and illustrates a core line image Ic generated in the observation region including the unobserved regions Rm1 and Rm2 In FIG. 51, a core line 71 and a core line including a line segment 72 are displayed in colors different from each other, and the user such as an operator can easily visually recognize that an unobserved region exists based on the core line including the line segment 72.

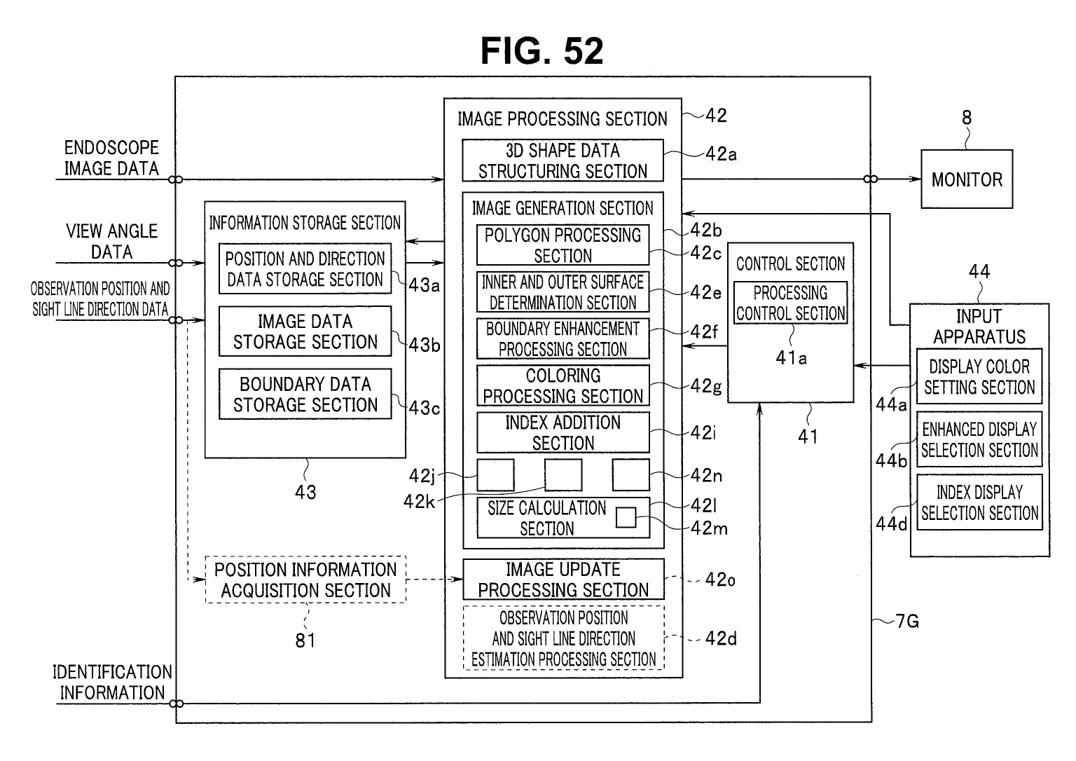

An image processing apparatus having the functions in the first embodiment to the eleventh modification described above may be provided. FIG. 52 illustrates an image processing apparatus 7G in a twelfth modification having such functions. The respective components of the image generation section 42b and the input apparatus 44 in the image processing apparatus 7G illustrated in FIG. 52 are already described, and thus any duplicate description will be omitted. According to the present modification, the user such as an operator has an increased number of options for selecting the display format of a 3D model image when displayed on the monitor 8, and in addition to the above-described effects, a 3D model image that satisfies a wider range of requirement by the user can be displayed.

Note that, in the first embodiment including the above-described modifications, the endoscope 2A and the like are not limited to a flexible endoscope including the flexible insertion section 11 but are also applicable to a rigid endoscope including a rigid insertion section.

The present invention is applicable to, in addition to a case of a medical endoscope used in the medical field, a case in which the inside of, for example, a plant is observed and examined by using an industrial endoscope used in the industrial field.

Parts of the embodiment including the above-described modifications may be combined to achieve a different embodiment. In addition, only the enhanced display may be performed without coloring the inner surface (inner wall surface or inner wall region) and the outer surface (outer wall surface or outer wall region) of a polygon in different colors.

A plurality of claims may be integrated into one claim, and the contents of one claim may be divided into a plurality of claims.

What is claimed is:

1. An image processing apparatus comprising:
a three-dimensional model structuring section configured to generate, when an image pickup signal related to a region in a subject is inputted from an image pickup apparatus configured to pick up an image of an inside of the subject, three-dimensional data representing a shape of the region based on the image pickup signal; and
an image generation section configured to perform, on the three-dimensional data generated by the three-dimensional model structuring section, processing of allowing visual recognition of a boundary region between a structured region that is a region, an image of which is picked up by the image pickup apparatus, and an unstructured region that is a region, an image of which is yet to be picked up by the image pickup apparatus, and generate a three-dimensional image.

2. The image processing apparatus according to claim 1, wherein
when a first image pickup signal related to a first region in the subject is inputted from the image pickup apparatus, the three-dimensional model structuring section generates three-dimensional data representing a shape of the first region based on the first image pickup signal,
the image generation section generates a three-dimensional image based on the three-dimensional data representing the shape of the first region and outputs the three-dimensional image to a display section,
when a second image pickup signal related to a second region including a region different from the first region is inputted from the image pickup apparatus after the first image pickup signal is inputted, the three-dimensional model structuring section generates three-dimensional data representing a shape of the second region based on the second image pickup signal, and
the image generation section generates three-dimensional images of the first region and the second region based on the three-dimensional data representing the shape of the second region and outputs the three-dimensional images to the display section.

3. The image processing apparatus according to claim 2, wherein the three-dimensional model structuring section sets the second image pickup signal to be, among image pickup signals inputted from the image pickup apparatus after the first image pickup signal, an image pickup signal in which a predetermined change amount is detected for the first region included in the first image pickup signal.

4. The image processing apparatus according to claim 2, wherein the image generation section generates a three-dimensional image by synthesizing the three-dimensional data representing the shape of the first region and the three-dimensional data representing the shape of the second region and outputs the three-dimensional image to the display section.

5. The image processing apparatus according to claim 2, wherein
the three-dimensional model structuring section stores three-dimensional data generated based on the first image pickup signal and representing the shape of the first region in a storage section and additionally stores three-dimensional data generated based on the second image pickup signal and representing the shape of the second region in the storage section, and
the image generation section generates a three-dimensional image by synthesizing the three-dimensional data representing the shape of the first region and the three-dimensional data representing the shape of the second region that are stored in the storage section, and outputs the three-dimensional image to the display section.

6. The image processing apparatus according to claim 2, wherein
the three-dimensional model structuring section stores the first image pickup signal in a storage section instead of generating the three-dimensional data representing the shape of the first region when the first image pickup signal is inputted, and stores the second image pickup signal in the storage section instead of generating the three-dimensional data representing the shape of the second region when the second image pickup signal is inputted, and
the image generation section generates a three-dimensional image based on the first image pickup signal and the second image pickup signal stored in the storage section and outputs the three-dimensional image to the display section.

7. The image processing apparatus according to claim 1, further comprising a position information acquisition section configured to acquire leading end position information that is information indicating a leading end position of an insertion section that is inserted into the subject, wherein the three-dimensional model structuring section and the image generation section generates a three-dimensional image based on change of the leading end position information along with an operation of inserting the insertion section.

8. The image processing apparatus according to claim 1, wherein, when generating a three-dimensional image of the subject, the image generation section performs processing of differentiating a color of an inner wall region and a color of an outer wall region in three-dimensional data structured by the three-dimensional model structuring section.

9. The image processing apparatus according to claim 1, wherein the image generation section performs, on three-dimensional data structured by the three-dimensional model structuring section, processing of smoothing the boundary region of a lumen between an unstructured region and a structured region in a three-dimensional image of the subject and expressing the boundary region in a substantially curved line.

10. The image processing apparatus according to claim 1, wherein, when generating a three-dimensional image of the subject, the image generation section adds index information for a surrounding region of the unstructured region.

11. The image processing apparatus according to claim 1, wherein, when the unstructured region cannot be visually recognized, the image generation section performs processing of allowing visual recognition of the unstructured region that cannot be visually recognized by performing rotation processing on three-dimensional data structured by the three-dimensional model structuring section.

12. The image processing apparatus according to claim 1, wherein, when the unstructured region cannot be visually recognized, the image generation section performs processing of illustrating the unstructured region that cannot be visually recognized in a color different from a color of any other unstructured region.

13. The image processing apparatus according to claim 1, wherein the image generation section performs processing of calculating a number of the unstructured region in three-dimensional data structured by the three-dimensional model structuring section, and displaying the number of the unstructured region on a display section.

14. The image processing apparatus according to claim 1, wherein the image generation section includes:
a size calculation section configured to calculate a size of each of the unstructured region in three-dimensional data structured by the three-dimensional model structuring section; and
a determination section configured to determine whether the size calculated by the size calculation section is smaller than a predetermined threshold, and
the image generation section does not perform processing of allowing visual recognition on the unstructured region, the size of which is determined to be smaller than the predetermined threshold by the determination section.

15. The image processing apparatus according to claim 1, wherein the image generation section performs, on three-dimensional data structured by the three-dimensional model structuring section, processing of allowing visual recognition of only the boundary region of a lumen between an unstructured region and a structured region in a three-dimensional image of the subject.

16. The image processing apparatus according to claim 1, wherein
the image generation section further includes a core line generation section configured to generate core line data of three-dimensional data structured by the three-dimensional model structuring section, and
generates, for the core line data, a core line image in which a region corresponding to the unstructured region has a different color.

17. The image processing apparatus according to claim 1, wherein the image generation section performs, on three-dimensional data structured by the three-dimensional model structuring section, processing of setting a color of the boundary region of a lumen between an unstructured region and a structured region in a three-dimensional image of the subject to be variable.

18. The image processing apparatus according to claim 1, wherein the unstructured region is a region in the subject that is yet to be observed with an endoscope.

19. An image processing method comprising:
generating, by a three-dimensional model structuring section, when an image pickup signal related to a region in a subject is inputted from an image pickup apparatus configured to pick up an image of an inside of the subject, three-dimensional data representing a shape of the region based on the image pickup signal; and
performing, by an image generation section, on the three-dimensional data generated by the three-dimensional model structuring section, processing of allowing visual recognition of a boundary region between a structured region that is a region, an image of which is picked up by the image pickup apparatus, and an unstructured region that is a region, an image of which is yet to be picked up by the image pickup apparatus, and generating a three-dimensional image.
